(19) United States
(12) Patent Application Publication
(10) Pub. No.: US 2025/0177393 A1
(43) Pub. Date: Jun. 5, 2025

(54) MODULATORS OF MYC FAMILY PROTO-ONCOGENE PROTEIN

(71) Applicant: Nalo Therapeutics, Palo Alto, CA (US)

(72) Inventors: William Greenlee, Teaneck, NJ (US); Stephen J. Shuttleworth, Buckinghamshire (GB); Keith Wilson, Belmont, CA (US)

(21) Appl. No.: 18/840,424

(22) PCT Filed: Feb. 24, 2023
(86) PCT No.: PCT/US2023/063211

§ 371 Date: Aug. 21, 2024

Related U.S. Application Data
(60) Provisional application No. 63/313,863, filed on Feb. 25, 2022.

(51) Int. Cl. A61K 31/506; C07D 401/14; C07D 403/14; C07D 405/14; C07D 409/14; C07D 471/10; C07D 491/10; C07F 9/6584

(52) U.S. Cl. CPC A61K 31/506; C07D 401/14; C07D 403/14; C07D 405/14; C07D 409/14; C07D 471/10; C07D 491/10; C07F 9/6584

(57) ABSTRACT

Disclosed herein are compounds and compositions having potency in the modulation of Myc family proteins. Such compounds and compositions can be used in the treatment of proliferative diseases, such as cancer, or in the treatment of disease where modulation of Myc family proteins is desired. Also disclosed herein are methods of using said compounds and compositions.

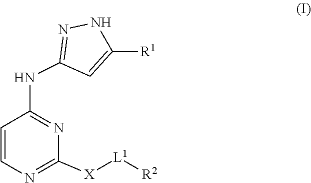

### CROSS REFERENCE TO RELATED APPLICATIONS

This application claims priority to and the benefit of U.S. Provisional Patent Application No. 63/313,863 filed Feb. 25, 2022, which is incorporated herein by reference in its entirety.

### BACKGROUND

The MYC proto-oncogene family comprises three members: C-MYC, MYCN, and MYCL. These oncogenes encode c-Myc, N-Myc, and L-Myc oncoproteins, respectively, which belong to a family of “super-transcription factors” that regulate the transcription of more than 15% of the entire genome. Recent studies in mouse models have suggested that the regulation of oncogenic Myc proteins could potentially lead to the development of cancer therapeutics, as it has been demonstrated that even transient inactivation of Myc causes tumor regression. However, the development of drugs and therapeutics that directly targets Myc proteins has met with two major challenges. First, Myc proteins lack a well-defined active site for the binding of small molecules, thus providing challenges for the functional modulation or inhibition of their activities. Second, Myc proteins are predominantly located in cell nuclei, and targeting nuclear Myc proteins with antibodies can be technically challenging. These challenges have spawned strategies for indirect regulation of Myc proteins.

For example, amplification and overexpression of N-Myc can lead to tumorigenesis. Excess N-Myc is associated with a variety of tumors, e.g., neuroblastomas. MYCN can also be activated in tumors through somatic mutation.

C-Myc can also be constitutively expressed in various cancers such as cervix, colon, breast, lung and stomach cancers. Such constitutive expression can lead to increased expression of other genes that are involved in cell proliferation.

Amplification of the, e.g., N-Myc gene in patients frequently results in poor health outcomes. However, strategies for direct modulation of Myc proteins remain elusive, as the Myc proteins are not easily targeted.

Therefore, an ongoing need exists for small-molecule therapeutic modulators of Myc proteins for the treatment of various ailments, diseases and disorders, e.g., cancer.

### SUMMARY

The present disclosure provides compounds and compositions that are useful as Myc protein modulators, and methods of using the same. Furthermore, the present disclosure contemplates using disclosed compounds and compositions as direct modulators of Myc proteins in the treatment of proliferative disease, such as cancer, or in the treatment of diseases where modulation of Myc family proteins is desired.

For example, the present disclosure provides a compound of Formula I:

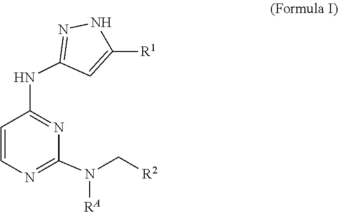

or a pharmaceutically acceptable salt, stereoisomer, and/or N-oxide thereof, wherein:
R¹ is C₃-C₆ cycloalkyl;
R² is 5-10 membered heterocyclyl having at least one nitrogen, wherein the heterocyclyl is optionally substituted by one or two halo, oxo, hydroxyl, or C₁-C₄ alkyl; and
R^{A} is selected from H and C₁-C₆ alkyl;
wherein when R² is a 6-membered monocyclic heterocyclyl, R¹ is cyclopentyl.

Pharmaceutical compositions comprising a disclosed compound or a pharmaceutically acceptable salt, stereoisomer, and/or N-oxide thereof, as described herein, for example a disclosed pharmaceutical composition may include least one or more pharmaceutically acceptable carriers, diluents, stabilizers, excipients, dispersing agents, suspending agents, and/or thickening agents. The present disclosure also provides a method of manufacturing of the compounds described herein, or a pharmaceutically acceptable salt, stereoisomer, and/or N-oxide thereof.

A method of modulating the amount and activity of a Myc family protein (i.e., C-Myc, N-Myc, L-Myc, or human Myc) is also provided, for example, an activity of a Myc family protein may be modulated in a cell by contacting a cell with an effective amount of a compound as described herein, or a pharmaceutically acceptable salt, stereoisomer, and/or N-oxide thereof.

The present disclosure also provides a method of treating a Myc family protein associated disease in a subject in need thereof, the method comprising administering a therapeutically effective amount of a compound described herein, or a pharmaceutically acceptable salt, stereoisomer, and/or N-oxide thereof, including embodiments in any examples, tables, or figures. In some embodiments, the subject is a human subject and the disease is a proliferative disease, such as cancer.

### DETAILED DESCRIPTION

### Definitions

Unless defined otherwise, all technical and scientific terms used herein have the same meaning as commonly understood by one of ordinary skill in the art to which this disclosure pertains.

It is understood that the definitions provided herein are not intended to be mutually exclusive. Accordingly, some chemical moieties may fall within the definition of more than one term.

The term “alkoxy” as used herein refers to a straight or branched alkyl group attached to oxygen (alkyl-O—). Exemplary alkoxy groups include, but are not limited to, alkoxy groups of 1-6 or 2-6 carbon atoms, referred to herein as C₁₋₆alkoxy, and C₂₋₆alkoxy, respectively. Exemplary alkoxy groups include, but are not limited to methoxy, ethoxy, isopropoxy, n-butoxy, tert-butoxy, sec-butoxy, n-pentoxy, n-hexoxy, 1,2-dimethylbutoxy, etc.

The term “alkyl” as used herein refers to a saturated straight or branched hydrocarbon. Exemplary alkyl groups include, but are not limited to, straight or branched hydrocarbons of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

As used herein, the term “alkylene” refers to a di-radical alkyl group. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

The term “alkenyl” as used herein refers to an unsaturated straight or branched hydrocarbon having at least one carbon-carbon double bond. Exemplary alkenyl groups include, but are not limited to, a straight or branched group of 2-6 or 3-4 carbon atoms, referred to herein as C₂₋₆alkenyl, and C₃₋₄alkenyl, respectively. Exemplary alkenyl groups include, but are not limited to, vinyl, allyl, butenyl, pentenyl, etc.

The term “alkynyl” as used herein refers to an unsaturated straight or branched hydrocarbon having at least one carbon-carbon triple bond. Exemplary alkynyl groups include, but are not limited to, straight or branched groups of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

As used herein, the terms “alkenylene,” “alkynylene,” “arylene,” “arylalkylene,” and “alkylarylene” refer to di-radical alkenyl, alkynyl, aryl, arylalkyl, and alkylaryl groups, respectively.

As used herein, the term “azido” refers to group —N₃.

As used herein, the term “carboxyl,” “carboxy” or “carboxylate” refers to —CO₂H or salts thereof.

As used herein, the term “carbamoyl” refers to the group NH₂CO—.

The terms “cycloalkyl” or a “carbocyclic group” as used herein refers to a saturated or partially unsaturated hydrocarbon group of, for example, 3-10, 3-6, or 4-6 carbons, referred to herein as C₃₋₁₀cycloalkyl, or C₄₋₆cycloalkyl, respectively, and which may be monocyclic or bicyclic ring structures, e.g. 4-9 or 4-6 membered saturated ring structures, including bridged, fused or spirocyclic rings. Exemplary cycloalkyl groups include, but are not limited to, adamantanyl, cyclohexyl, cyclopentyl, cyclopentenyl, cyclobutyl, cyclopropyl, and indanyl.

As used herein, the groups

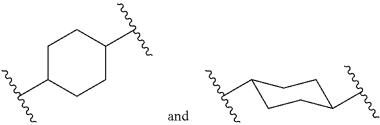 [SMILES: *C1CCC(*)CC1 ; *C1CCC(*)CC1]

and are used interchangeably and refer to a cyclohexyl group.

As used herein, the term “cyano” and “carbonitrile” refer to the group —CN.

As used herein, the term “formyl” refers to the group —C(O)H.

As used herein, the term “guanidino” refers to the group —NHC(═NH)NH₂.

As used herein, the terms “halo” and “halogen” are used in the conventional sense to refer to a chloro, bromo, fluoro or iodo substituent.

As used herein, the terms “hydroxy” and “hydroxyl” refer to the group —OH.

The terms “heteroaryl” or “heteroaromatic group” as used herein refers to a monocyclic aromatic 5-6 membered ring system containing one or more heteroatoms, for example one to three heteroatoms, such as nitrogen, oxygen, and sulfur. Where possible, said heteroaryl ring may be linked to the adjacent radical though carbon or nitrogen. Examples of heteroaryl rings include but are not limited to furan, thiophene, pyrrole, thiazole, oxazole, isothiazole, isoxazole, imidazole, pyrazole, triazole, pyridine or pyrimidine etc.

The terms “heterocyclyl” or “heterocyclic group” are art-recognized and refer to e.g. saturated or partially unsaturated, 4-10 membered monocyclic or bicyclic ring structures, or e.g. 4-9 or 4-6 membered saturated ring structures, including bridged, fused or spirocyclic rings, and whose ring structures include one to three heteroatoms, such as nitrogen, oxygen, and sulfur. Where possible, heterocyclyl rings may be linked to the adjacent radical through carbon or nitrogen. Examples of heterocyclyl groups include, but are not limited to, pyrrolidine, piperidine, morpholine, thiomorpholine, piperazine, oxetane, azetidine, tetrahydrofuran or dihydrofuran etc.

As used herein, the term “nitro” refers to the group —NO₂.

As used herein, the term “oxo” refers to the group (═O) or (O).

As used herein, the term “isomers” refers to compounds comprising the same numbers and types of atoms or components, but with different structural arrangement and connectivity of the atoms.

As used herein, the term “tautomer” refers to one of two or more structural isomers which readily convert from one isomeric form to another and which exist in equilibrium.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. Substituents around a carbocyclic or heterocyclic ring may be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the present disclosure embrace both solvated and unsolvated forms. In one embodiment, a disclosed compound is amorphous. In one embodiment, a disclosed compound is a single polymorph. In another embodiment, a disclosed compound is a mixture of polymorphs. In another embodiment, a disclosed compound is in a crystalline form.

The present disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those disclosed in the examples herein by substituting an isotopically labeled reagent for a non-isotopically labeled reagent

As used herein, singular articles such as “a,” “an” and “the” and similar referents in the context of describing the elements are to be construed to cover both the singular and the plural, unless otherwise indicated herein or clearly contradicted by context. Recitation of ranges of values herein are merely intended to serve as a shorthand method of referring individually to each separate value falling within the range, including the upper and lower bounds of the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (i.e., “such as”) provided herein, is intended merely to better illuminate the embodiments and does not pose a limitation on the scope of the claims unless otherwise stated.

In some embodiments, where the use of the term “about” is before a quantitative value, the present disclosure also includes the specific quantitative value itself, unless specifically stated otherwise. As used herein, the term “about” refers to a ±10% variation from the nominal value unless otherwise indicated or inferred. Where a percentage is provided with respect to an amount of a component or material in a composition, the percentage should be understood to be a percentage based on weight, unless otherwise stated or understood from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present disclosure remains operable. Moreover, two or more steps or actions can be conducted simultaneously.

As used herein, a dash (“-”) that is not between two letters or symbols refers to a point of bonding or attachment for a substituent. For example, —NH₂ is attached through the nitrogen atom.

As used herein, the terms “active agent,” “drug,” “pharmacologically active agent” and “active pharmaceutical ingredient” are used interchangeably to refer to a compound or composition which, when administered to a subject, induces a desired pharmacologic or physiologic effect by local or systemic action or both.

As used herein, the term “prodrug” refers to compounds that are transformed in vivo to provide a compound or pharmaceutically acceptable salt, hydrate or solvate of the compound described herein. The transformation can occur by various mechanisms (i.e., esterase, amidase, phosphatase, oxidative and/or reductive metabolism) in various locations (i.e., in the intestinal lumen or upon transit into the intestine, blood, or liver).

As used herein, the term “modulator” refers to a compound or composition that increases or decreases the level of a target or the function of a target, which may be, but is not limited to, a Myc family protein, such as c-Myc, N-Myc, L-Myc and human Myc.

As used herein, the term “degrader” refers to a compound or composition that decreases the amount of a target or the activity of a target. In some embodiments, the target may be, but is not limited to, a Myc family protein comprising c-Myc, N-Myc, L-Myc and human Myc.

As used herein, the term “degrading” refers to a method or process that decreases the amount of a target or the activity of a target. In some embodiments, the target may be, but is not limited to, a Myc family protein comprising c-Myc, N-Myc, L-Myc and human Myc.

As used herein, the term “Myc family protein” refers to any one of the proteins c-Myc, N-Myc, or L-Myc as described herein. In some embodiments, a Myc protein is a c-Myc protein. In some embodiments, a Myc protein is a N-Myc protein. In some embodiments, a Myc protein is a L-Myc protein. In some embodiments, a Myc protein is a human c-Myc protein. In some embodiments, a Myc protein is a human N-Myc protein. In some embodiments, a Myc protein is a human L-Myc protein. In some embodiments, a Myc family protein is a human Myc family protein.

As used herein, the terms “N-Myc” and “MycN” can be used interchangeably and refer to the protein “V-Myc myelocytomatosis viral related oncogene, neuroblastoma derived” and include the wildtype and mutant forms of the protein. In some embodiments, MycN refers to the protein associated with one or more of database entries of Entrez Gene 4613, OMIM 164840, UniProt P04198, and RegSeq NP_005369.

As used herein, the term “c-Myc” refers to the protein “V-Myc myelocytomatosis viral oncogene” and include the wildtype and mutant forms of the protein. In some embodiments, c-Myc refers to the protein associated with one or more of database entries of Entrez Gene 4609, OMIM 190080, UniProt P01106, and RegSeq NP_002458.

As used herein, the term “L-Myc” refers to the protein “V-Myc myelocytomatosis viral oncogene homolog, lung carcinoma derived” and include the wildtype and mutant forms of the protein. In some embodiments, L-Myc refers to the protein associated with one or more of database entries of Entrez Gene 4610, OMIM 164850, UniProt P12524, and RegSeq NP_001028253.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines, felines, equines, bovines, ovines, rodentia, etc. and primates, i.e., non-human primates, and humans. Non-human animal models, i.e., mammals, non-human primates, murines, lagomorpha, etc. may be used for experimental investigations.

As used herein, the terms “treating,” “treatment,” and the like, refer to obtaining a desired pharmacologic and/or physiologic effect, such as reduction of tumor burden. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human and includes: (a) preventing the disease or a symptom of a disease from occurring in a subject which may be predisposed to the disease but has not yet been diagnosed as having it (i.e., including diseases that may be associated with or caused by a primary disease); (b) inhibiting the disease, i.e., arresting its development; and (c) relieving the disease, i.e., causing regression of the disease (i.e., reduction in of tumor burden). In some embodiments, certain methods described herein treat cancer associated with the signaling pathway of a Myc family protein, such as c-Myc, N-Myc, L-Myc or human Myc.

As used herein, the term “pharmaceutically acceptable salt” refers to a salt which is acceptable for administration to a subject. It is understood that such salts, with counter ions, will have acceptable mammalian safety for a given dosage regime. Such salts can also be derived from pharmaceutically acceptable inorganic or organic bases and from pharmaceutically acceptable inorganic or organic acids, and may comprise organic and inorganic counter ions. The neutral forms of the compounds described herein may be converted to the corresponding salt forms by contacting the compound with a base or acid and isolating the resulting salts.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like.

Other examples of salts include anions of the compounds of the present disclosure compounded with a suitable cation such as N⁺, NH₄⁺, and NW₄⁺ (where W can be a C₁-C₈alkyl group), and the like. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Compounds included in the present compositions that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

As used herein, the terms “determining,” “measuring,” “assessing,” and “assaying” are used interchangeably and include both quantitative and qualitative determinations.

As used herein, the phrase “signaling pathway” refers to a series of interactions between cellular components, both intracellular and extracellular, that conveys a change to one or more other components in a living organism, which may cause a subsequent change to additional component. Optionally, the changes conveyed by one signaling pathway may propagate to other signaling pathway components. Examples of cellular components include, but are not limited to, proteins, nucleic acids, peptides, lipids and small molecules.

As used herein, the terms “effective amount” and “therapeutically effective amount” are used interchangeably and refer to the amount of a compound that, when administered to a mammal or other subject for treating a disease, condition, or disorder, is sufficient to affect such treatment for the disease, condition, or disorder. The “effective amount” or “therapeutically effective amount” will vary depending on the compound, the disease and its severity and the age, weight, etc., of the subject to be treated.

As used herein, the terms “pharmaceutically acceptable excipient,” “pharmaceutically acceptable diluent,” “pharmaceutically acceptable carrier,” and “pharmaceutically acceptable adjuvant” refer to an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. The phrase “a pharmaceutically acceptable excipient, diluent, carrier and adjuvant” as used in the specification and claims includes both one and more than one such excipient, diluent, carrier, and adjuvant.

As used herein, the term “pharmaceutical composition” is meant to encompass a composition suitable for administration to a subject, such as a mammal, especially a human. In general a “pharmaceutical composition” is sterile, and free of contaminants that are capable of eliciting an undesirable response within the subject (i.e., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

Generally, reference to or depiction of a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P and ³⁵S are thus within the scope of the present technology. Procedures for inserting such labels into the compounds of the present technology will be readily apparent to those skilled in the art based on the disclosure herein.

Unless the specific stereochemistry is expressly indicated, all chiral, diastereomeric, and racemic forms of a compound are intended. Thus, compounds described herein include enriched or resolved optical isomers at any or all asymmetric atoms as are apparent from the depictions. Racemic mixtures of (R)-enantiomer and (S)-enantiomer, and enantio-enriched stereoisomeric mixtures comprising of (R)- and (S)-enantiomers, as well as the individual optical isomers can be isolated or synthesized so as to be substantially free of their enantiomeric or diastereomeric partners, and these stereoisomers are all within the scope of the present technology.

The compounds described herein may exist as solvates, especially hydrates, and unless otherwise specified, all such solvates and hydrates are intended. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates, among others. The identification and preparation of any particular solvate is within the skill of the ordinary artisan of synthetic organic or medicinal chemistry.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### Compounds

The disclosure is generally directed to compounds that modulate (e.g., degrade) MycN and/or MycC, and may therefore have significant antineoplastic properties. The disclosed compounds and pharmaceutical compositions thereof find use in a variety of applications in which the modulation of the amount and activity of a Myc protein is desired, including use as potent antineoplastic agents.

Thus provided herein, in part, is a compound of Formula I:

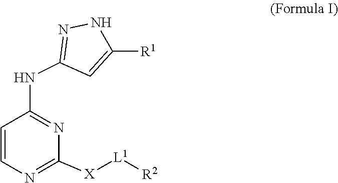

or a pharmaceutically acceptable salt, stereoisomer, and/or N-oxide thereof, wherein:
L¹ is a bond or C₁-C₆ alkylene;
X is NR^{A} or O;
R¹ is C₃-C₆ cycloalkyl;
R² is 5-10 membered heterocyclyl having at least one nitrogen, wherein the heterocyclyl is optionally substituted by one or two halo, oxo, hydroxyl, or C₁-C₄ alkyl; and
R^{A} is from H and C₁-C₆ alkyl;
wherein when R² is a 6-membered monocyclic heterocyclyl, R¹ is cyclopentyl.

In some embodiments, R¹ is cyclopropyl. In other embodiments, R¹ is cyclopenyl.

In some embodiments, R² is 5-6 membered heterocyclyl having at least one nitrogen. For example, R² is pyrrolidinyl or piperidinyl.

In other embodiments, R² is 6-10 membered spiroheterocycle having at least one nitrogen, 6-10 membered fused bicyclic heterocycle having at least one nitrogen, or 6-10 membered bridged heterocycle having at least one nitrogen, each of which is optionally substituted by one or two halo, oxo, hydroxyl, or C₁-C₄ alkyl.

In some embodiments, R² is 6-10 membered spiroheterocycle having at least one nitrogen, optionally substituted by one or two halo, oxo, hydroxyl, or C₁-C₄ alkyl.

In some embodiments, R² is 6-10 membered bridged heterocycle having at least one nitrogen, optionally substituted by one or two halo, oxo, hydroxyl, or C₁-C₄ alkyl.

In some embodiments, R² is 6-10 membered fused bicyclic heterocycle having at least one nitrogen, optionally substituted by one or two halo, oxo, hydroxyl, or C₁-C₄ alkyl.

In some embodiments, R^{A} is H. In other embodiments, R^{A} is C₁-C₆ alkyl such as methyl.

A contemplated compound of the present disclosure is selected from the group consisting of:

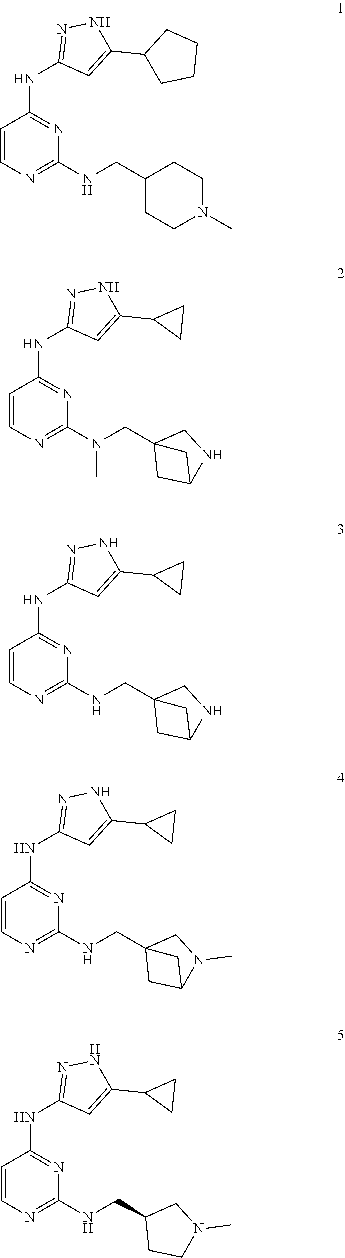 [SMILES: CN1CCC(CNc2nccc(Nc3cc(C4CCCC4)[nH]n3)n2)CC1 ; CN(CC12CNC(C1)C2)c1nccc(Nc2cc(C3CC3)[nH]n2)n1 ; c1cc(Nc2cc(C3CC3)[nH]n2)nc(NCC23CNC(C2)C3)n1 ; CN1CC2(CNc3nccc(Nc4cc(C5CC5)[nH]n4)n3)CC1C2 ; CN1CC[C@@H](CNc2nccc(Nc3cc(C4CC4)[nH]n3)n2)C1]

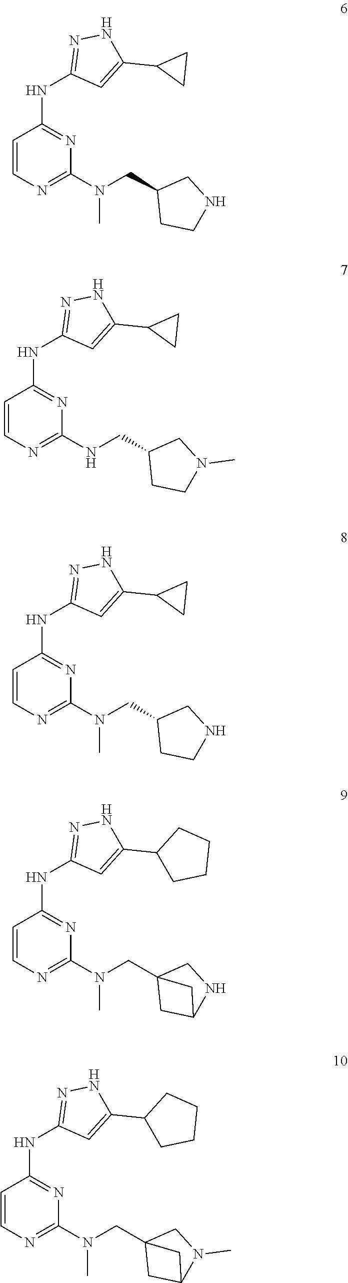 [SMILES: CN(C[C@@H]1CCNC1)c1nccc(Nc2cc(C3CC3)[nH]n2)n1 ; CN1CC[C@H](CNc2nccc(Nc3cc(C4CC4)[nH]n3)n2)C1 ; CN(C[C@H]1CCNC1)c1nccc(Nc2cc(C3CC3)[nH]n2)n1 ; CN(CC12CNC(C1)C2)c1nccc(Nc2cc(C3CCCC3)[nH]n2)n1 ; CN(CC12CC(C1)N(C)C2)c1nccc(Nc2cc(C3CCCC3)[nH]n2)n1]

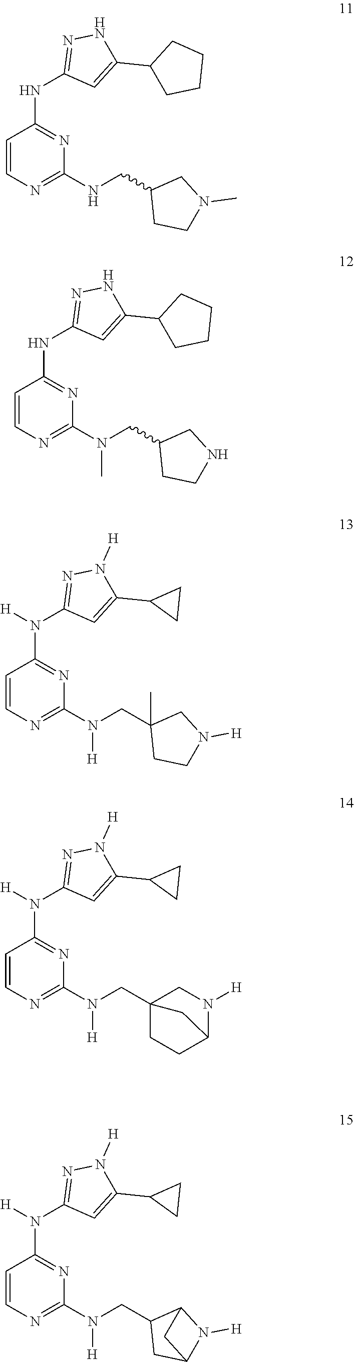 [SMILES: CN1CCC(CNc2nccc(Nc3cc(C4CCCC4)[nH]n3)n2)C1 ; CN(CC1CCNC1)c1nccc(Nc2cc(C3CCCC3)[nH]n2)n1 ; CC1(CNc2nccc(Nc3cc(C4CC4)[nH]n3)n2)CCNC1 ; c1cc(Nc2cc(C3CC3)[nH]n2)nc(NCC23CCC(C2)NC3)n1 ; c1cc(Nc2cc(C3CC3)[nH]n2)nc(NCC2CC3CC2N3)n1]

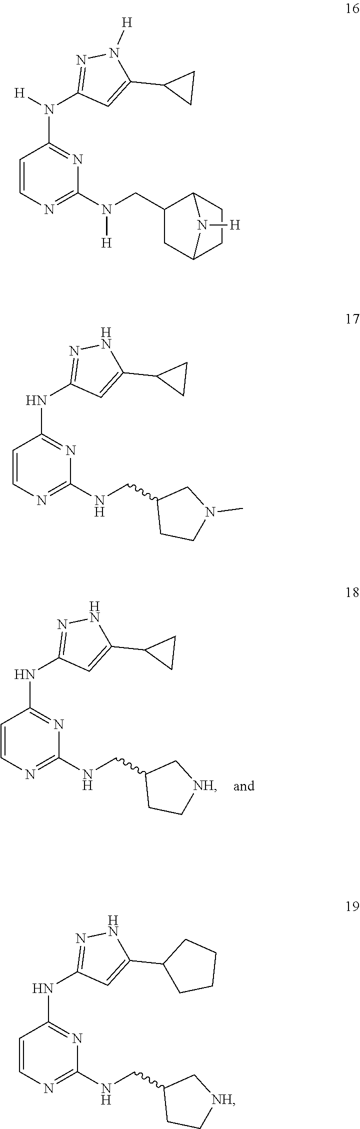 [SMILES: c1cc(Nc2cc(C3CC3)[nH]n2)nc(NCC2CC3CCC2N3)n1 ; CN1CCC(CNc2nccc(Nc3cc(C4CC4)[nH]n3)n2)C1 ; c1cc(Nc2cc(C3CC3)[nH]n2)nc(NCC2CCNC2)n1 ; "and" ; c1cc(Nc2cc(C3CCCC3)[nH]n2)nc(NCC2CCNC2)n1]

or a pharmaceutically acceptable salt, stereoisomer, and/or N-oxide thereof.

Also contemplated herein are following compounds:

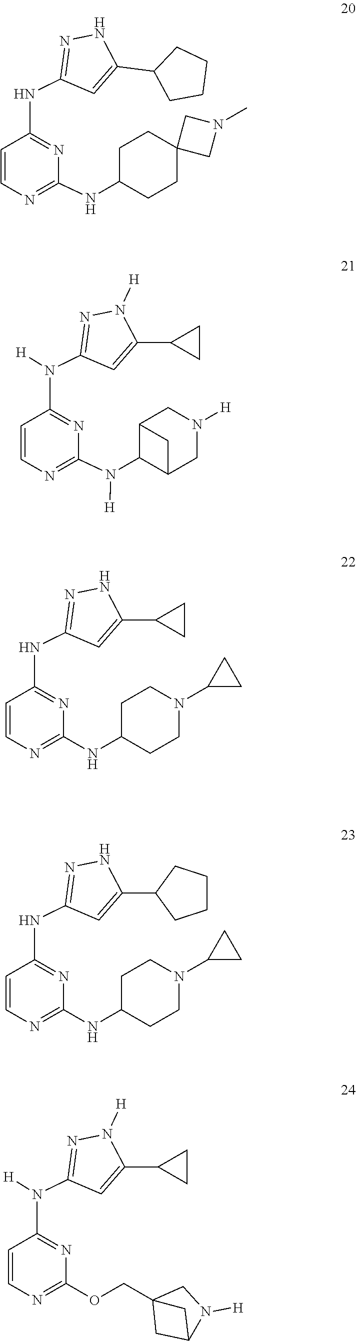 [SMILES: CN1CC2(CCC(Nc3nccc(Nc4cc(C5CCCC5)[nH]n4)n3)CC2)C1 ; c1cc(Nc2cc(C3CC3)[nH]n2)nc(NC2C3CNCC2C3)n1 ; c1cc(Nc2cc(C3CC3)[nH]n2)nc(NC2CCN(C3CC3)CC2)n1 ; c1cc(Nc2cc(C3CCCC3)[nH]n2)nc(NC2CCN(C3CC3)CC2)n1 ; c1cc(Nc2cc(C3CC3)[nH]n2)nc(OCC23CNC(C2)C3)n1]

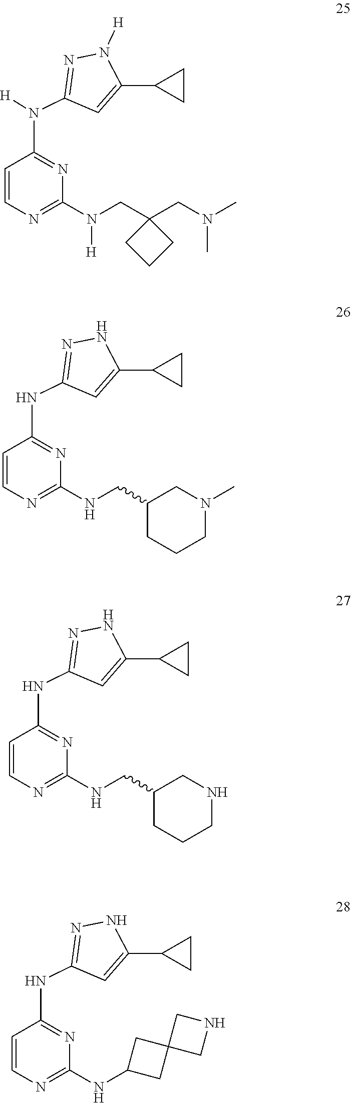 [SMILES: CN(C)CC1(CNc2nccc(Nc3cc(C4CC4)[nH]n3)n2)CCC1 ; CN1CCCC(CNc2nccc(Nc3cc(C4CC4)[nH]n3)n2)C1 ; c1cc(Nc2cc(C3CC3)[nH]n2)nc(NCC2CCCNC2)n1 ; c1cc(Nc2cc(C3CC3)[nH]n2)nc(NC2CC3(CNC3)C2)n1]

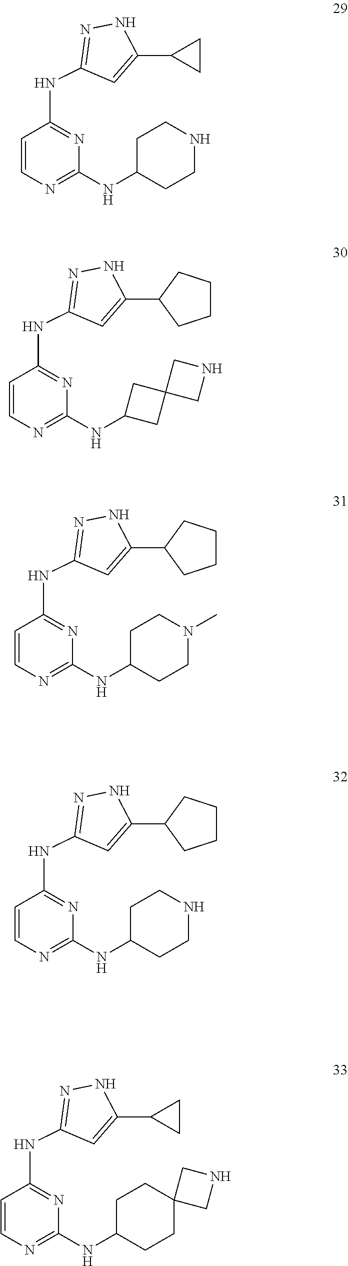 [SMILES: c1cc(Nc2cc(C3CC3)[nH]n2)nc(NC2CCNCC2)n1 ; c1cc(Nc2cc(C3CCCC3)[nH]n2)nc(NC2CC3(CNC3)C2)n1 ; CN1CCC(Nc2nccc(Nc3cc(C4CCCC4)[nH]n3)n2)CC1 ; c1cc(Nc2cc(C3CCCC3)[nH]n2)nc(NC2CCNCC2)n1 ; c1cc(Nc2cc(C3CC3)[nH]n2)nc(NC2CCC3(CC2)CNC3)n1]

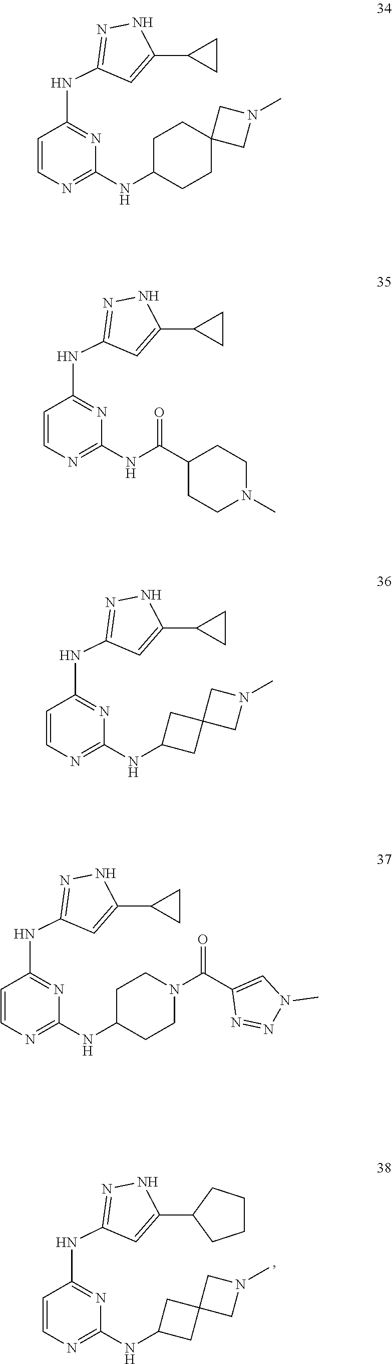 [SMILES: CN1CC2(CCC(Nc3nccc(Nc4cc(C5CC5)[nH]n4)n3)CC2)C1 ; CN1CCC(C(=O)Nc2nccc(Nc3cc(C4CC4)[nH]n3)n2)CC1 ; CN1CC2(CC(Nc3nccc(Nc4cc(C5CC5)[nH]n4)n3)C2)C1 ; Cn1cc(C(=O)N2CCC(Nc3nccc(Nc4cc(C5CC5)[nH]n4)n3)CC2)nn1 ; CN1CC2(CC(Nc3nccc(Nc4cc(C5CCCC5)[nH]n4)n3)C2)C1]

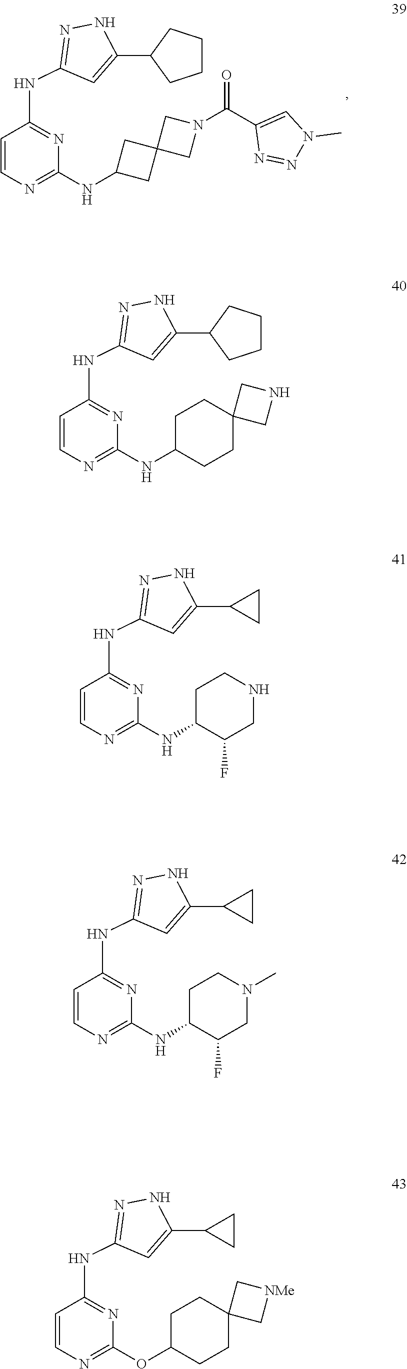 [SMILES: Cn1cc(C(=O)N2CC3(CC(Nc4nccc(Nc5cc(C6CCCC6)[nH]n5)n4)C3)C2)nn1 ; c1cc(Nc2cc(C3CCCC3)[nH]n2)nc(NC2CCC3(CC2)CNC3)n1 ; F[C@H]1CNCC[C@H]1Nc1nccc(Nc2cc(C3CC3)[nH]n2)n1 ; CN1CC[C@@H](Nc2nccc(Nc3cc(C4CC4)[nH]n3)n2)[C@@H](F)C1 ; CN1CC2(CCC(Oc3nccc(Nc4cc(C5CC5)[nH]n4)n3)CC2)C1]

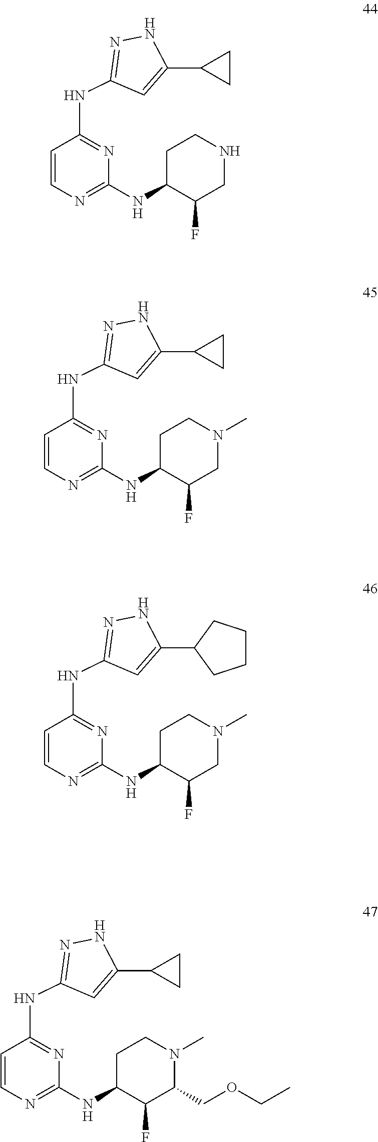 [SMILES: F[C@@H]1CNCC[C@@H]1Nc1nccc(Nc2cc(C3CC3)[nH]n2)n1 ; CN1CC[C@H](Nc2nccc(Nc3cc(C4CC4)[nH]n3)n2)[C@H](F)C1 ; CN1CC[C@H](Nc2nccc(Nc3cc(C4CCCC4)[nH]n3)n2)[C@H](F)C1 ; CCOC[C@@H]1[C@@H](F)[C@@H](Nc2nccc(Nc3cc(C4CC4)[nH]n3)n2)CCN1C]

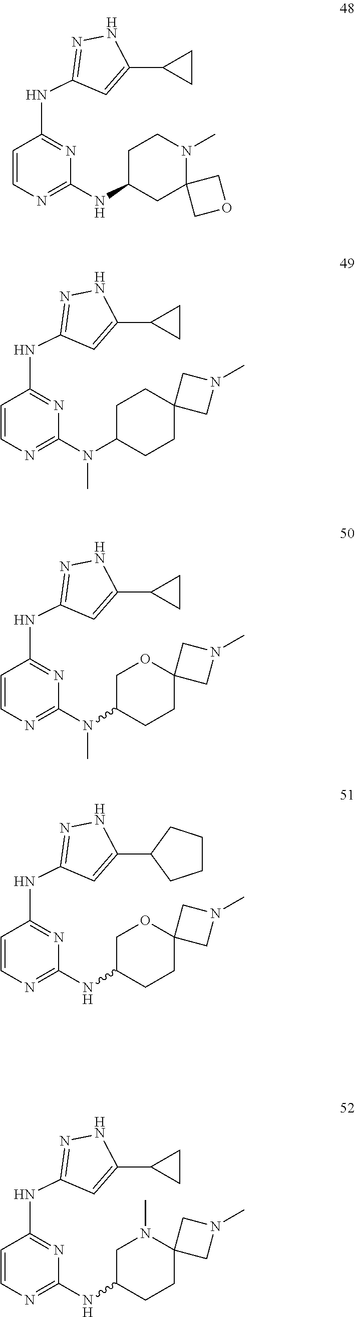 [SMILES: CN1CC[C@H](Nc2nccc(Nc3cc(C4CC4)[nH]n3)n2)CC12COC2 ; CN1CC2(CCC(N(C)c3nccc(Nc4cc(C5CC5)[nH]n4)n3)CC2)C1 ; CN1CC2(CCC(N(C)c3nccc(Nc4cc(C5CC5)[nH]n4)n3)CO2)C1 ; CN1CC2(CCC(Nc3nccc(Nc4cc(C5CCCC5)[nH]n4)n3)CO2)C1 ; CN1CC2(CCC(Nc3nccc(Nc4cc(C5CC5)[nH]n4)n3)CN2C)C1]

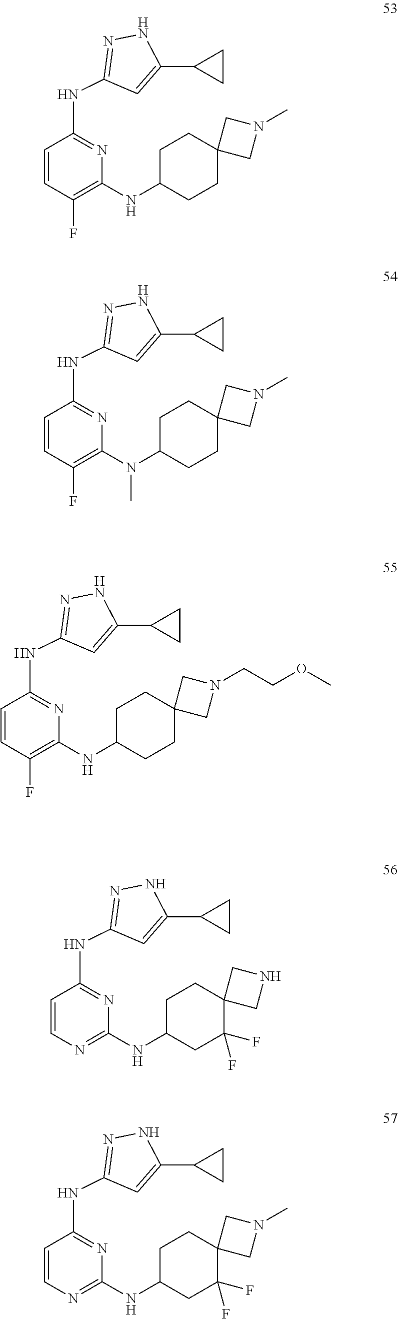 [SMILES: CN1CC2(CCC(Nc3nc(Nc4cc(C5CC5)[nH]n4)ccc3F)CC2)C1 ; CN1CC2(CCC(N(C)c3nc(Nc4cc(C5CC5)[nH]n4)ccc3F)CC2)C1 ; COCCN1CC2(CCC(Nc3nc(Nc4cc(C5CC5)[nH]n4)ccc3F)CC2)C1 ; FC1(F)CC(Nc2nccc(Nc3cc(C4CC4)[nH]n3)n2)CCC12CNC2 ; CN1CC2(CCC(Nc3nccc(Nc4cc(C5CC5)[nH]n4)n3)CC2(F)F)C1]

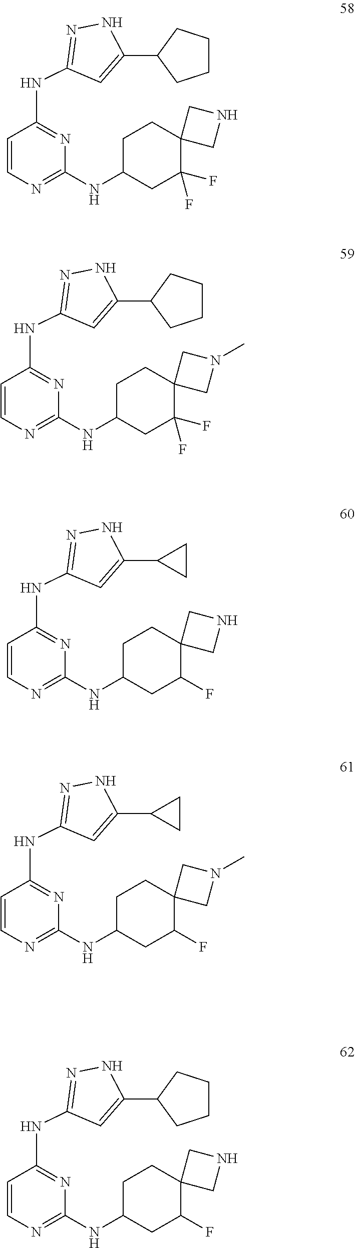 [SMILES: FC1(F)CC(Nc2nccc(Nc3cc(C4CCCC4)[nH]n3)n2)CCC12CNC2 ; CN1CC2(CCC(Nc3nccc(Nc4cc(C5CCCC5)[nH]n4)n3)CC2(F)F)C1 ; FC1CC(Nc2nccc(Nc3cc(C4CC4)[nH]n3)n2)CCC12CNC2 ; CN1CC2(CCC(Nc3nccc(Nc4cc(C5CC5)[nH]n4)n3)CC2F)C1 ; FC1CC(Nc2nccc(Nc3cc(C4CCCC4)[nH]n3)n2)CCC12CNC2]

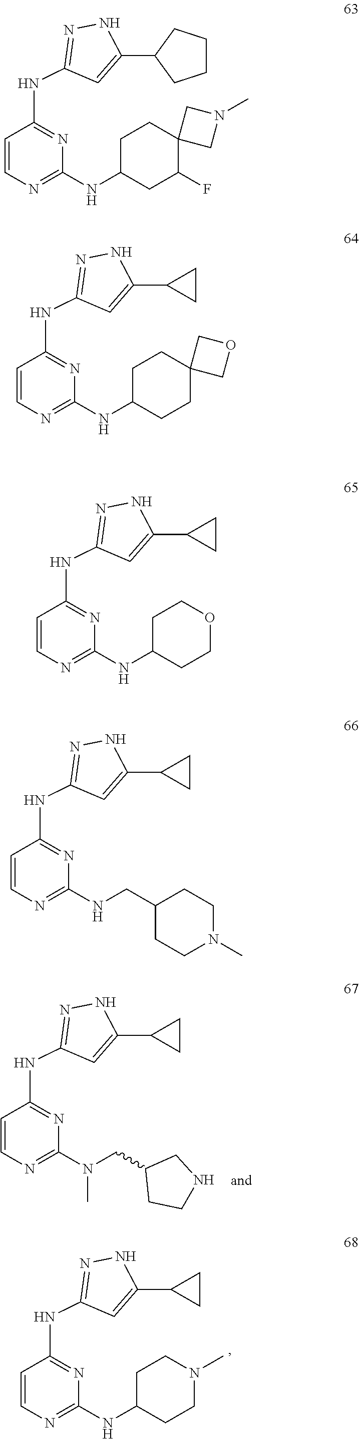

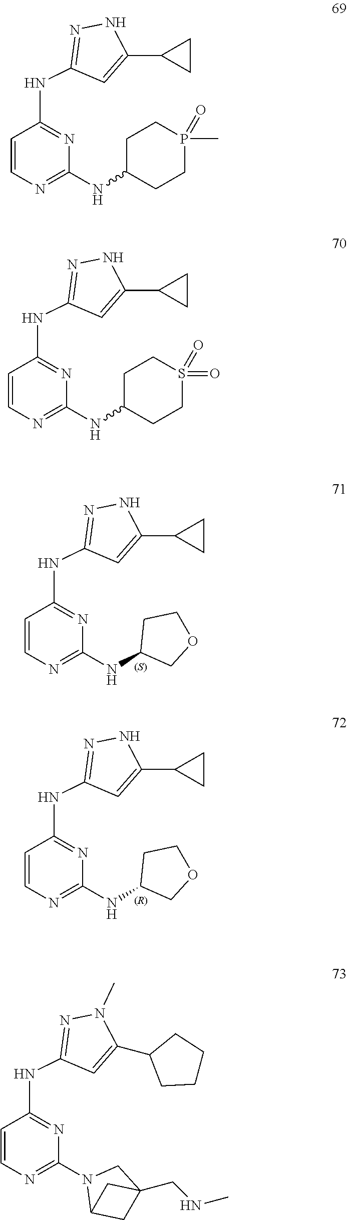

or a pharmaceutically acceptable salt, stereoisomer, and/or N-oxide thereof.

Disclosed compounds described herein may be present in a salt form, and the salt form of the compound is a pharmaceutically acceptable salt, and/or compounds described herein may be present in a prodrug form. Any convenient prodrug forms of the subject compounds can be prepared, for example, according to the strategies and methods described by Rautio et al. (“Prodrugs: design and clinical applications”, Nature Reviews Drug Discovery 7, 255-270 (February 2008)). Compounds described herein may be present in a solvate form.

In some embodiments, the compounds, or a prodrug form thereof, are provided in the form of pharmaceutically acceptable salts. Compounds containing an amine functional group or a nitrogen-containing heteroaryl group may be basic in nature and may react with any number of inorganic and organic acids to from the corresponding pharmaceutically acceptable salts. Inorganic acids commonly employed to form such salts include hydrochloric, hydrobromic, hydroiodic, sulfuric, and phosphoric acids, and related inorganic acids. Organic acids commonly employed to form such salts include para-toluenesulfonic, methanesulfonic, oxalic, para-bromophenylsulfonic, fumaric, maleic, carbonic, succinic, citric, benzoic and acetic acid, and related organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, O-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the related salts.

It is understood that all variations of salts, solvates, hydrates, prodrugs and stereoisomers are meant to be encompassed by the present disclosure.

### Pharmaceutical Compositions and Formulations

The compounds, prodrugs, and compositions described herein can be useful as pharmaceutical compositions for administration to a subject in need thereof.

Accordingly, pharmaceutical compositions are presented that can comprise at least a compound described herein, a pharmaceutically acceptable salt thereof, or a prodrug thereof, and at least one pharmaceutically acceptable carriers, diluent, stabilizers, excipients, dispersing agents, suspending agents, or thickening agents. For example, a disclosed pharmaceutical compositions may include one or more of the disclosed compounds, pharmaceutically acceptable salts, or prodrugs described herein. Contemplated compositions may include a compound, a pharmaceutically acceptable salt thereof, or a prodrug thereof in a therapeutically effective amount, for example, a disclosed pharmaceutical composition may be formulated for parenteral administration to a subject in need thereof, formulated for intravenous administration to a subject in need thereof, or formulated for subcutaneous administration to a subject in need thereof.

### Methods of Treatment

As described above, embodiments of the present disclosure include the use of compounds, prodrugs, and pharmaceutical compositions described herein to treat a Myc protein associated proliferative disease in a subject in need thereof. Such proliferative diseases include cancer, for example, a cancer selected from a group consisting of head and neck cancer, nervous system cancer, brain cancer, neuroblastoma, lung/mediastinum cancer, breast cancer, esophageal cancer, stomach cancer, liver cancer, biliary tract cancer, pancreatic cancer, small bowel cancer, large bowel cancer, colorectal cancer, gynecological cancer, genito-urinary cancer, ovarian cancer, thyroid gland cancer, adrenal gland cancer, skin cancer, melanoma, bone sarcoma, soft tissue sarcoma, pediatric malignancy, Hodgkin's disease, non-Hodgkin's lymphoma, myeloma, leukemia, and metastasis from an unknown primary site.

In some embodiments, a contemplated method of treating includes treating a cancer that is a Myc protein associated cancer, e.g., wherein the Myc protein is selected from the group consisting of a N-Myc protein, a c-MYc protein, a L-Myc protein, a human N-Myc protein, a human c-Myc protein, and a human L-Myc protein.

For example, provided herein is a method of treating a cancer selected from the group consisting of neuroblastoma, small cell lung carcinoma, breast cancer or a hematopoietic cancer.

In some embodiments, a disclosed method to treat cancer further comprises a second therapy, wherein the secondary therapy is an antineoplastic therapy, e.g., a contemplated method may further comprise administering an antineoplastic therapy such as one or more agents selected from a DNA topoisomerase I or II inhibitor, a DNA damaging agent, an immunotherapeutic agent (e.g., an antibody, cytokine, immune checkpoint inhibitor or cancer vaccine), an antimetabolite or a thymidylate synthase (TS) inhibitor, a microtubule targeted agent, ionizing radiation, an inhibitor of a mitosis regulator or a mitotic checkpoint regulator, an inhibitor of a DNA damage signal transducer, and an inhibitor of a DNA damage repair enzyme. For example, additional antineoplastic therapy may be selected from the group consisting of immunotherapy (e.g., immuno-oncologic therapy), radiation therapy, photodynamic therapy, gene-directed enzyme prodrug therapy (GDEPT), antibody-directed enzyme prodrug therapy (ADEPT), gene therapy, and controlled diets.

The present disclosure also contemplates the use of compounds, prodrugs, and pharmaceutical compositions described herein to modulate the amount and activity of a Myc protein (in vitro or in a patient), where the Myc protein may be for example a N-Myc protein, a c-MYc protein, a L-Myc protein, a human N-Myc protein, a human c-Myc protein, and/or a human L-Myc protein.

For example, the disclosure provides a method of modulating the amount (e.g., the concentration) and/or activity of a Myc protein such as (e.g. degrading a Myc protein, or modulating the rate of degradation of a Myc protein) that comprises contacting a Myc protein with an effective amount of a compound described herein, or a pharmaceutically acceptable salt, stereoisomer, and/or N-oxide thereof, including embodiments or from any examples, tables or figures.

Contemplated methods include methods of modulating the protein-protein interactions of the Myc family protein, or a method of decreasing the amount and decreasing the level of activity of a Myc protein.

A disclosed method of modulating the amount and activity of a Myc protein may include co-administering a compound described herein, or a pharmaceutically acceptable salt thereof, and a therapeutically effective amount of a second agent, e.g., therapeutic agent.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (i.e., amounts, temperatures, etc.), but some experimental error and deviation should, of course, be allowed for.

### General Experimental

¹H and ¹³C NMR spectra were recorded in CDCl₃ (residual internal standard CHCl₃=δ 7.26), DMSO-d₆ (residual internal standard CD₃SOCD₂H=δ 2.50), methanol-d₄ (residual internal standard CD₂HOD=δ 3.20), or acetone-d₆ (residual internal standard CD₃COCD₂H=δ 2.05). The chemical shifts (δ) reported are given in parts per million (ppm) and the coupling constants (J) are in Hertz (Hz). The spin multiplicities are reported as s=singlet, bs=broad singlet, bm=broad multiplet, d=doublet, t=triplet, q=quartet, p=pentuplet, dd=doublet of doublet, ddd=doublet of doublet of doublet, dt=doublet of triplet, td=triplet of doublet, tt=triplet of triplet, and m=multiplet.

UPLC-MS analysis was carried out on a Waters Acquity UPLC system consisting of an Acquity I-Class Sample Manager-FL, Acquity I-Class Binary Solvent Manager and an Acquity UPLC Column Manager. UV detection was afforded using an Acquity UPLC PDA detector (scanning from 210 to 400 nm), whilst mass detection was achieved using aa Acquity QDa detector (mass scanning from 100-1250 Da; positive and negative modes simultaneously). A Waters Acquity UPLC BEH C18 column (2.1×50 mm, 1.7 mm) was used to separate the analytes.

Samples were prepared by dissolution (with or without sonication) into 1 mL of 50% (v/v) MeCN in water. The resulting solutions were then filtered through a 0.2 mm syringe filter before submitting for analysis. All of the solvents, including formic acid and 36% ammonia solution, were purchased as the HPLC grade. Conditions (Acidic 2 min): 0.1% v/v Formic acid in water [Eluent A]; 0.1% v/v Formic acid in MeCN [Eluent B]; Flow rate 0.8 mL/min; injection volume 2 mL and 1.5 min equilibration time between samples. Conditions (Acidic 4 min): 0.1% v/v formic acid in water [Eluent A]; 0.1% v/v formic acid in MeCN [Eluent B]; Flow rate 0.8 mL/min; injection volume 2 mL and 1.5 min equilibration time between samples. Conditions (Basic 2 min): 0.1% ammonia in water [Eluent A]; 0.1% ammonia in MeCN [Eluent B]; Flow rate 0.8 mL/min; injection volume 2 mL and 1.5 min equilibration time between samples. Conditions (Basic 4 min): 0.1% ammonia in water [Eluent A]; 0.1% ammonia in MeCN [Eluent B]; Flow rate 0.8 mL/min; injection volume 2 mL and 1.5 min equilibration time between samples.

### General Synthetic Schemes

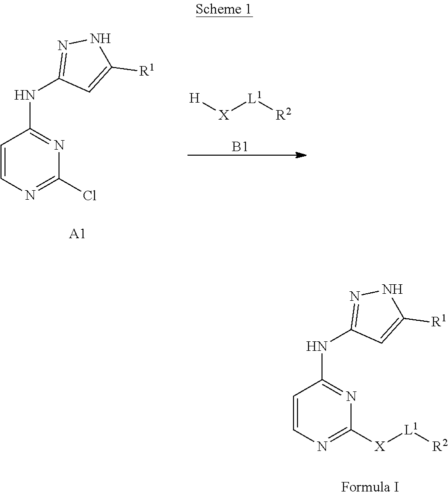

In certain embodiments, a compound of Formula I is prepared according to Scheme 1. Intermediate A1 (e.g., disclosed herein) is reacted with nucleophiles B1 (e.g., alcohols or amines disclosed herein) in the presence of a base (e.g., N,N-diisopropylethylamine) at elevated temperatures to produce a compound of Formula I. In certain embodiments, amine B1 may comprise a protecting group (e.g., tert-butyloxycarbonyl (Boc)). In certain embodiments, when amine B1 comprises a protecting group (e.g., tert-butyloxycarbonyl (Boc)), the intermediate produced from the reaction thereof with intermediate A1 in the presence of a base may further be deprotected (e.g., in the presence of reducing agents such as LiAlH₄ or acid such as TFA) to produce a compound of Formula I.

### Synthesis of Intermediates

### 2-Chloro-N-(5-cyclopropyl-1H-pyrazol-3-yl)pyrimidin-4-amine

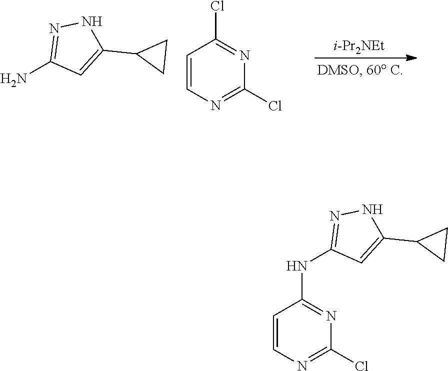

A solution of 5-cyclopropyl-1H-pyrazol-3-amine (4.55 g, 36.9 mmol), 2,4-dichloropyrimidine (5.00 g, 33.6 mmol) and N,N-diisopropylethylamine (8.8 mL, 50 mmol) in DMSO (50 mL) was heated to 60° C. and stirred for 16 hours. The reaction mixture was then cooled to ambient temperature, then added to ice water (ca. 1 L). The precipitate was collected by filtration, then dried under reduced pressure to afford the title compound as a solid (7.41 g, 31.4 mmol, 94%).

¹H NMR (400 MHz, DMSO-d₆) δ 12.17 (s, 1H), 10.26 (s, 1H), 8.14 (s, 1H), 7.31 (s, 1H), 5.93 (s, 1H), 1.89 (tt, J=8.7, 5.2 Hz, 1H), 1.01-0.88 (m, 2H), 0.68 (q, J=3.5, 1.9 Hz, 2H).

UPLC-MS (basic method, 2 min): Rt: 0.87 min, m/z: 236.2 [M+H]⁺.

### Synthesis of 2-Chloro-N-(5-cyclopentyl-1H-pyrazol-3-yl)pyrimidin-4-amine

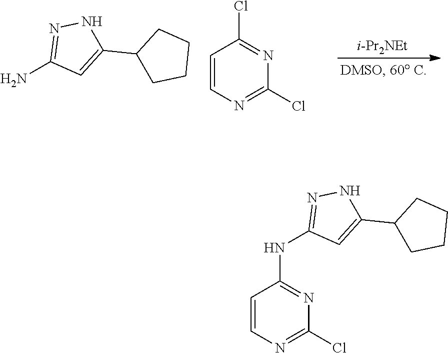

A solution of 2,4-dichloropyrimidine (450 mg, 3.0 mmol, 1.0 eq) in anhydrous DMSO (10.0 mL) was treated sequentially with 5-cyclopentyl-1H-pyrazol-3-amine (502 mg, 3.2 mmol, 1.10 eq) and N,N-diisopropylethylamine (0.79 mL, 4.5 mmol, 1.50 eq), and the resulting solution was stirred at 60° C. for 20 h. The reaction mixture was cooled to ambient temperature and poured into ice water, affording a suspension, which was stirred for 5 min. The mixture was filtered to afford a solid, which was washed with water (50 mL) before being dried under vacuum to constant weight to give the desired compound as a solid (675 mg, 2.56 mmol, 85%).

¹H NMR (400 MHz, DMSO) δ 12.19 (s, 1H), 10.31 (s, 1H), 8.16 (s, 1H), 3.02 (q, J=8.3 Hz, 1H), 2.05-1.94 (m, 2H), 1.74-1.66 (m, 2H), 1.58 (ddt, J=20.3, 15.7, 7.0 Hz, 4H).

UPLC-MS (Basic Method, 2 min): rt 0.98 min, m/z=263.3 [M+H]⁺.

### Example 1. Synthesis of Compound 1

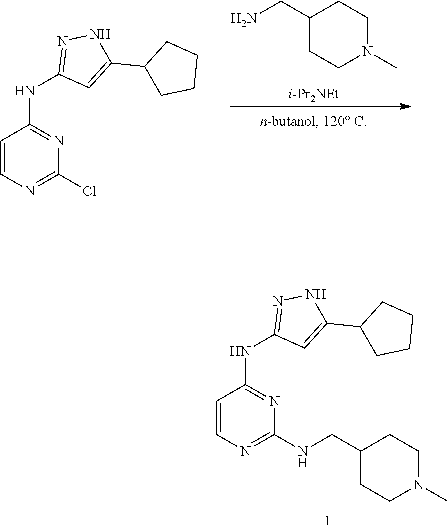

### tert-Butyl (2-(4-((5-Cyclopentyl-1H-pyrazol-3-yl)amino)pyrimidin-2-yl)-2-azaspiro[3.3]heptan-6-yl)carbamate

A solution of 2-chloro-N-(5-cyclopentyl-1H-pyrazol-3-yl)pyrimidin-4-amine (120 mg, 0.455 mmol), (1-methyl-4-piperidyl)methanamine (146 mg, 1.14 mmol), and N,N-diisopropylethylamine (0.277 mL, 1.59 mmol) in n-butanol (5 mL) was heated to 100° C. and stirred for 72 hours. The reaction mixture was then cooled to ambient temperature, and the volatiles were removed under reduced pressure. The residue was then loaded on to an SCX cartridge, the cartridge was washed with methanol. The compound was eluted from the SCX cartridge with 2 M NH₃ in methanol, then the eluent was concentrated to dryness under reduced pressure. The crude residue was purified by reversed-phase acidic prepHPLC, using TFA as the additive, followed by reversed-phase basic prepHPLC, using ammonia as the additive, to afford the title compound as a solid (58 mg, 0.16 mmol, 36%).

¹H NMR (400 MHz, DMSO-d₆) δ 11.87 (s, 1H), 9.28 (s, 1H), 8.11-7.37 (m, 1H), 6.79-6.02 (m, 3H), 3.13 (t, J=6.4 Hz, 2H), 3.03-2.92 (m, 1H), 2.72 (d, J=11.1 Hz, 2H), 2.11 (s, 3H), 1.98 (s, 2H), 1.84-1.44 (m, 12H), 1.27-1.03 (m, 2H).

¹H NMR (400 MHz, methanol-d₄) δ 7.77 (d, J=5.9 Hz, 1H), 6.34 (s, 1H), 6.11 (s, 1H), 3.25 (d, J=6.9 Hz, 2H), 3.11-3.02 (m, 1H), 2.89 (d, J=11.5 Hz, 2H), 2.26 (s, 3H), 2.13-1.95 (m, 4H), 1.89-1.62 (m, 9H), 1.39-1.23 (m, 2H).

UPLC-MS (basic method, 4 min): Rt: 1.37 min, m/z: 356.2 [M+H]⁺.

### Example 2. Synthesis of Compound 2

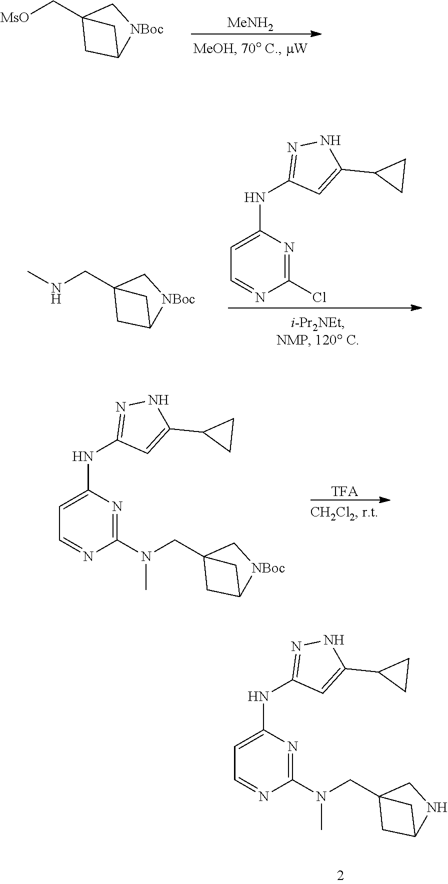

### tert-Butyl 4-((Methylamino)methyl)-2-azabicyclo[2.1.1]hexane-2-carboxylate

A solution of 2-methyl-2-propanyl 4-{[(methylsulfonyl)oxy]methyl}-2-azabicyclo[2.1.1]hexane-2-carboxylate (1.48 g, 5.08 mmol) in a 2.0 M solution of methylamine in MeOH (68 mL) was heated to 70° C. in a microwave reactor and stirred for 2 hours. The reaction mixture was concentrated to dryness under reduced pressure then the residue was diluted with a 0.5 M aqueous solution of NaOH (100 mL), then extracted with EtOAc (3×100 mL). The combined organic extracts were dried over Na₂SO₄, then concentrated to dryness under reduced pressure, to afford the title compound as an oil (820 mg, 3.62 mmol, 71%).

¹H NMR (400 MHz, chloroform-d) δ 4.34 (s, 1H), 3.23 (s, 2H), 2.83 (s, 2H), 2.47 (s, 3H), 1.78-1.70 (m, 2H), 1.54-1.38 (m, 11H).

UPLC-MS (basic method, 2 min): Rt: 0.92 min, m/z=227.1 [M+H]⁺.

### tert-Butyl 4-(((4-((5-Cyclopropyl-1H-pyrazol-3-yl)amino)pyrimidin-2-yl)(methyl)amino)methyl)-2-azabicyclo[2.1.1]hexane-2-carboxylate

A solution of 2-chloro-N-(5-cyclopropyl-1H-pyrazol-3-yl)pyrimidin-4-amine (174 mg, 0.736 mmol), tert-butyl 4-((methylamino)methyl)-2-azabicyclo[2.1.1]hexane-2-carboxylate (200 mg, 0.884 mmol) and N,N-diisopropylethylamine (0.28 mL, 1.6 mmol) in NMP (2 mL) was heated to 120° C. in a sealed tube and stirred for 78 hours. The reaction mixture was cooled to ambient temperature, then added dropwise to stirring ice water (100 mL), resulting in the formation of a precipitate. The precipitate was collected by filtration, washed with water (2×10 mL) then dried under reduced pressure to afford the title compound as a solid (238 mg, 0.559 mmol, 76%).

¹H NMR (400 MHz, DMSO-d₆) δ 11.94 (s, 1H), 9.34 (s, 1H), 7.84 (d, J=5.6 Hz, 1H), 6.26-6.03 (m, 2H), 4.14 (s, 1H), 3.88 (s, 2H), 3.12-3.00 (m, 5H), 1.89-1.80 (m, 1H), 1.79-1.75 (m, 2H), 1.44-1.29 (m, 11H), 0.97-0.88 (m, 2H), 0.66-0.60 (m, 2H).

¹H NMR (400 MHz, methanol-d₄) δ 7.85 (d, J=5.9 Hz, 1H), 6.29-5.96 (m, 2H), 4.25 (s, 1H), 3.93 (s, 2H), 3.18 (s, 2H), 3.14 (s, 3H), 1.95-1.78 (m, 3H), 1.50-1.34 (m, 11H), 1.01-0.94 (m, 2H), 0.75-0.66 (m, 2H).

UPLC-MS (basic method, 4 min): Rt: 1.75 min, m/z=426.3 [M+H]⁺.

### N²-((2-Azabicyclo[2.1.1]hexan-4-yl)methyl)-N⁴-(5-cyclopropyl-1H-pyrazol-3-yl)-N²-methylpyrimidine-2,4-diamine

To a solution of tert-butyl 4-(((4-((5-cyclopropyl-1H-pyrazol-3-yl)amino)pyrimidin-2-yl)(methyl)amino)methyl)-2-azabicyclo[2.1.1]hexane-2-carboxylate (70 mg, 0.17 mmol) in CH₂Cl₂ (2 mL) was added trifluoroacetic acid (2.0 mL, 26 mmol) and the reaction mixture was stirred at ambient temperature for 2 hour, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The residue was purified by reversed-phase basic prepHPLC to afford the title compound as a solid (16 mg, 47 mol, 29%).

¹H NMR (400 MHz, DMSO-d₆) δ 11.91 (s, 1H), 9.31 (s, 1H), 7.82 (d, J=5.6 Hz, 1H), 6.29-5.99 (m, 2H), 3.88 (s, 2H), 3.43 (s, 1H), 3.06 (s, 3H), 2.59 (s, 2H), 1.89-1.80 (m, 1H), 1.54-1.49 (m, 2H), 1.22-1.16 (m, 2H), 0.96-0.88 (m, 2H), 0.68-0.59 (m, 2H).

¹H NMR (400 MHz, methanol-d₄) δ 7.85 (d, J=5.9 Hz, 1H), 6.16-6.05 (m, 2H), 3.96 (s, 2H), 3.67 (s, 1H), 3.15 (s, 3H), 2.82 (s, 2H), 1.95-1.84 (m, 1H), 1.78-1.72 (m, 2H), 1.44-1.34 (m, 2H), 1.01-0.92 (m, 2H), 0.76-0.67 (m, 2H).

UPLC-MS (basic method, 4 min): Rt: 1.10 min, m/z=326.3 [M+H]⁺.

### Example 3. Synthesis of Compound 3

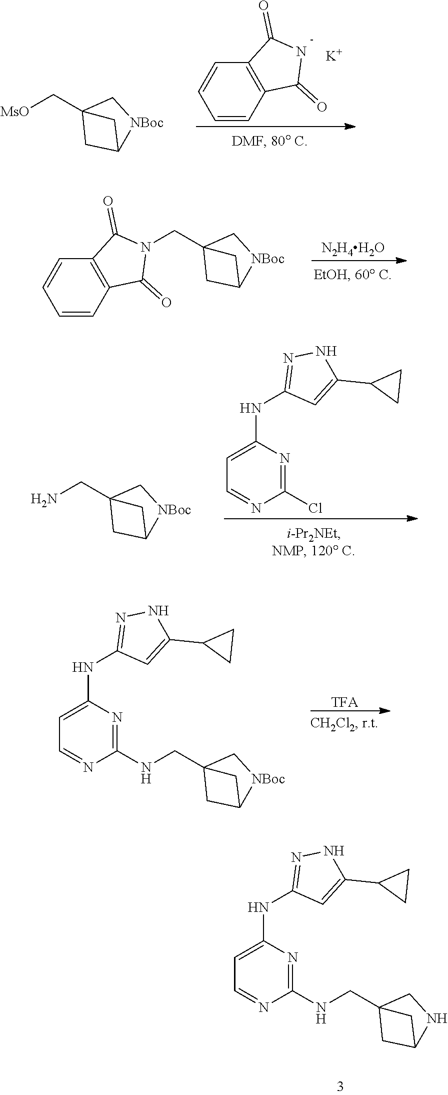

### tert-Butyl 4-((1,3-Dioxoisoindolin-2-yl)methyl)-2-azabicyclo[2.1.1]hexane-2-carboxylate

A solution of 2-methyl-2-propanyl 4-{[(methylsulfonyl)oxy]methyl}-2-azabicyclo[2.1.1]hexane-2-carboxylate (300 mg, 1.03 mmol) and potassium phthalimide (229 mg, 1.24 mmol) in DMF (15 mL) was heated to 80° C. and stirred for 4 hours. The reaction mixture was cooled to room temperature, then added dropwise to stirring ice water (250 mL) forming a precipitate. The precipitate was collected by filtration, then dried under suction for one hour, to afford the title compound as a solid (330 mg, 0.964 mmol, 94%).

¹H NMR (400 MHz, chloroform-d) δ 7.89-7.81 (m, 2H), 7.77-7.70 (m, 2H), 4.29 (s, 1H), 3.97 (s, 2H), 3.23 (s, 2H), 1.81-1.75 (m, 2H), 1.55-1.48 (m, 2H), 1.44 (s, 9H).

UPLC-MS (basic method, 2 min): Rt: 1.14 min, m/z: diagnostic m/z not observed.

### tert-Butyl 4-(Aminomethyl)-2-azabicyclo[2.1.1]hexane-2-carboxylate

A solution of hydrazine hydrate (139 μL, 1.42 mmol) and tert-butyl 4-((1,3-dioxoisoindolin-2-yl)methyl)-2-azabicyclo[2.1.1]hexane-2-carboxylate (325 mg, 0.949 mmol) in ethanol (7 mL) was heated to 60° C. and stirred for three hours. The reaction mixture was cooled to room temperature, then filtered through a pad of celite. The filtrate was then concentrated to dryness under reduced pressure, suspended in CH₂Cl₂ (20 mL), then filtered through a pad of celite. The filtrate was then concentrated to dryness under reduced pressure to afford the title compound as an oil (190 mg, 0.895 mmol, 94%).

¹H NMR (400 MHz, chloroform-d) δ 4.34 (s, 1H), 3.21 (s, 2H), 2.95 (s, 2H), 1.75-1.70 (m, 2H), 1.47 (d, J=1.3 Hz, 9H), 1.45-1.41 (m, 2H).

UPLC-MS (basic method, 2 min): product could not be visualized using a UV or ELS detector; m/z: diagnostic m/z not observed.

### tert-Butyl 4-(((4-((5-Cyclopropyl-1H-pyrazol-3-yl)amino)pyrimidin-2-yl)amino)methyl)-2-azabicyclo[2.1.1]hexane-2-carboxylate

A solution of 2-chloro-N-(5-cyclopropyl-1H-pyrazol-3-yl)pyrimidin-4-amine (174 mg, 0.736 mmol), tert-butyl 4-(aminomethyl)-2-azabicyclo[2.1.1]hexane-2-carboxylate (188 mg, 0.884 mmol) and N,N-diisopropylethylamine (0.28 mL, 1.6 mmol) in NMP (2 mL) was heated to 120° C. in a sealed tube and stirred for 144 hours. The reaction mixture was then cooled to ambient temperature, then added dropwise to stirring ice water (100 mL), resulting in the formation of a precipitate. The precipitate was collected by filtration, washed with water (2×10 mL) then dried under reduced pressure. The residue was purified by normal-phase column chromatography, over silica gel, eluting with a gradient of CH₂Cl₂ to 3:17 MeOH:CH₂Cl₂ to afford the title compound as a solid (146 mg, 0.355 mmol, 48%).

¹H NMR (400 MHz, DMSO-d₆) δ 11.68 (s, 1H), 8.74 (s, 1H), 7.81 (d, J=5.7 Hz, 1H), 6.45-5.93 (m, 3H), 4.17 (t, J=1.8 Hz, 1H), 3.61 (d, J=6.2 Hz, 2H), 3.16 (s, 2H), 1.90-1.81 (m, 1H), 1.80-1.71 (m, 2H), 1.41 (s, 9H), 1.38-1.31 (m, 2H), 0.96-0.82 (m, 2H), 0.72-0.64 (m, 2H).

UPLC-MS (basic method, 2 min): Rt: 1.01 min, m/z=412.5 [M+H]⁺.

### N²-((2-Azabicyclo[2.1.1]hexan-4-yl)methyl)-N⁴-(5-cyclopropyl-1H-pyrazol-3-yl)pyrimidine-2,4-diamine

To a solution of tert-butyl 4-(((4-((5-cyclopropyl-1H-pyrazol-3-yl)amino)pyrimidin-2-yl)amino)methyl)-2-azabicyclo[2.1.1]hexane-2-carboxylate (60 mg, 0.15 mmol) in CH₂Cl₂ (2 mL) was added trifluoroacetic acid (2.0 mL, 26 mmol) and the reaction mixture was stirred at ambient temperature for 1 hour, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The residue was purified by reversed-phase basic prepHPLC to afford the title compound as a solid (25 mg, 80 mol, 55%).

¹H NMR (400 MHz, DMSO-d₆) δ 11.91 (s, 1H), 9.25 (s, 1H), 7.77 (s, 1H), 6.66 (s, 1H), 6.35-5.87 (m, 2H), 4.03 (s, 2H), 3.57 (s, 1H), 3.55 (s, 1H), 2.67 (s, 2H), 1.88-1.78 (m, 1H), 1.55-1.51 (m, 2H), 1.19-1.14 (m, 2H), 0.91-0.87 (m, 2H), 0.68-0.62 (m, 2H)

¹H NMR (400 MHz, methanol-d₄) δ 7.79 (d, J=5.9 Hz, 1H), 6.11 (d, J=5.9 Hz, 1H), 6.00 (s, 1H), 3.71-3.66 (m, 3H), 2.87 (s, 2H), 1.90 (tt, J=8.5, 5.1 Hz, 1H), 1.78-1.72 (m, 2H), 1.36 (dd, J=4.8, 2.0 Hz, 2H), 1.01-0.91 (m, 2H), 0.78-0.67 (m, 2H);

UPLC-MS (basic method, 4 min): Rt: 0.95 min, m/z=312.1 [M+H]⁺.

### Example 4. Synthesis of Compound 4

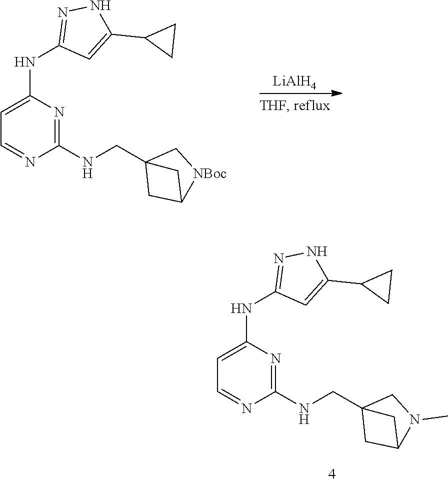 [SMILES: CC(C)(C)OC(=O)N1CC2(CNc3nccc(Nc4cc(C5CC5)[nH]n4)n3)CC1C2 ; "LiAlH4" ; CN1CC2(CNc3nccc(Nc4cc(C5CC5)[nH]n4)n3)CC1C2]

### N⁴-(5-Cyclopropyl-1H-pyrazol-3-yl)-N²-((2-methyl-2-azabicyclo[2.1.1]hexan-4-yl)methyl)pyrimidine-2,4-diamine

To a solution of tert-butyl 4-(((4-((5-cyclopropyl-1H-pyrazol-3-yl)amino)pyrimidin-2-yl)amino)methyl)-2-azabicyclo[2.1.1]hexane-2-carboxylate (80 mg, 0.19 mmol) in anhydrous THF (15 mL) was added a 2.4 M solution of lithium aluminum hydride in THF (0.81 mL, 1.9 mmol) dropwise at 0° C. The reaction mixture was heated to reflux and stirred for 7 hours. The reaction mixture was then cooled to 0° C., then quenched with water (100 L), then a 15% w/v sodium hydroxide solution (100 L), followed by water (300 L). The reaction mixture was warmed to ambient temperature and stirred for 10 minutes, after which magnesium sulfate (ca. 1 g) was added and the reaction mixture was stirred for a further 10 minutes. The reaction mixture was passed through a pad of celite, and the filter cake was washed with EtOAc (20 mL). The eluent was then concentrated to dryness under reduced pressure. The residue was purified by reversed-phase acidic prepHPLC, using TFA as a modifier, followed by reversed-phase basic prepHPLC, using ammonia as a modifier, to afford the title compound as a solid (8.0 mg, 0.399 25 mol, 13%).

¹H NMR (400 MHz, methanol-d₄) δ 7.79 (d, J=5.9 Hz, 1H), 6.22-6.01 (m, 2H), 3.61 (s, 2H), 3.38 (s, 1H), 2.66 (s, 2H), 2.47 (s, 3H), 1.94-1.84 (m, 1H), 1.71-1.67 (m, 2H), 1.62-1.58 (m, 2H), 1.00-0.94 (m, 2H), 0.75-0.69 (m, 2H).

UPLC-MS (basic method, 4 min): Rt: 0.99 min, m/z: 326.1 [M+H]⁺.

### Example 5. Synthesis of Compound 28

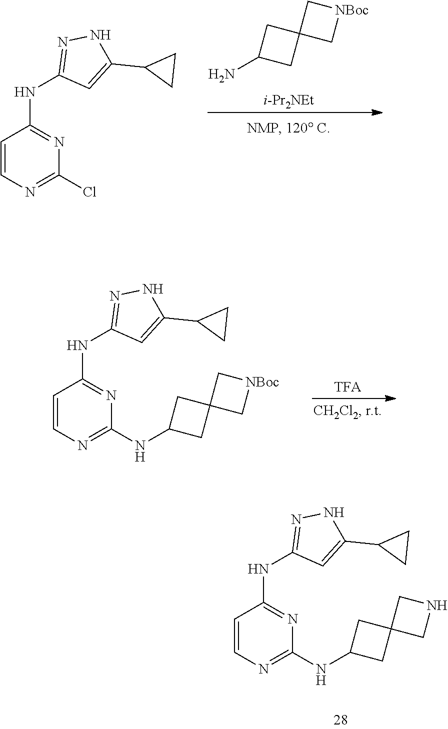 [SMILES: "i-Pr2NEt" ; Clc1nccc(Nc2cc(C3CC3)[nH]n2)n1 ; CC(C)(C)OC(=O)N1CC2(CC(N)C2)C1 ; CC(C)(C)OC(=O)N1CC2(CC(Nc3nccc(Nc4cc(C5CC5)[nH]n4)n3)C2)C1 ; c1cc(Nc2cc(C3CC3)[nH]n2)nc(NC2CC3(CNC3)C2)n1]

### Synthesis of tert-Butyl 6-((4-((5-cyclopropyl-1H-pyrazol-3-yl)amino)pyrimidin-2-yl)amino)-2-azaspiro[3.3]heptane-2-carboxylate

A solution of 2-chloro-N-(5-cyclopropyl-1H-pyrazol-3-yl)pyrimidin-4-amine (2.00 g, 8.49 mmol), tert-butyl 6-amino-2-azaspiro[3.3]heptane-2-carboxylate (3.60 g, 17.0 mmol) and N,N-diisopropylethylamine (4.43 mL, 25.5 mmol) in NMP (20 mL) was heated to 120° C. and stirred for 96 hours. The reaction mixture was then cooled to ambient temperature, then diluted with water (100 mL), then extracted with EtOAc (3×100 mL) The combined organic extracts were dried over Na₂SO₄ then concentrated to dryness under reduced pressure. The residue was purified by normal-phase column chromatography, over silica gel, eluting with a gradient of CH₂Cl₂ to 1:9 2 M NH₃ in MeOH:CH₂Cl₂ to afford the title compound as a solid (3.32 g, 8.07 mmol, 95%)

¹H NMR (400 MHz, DMSO-d₆) δ 11.91 (s, 1H), 9.28 (s, 1H), 7.76 (s, 1H), 6.84 (s, 1H), 6.41-5.95 (m, 2H), 4.25-4.09 (m, 1H), 3.91 (s, 2H), 3.77 (s, 2H), 2.49-2.42 (m, 2H), 2.11-2.02 (m, 2H), 1.92-1.80 (m, 1H), 1.37 (s, 9H), 0.98-0.87 (m, 2H), 0.73-0.60 (m, 2H).

UPLC-MS (basic method, 2 min): Rt: 1.02 min, m/z: 412.4 [M+H]⁺.

### N⁴-(5-cyclopropyl-1H-pyrazol-3-yl)-N²-(2-azaspiro[3.3]heptan-6-yl)pyrimidine-2,4-diamine

To a solution of tert-butyl 6-((4-((5-cyclopropyl-1H-pyrazol-3-yl)amino)pyrimidin-2-yl)amino)-2-azaspiro[3.3]heptane-2-carboxylate (650 mg, 1.58 mmol) in CH₂Cl₂ (5 mL) was added trifluoroacetic acid (5.0 mL, 65 mmol) and the reaction mixture was stirred at ambient temperature for 15 minutes, after which the reaction mixture was concentrated to dryness under reduced pressure. The crude residue was diluted with a 1 M aqueous solution of NaOH (30 mL), then extracted with CH₂Cl₂ (3×30 mL), followed by 3:1 CHCl₃:i-PrOH (3×50 mL). The combined organic extracts were dried over anhydrous Na₂SO₄, then concentrated to dryness under reduced pressure. The crude residue was purified by reversed-phase acidic prepHPLC, using TFA as the additive, followed by reversed-phase basic prepHPLC, using ammonia as the additive to afford the title compound as a solid (104 mg, 0.33 mmol, 21%).

¹H NMR (400 MHz, DMSO-d₆) δ 11.75 (s, 1H), 8.98 (s, 1H), 7.78 (d, J=5.7 Hz, 1H), 6.50 (s, 1H), 6.23-5.87 (m, 2H), 4.17 (apparent td, J=8.0, 7.1 Hz, 1H), 3.54 (s, 2H), 3.42 (s, 2H), 2.48-2.43 (m, 1H), 2.02 (apparent td, J=8.7, 2.9 Hz, 2H), 1.86 (tt, J=8.4, 5.1 Hz, 1H), 0.94-0.86 (m, 2H), 0.70-0.63 (m, 2H).

¹H NMR (400 MHz, methanol-d₄) δ 7.78 (d, J=5.9 Hz, 1H), 6.32-5.66 (m, 2H), 4.20 (p, J=8.0 Hz, 1H), 3.75 (s, 2H), 3.61 (s, 2H), 2.70-2.61 (m, 2H), 2.14-2.04 (m, 2H), 1.89 (tt, J=8.5, 5.0 Hz, 1H), 1.00-0.92 (m, 2H), 0.79-0.65 (m, 2H).

UPLC-MS (basic method, 4 min): Rt: 1.04 min, m/z: 312.1 [M+H]⁺.

### Example 6. Synthesis of Compound 29

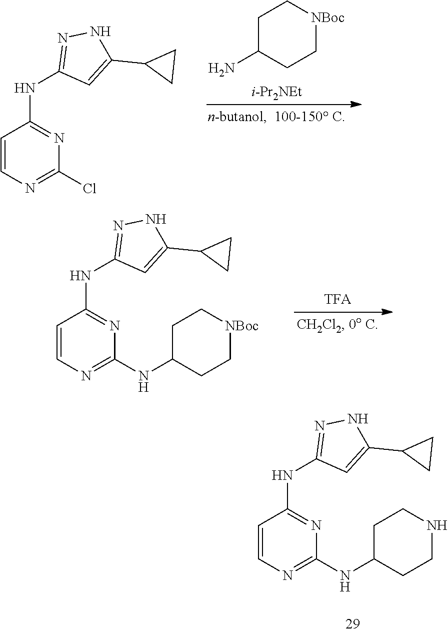 [SMILES: "i-Pr2NEt" ; Clc1nccc(Nc2cc(C3CC3)[nH]n2)n1 ; CC(C)(C)OC(=O)N1CCC(N)CC1 ; CC(C)(C)OC(=O)N1CCC(Nc2nccc(Nc3cc(C4CC4)[nH]n3)n2)CC1 ; c1cc(Nc2cc(C3CC3)[nH]n2)nc(NC2CCNCC2)n1]

### tert-Butyl 4-((4-((5-Cyclopropyl-1H-pyrazol-3-yl)amino)pyrimidin-2-yl)amino)piperidine-1-carboxylate

A solution of 2-chloro-N-(5-cyclopropyl-1H-pyrazol-3-yl)pyrimidin-4-amine (300 mg, 1.27 mmol), 4-amino-1-Boc-piperidine (637 mg, 3.18 mmol), and N,N-diisopropylethylamine (0.776 mL, 4.46 mmol) in n-butanol (15 mL) was heated to 100° C. and stirred for 18 hours. The reaction mixture was then heated to 110° C. and stirred for 16 hours. The reaction mixture was then heated to reflux and stirred for a further 72 hours. The reaction mixture was then heated 150° C. in a microwave reactor and stirred for 5 hours. The reaction mixture was then cooled to ambient temperature, and then concentrated to dryness under reduced pressure. The residue was purified by normal-phase column chromatography, over silica gel, eluting with a gradient of CH₂Cl₂ to 1:9 2 M NH₃ in MeOH:CH₂Cl₂ to afford the title compound as an oil (366 mg, 0.916 mmol, 72%).

¹H NMR (400 MHz, DMSO-d₆) δ 11.65 (s, 1H), 8.85 (s, 1H), 7.81 (d, J=5.7 Hz, 1H), 6.35-5.90 (m, 2H), 3.96-3.86 (m, 4H), 1.94-1.82 (m, 4H), 1.46-1.41 (m, 11H), 0.94-0.85 (m, 2H), 0.71-0.65 (m, 2H).

UPLC-MS (basic method, 2 min): Rt: 1.02 min, m/z: 400.4 [M+H]⁺.

### N⁴-(5-cyclopentyl-1H-pyrazol-3-yl)-N²-(piperidin-4-yl)pyrimidine-2,4-diamine

To a solution of tert-butyl 4-((4-((5-cyclopropyl-1H-pyrazol-3-yl)amino)pyrimidin-2-yl)amino)piperidine-1-carboxylate (292 mg, 0.250 mmol) in CH₂Cl₂ (2.5 mL) was added trifluoroacetic acid (2.5 mL, 23 mmol) at 0° C. and the reaction mixture was stirred for 1 hour, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The residue was purified by reversed-phase acidic prepHPLC, using TFA as the additive, followed by reversed-phase basic prepHPLC, using ammonia as the additive, to afford the title compound as a solid (105 mg, 0.351 mmol, 48%).

¹H NMR (400 MHz, methanol-d₄) δ 7.79 (d, J=6.0 Hz, 1H), 6.28-5.90 (m, 2H), 3.92-3.76 (m, 1H), 3.15-3.05 (m, 2H), 2.77-2.67 (m, 2H), 2.03 (d, J=12.7 Hz, 2H), 1.89 (tt, J=8.9, 5.1 Hz, 1H), 1.46 (qd, J=12.0, 3.9 Hz, 2H), 1.02-0.90 (m, 2H), 0.77-0.67 (m, 2H).

UPLC-MS (basic method, 4 min): Rt: 0.96 min, m/z: 300.1 [M+H]⁺.

### Synthesis of Compound 29

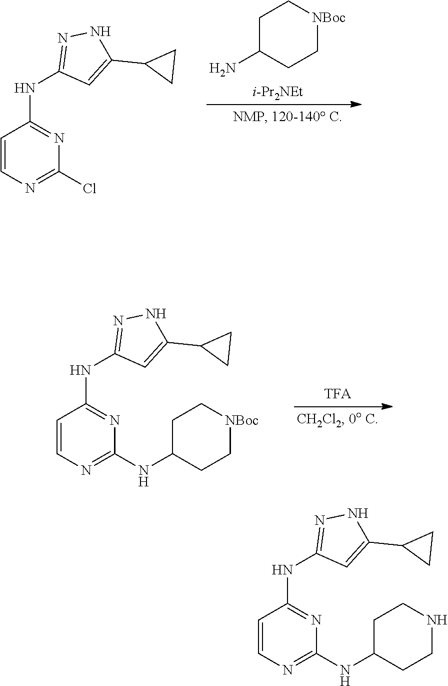 [SMILES: "i-Pr2NEt" ; Clc1nccc(Nc2cc(C3CC3)[nH]n2)n1 ; CC(C)(C)OC(=O)N1CCC(N)CC1 ; CC(C)(C)OC(=O)N1CCC(Nc2nccc(Nc3cc(C4CC4)[nH]n3)n2)CC1 ; c1cc(Nc2cc(C3CC3)[nH]n2)nc(NC2CCNCC2)n1]

### tert-Butyl 4-((4-((5-Cyclopropyl-1H-pyrazol-3-yl)amino)pyrimidin-2-yl)amino)piperidine-1-carboxylate

A solution of 2-chloro-N-(5-cyclopropyl-1H-pyrazol-3-yl)pyrimidin-4-amine (1.00 g, 4.24 mmol), 4-amino-1-Boc-piperidine (1.27 g, 6.36 mmol), and N,N-diisopropylethylamine (1.85 mL, 10.6 mmol) in NMP (10 mL) was heated to 120° C. and stirred for 24 hours. The reaction mixture was then heated to 130° C. and heated for a further 24 hours. The reaction mixture was then heated to 140° C. and heated for a further 24 hours. The reaction mixture was then cooled to ambient temperature, then added dropwise to ice water (ca. 250 mg) resulting in precipitation. The precipitate was collected by filtration, then dried under reduced pressure. The residue was purified by automated normal-phase column chromatography, over silica gel, eluting with a gradient of CH₂Cl₂ to 1:9 2 M NH₃ in MeOH:CH₂Cl₂ to afford the title compound as a solid (1.02 g, 2.55 mmol, 60%).

¹H NMR (400 MHz, DMSO-d₆) δ 11.89 (s, 1H), 9.26 (s, 1H), 7.77 (s, 1H), 6.51 (s, 1H), 6.28-6.10 (m, 2H), 3.97-3.79 (m, 3H), 3.03-2.69 (m, 2H), 2.03-1.71 (m, 3H), 1.41 (s, 9H), 1.36-1.24 (m, 2H), 1.04-0.84 (m, 2H), 0.75-0.55 (m, 2H).

UPLC-MS (basic method, 2 min): Rt: 1.02 min, m/z: 400.2 [M+H]⁺.

### N⁴-(5-cyclopentyl-1H-pyrazol-3-yl)-N²-(piperidin-4-yl)pyrimidine-2,4-diamine

To a solution of tert-butyl 4-((4-((5-cyclopropyl-1H-pyrazol-3-yl)amino)pyrimidin-2-yl)amino)piperidine-1-carboxylate (1.02 g, 2.55 mmol) in CH₂Cl₂ (10 mL) was added trifluoroacetic acid (10 mL, 96 mmol) at 0° C. and the reaction mixture was stirred for 1 hour, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The residue was purified by reversed-phase basic prepHPLC, to afford the title compound as a solid (420 mg, 1.41 mmol, 55%).

¹H NMR (400 MHz, methanol-d₄) δ 7.79 (d, J=6.1 Hz, 1H), 6.23-5.95 (m, 2H), 3.95-3.74 (m, 1H), 3.10 (d, J=11.9 Hz, 2H), 2.81-2.66 (m, 2H), 2.03 (d, J=12.4 Hz, 2H), 1.97-1.82 (m, 1H), 1.56-1.38 (m, 2H), 1.05-0.90 (m, 2H), 0.78-0.68 (m, 2H).

UPLC-MS (basic method, 4 min): Rt: 0.97 min, m/z: 300.1 [M+H]⁺.

### Example 7. Synthesis of Compound 30

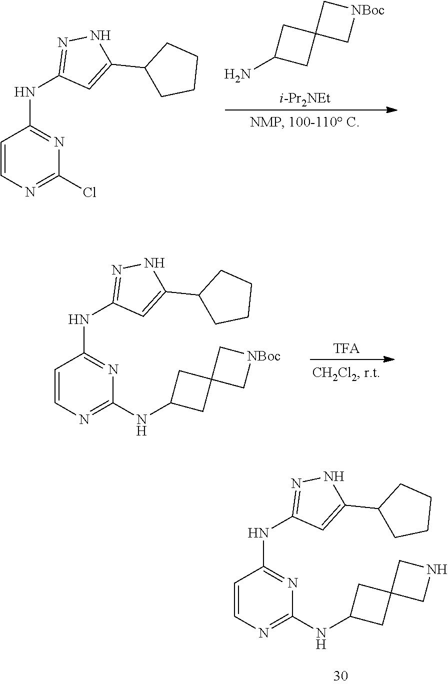 [SMILES: "i-Pr2NEt" ; Clc1nccc(Nc2cc(C3CCCC3)[nH]n2)n1 ; CC(C)(C)OC(=O)N1CC2(CC(N)C2)C1 ; CC(C)(C)OC(=O)N1CC2(CC(Nc3nccc(Nc4cc(C5CCCC5)[nH]n4)n3)C2)C1 ; c1cc(Nc2cc(C3CCCC3)[nH]n2)nc(NC2CC3(CNC3)C2)n1]

### tert-Butyl 6-((4-((5-Cyclopentyl-1H-pyrazol-3-yl)amino)pyrimidin-2-yl)amino)-2-azaspiro[3.3]heptane-2-carboxylate

A solution of 2-chloro-N-(5-cyclopentyl-1H-pyrazol-3-yl)pyrimidin-4-amine (400 mg, 1.52 mmol), 2-Boc-6-amino-2-aza-spiro[3.3]heptane (805 mg, 3.79 mmol) and N,N-diisopropylethylamine (0.925 mL, 5.31 mmol) in n-butanol (15 mL) was heated to 100° C. and stirred for 72 hours. The reaction mixture was then heated to 110° C. and stirred for a further 24 hours, then the reaction temperature was decreased to 100° C. and stirred for a further 92 hours. The reaction mixture was then cooled to ambient temperature, then concentrated to dryness under reduced pressure. The crude was then diluted with water (30 mL), then extracted with CH₂Cl₂ (3×30 mL). The combined organic extracts were washed with a saturated aqueous brine solution (30 mL), dried over Na₂SO₄ then concentrated to dryness under reduced pressure. The residue was purified by normal-phase column chromatography, over silica gel, eluting with a gradient of CH₂Cl₂ to 1:9 2 M NH₃ in MeOH:CH₂Cl₂ to afford the title compound as a solid (376 mg, 0.855 mmol, 56%).

¹H NMR (400 MHz, methanol-d₄) δ 7.84-7.75 (m, 1H), 6.48-6.05 (m, 2H), 4.07-3.77 (m, 5H), 3.18-3.02 (m, 1H), 2.71-2.61 (m, 1H), 2.53-2.44 (m, 1H), 2.24-2.05 (m, 2H), 2.01-1.92 (m, 1H), 1.90-1.80 (m, 1H), 1.78-1.63 (m, 2H), 1.59-1.49 (m, 1H), 1.48-1.27 (m, 11H), 0.96 (t, J=7.3 Hz, 1H)

UPLC-MS (basic method, 4 min): Rt: 1.81 min, m/z: 440.4 [M+H]⁺.

### N⁴-(5-cyclopentyl-1H-pyrazol-3-yl)-N²-(2-azaspiro[3.3]heptan-6-yl)pyrimidine-2,4-diamine

To a solution of tert-butyl 6-((4-((5-cyclopentyl-1H-pyrazol-3-yl)amino)pyrimidin-2-yl)amino)-2-azaspiro[3.3]heptane-2-carboxylate (374 mg, 0.851 mmol) in CH₂Cl₂ (5 mL) was added trifluoroacetic acid (5.0 mL, 65 mmol) at 0° C. The reaction mixture was warmed to ambient temperature and stirred for 30 minutes, after which the volatiles were removed under reduced pressure. The crude residue was loaded onto an SCX cartridge, the cartridge was then washed with methanol, then the compound was eluted with 2 M NH₃ in methanol, and the eluent was concentrated to dryness under reduced pressure. The residue was purified by reversed-phase basic prepHPLC to afford the title compound as a solid (127 mg, 0.367 mmol, 43%).

¹H NMR (400 MHz, DMSO-d₆) δ 9.03 (s, 1H), 7.79 (d, J=5.7 Hz, 1H), 6.47 (s, 1H), 6.18 (d, J=5.8 Hz, 2H), 4.21 (m, 1H), 3.57 (s, 2H), 3.45 (s, 2H), 2.49-2.45 (m, 2H), 2.13-1.95 (m, 4H), 1.83-1.55 (m, 7H).

¹H NMR (400 MHz, methanol-d₄) δ 7.78 (d, J=6.0 Hz, 1H), 6.13 (s, 1H), 4.24 (m, 1H), 3.83 (s, 2H), 3.71 (s, 2H), 3.07 (s, 1H), 2.76-2.54 (m, 2H), 2.29-2.05 (m, 5H), 1.89-1.62 (m, 8H).

UPLC-MS (basic method, 4 min): Rt: 1.17 min, m/z: 340.3 [M+H]⁺.

### Example 8. Synthesis of Compound 31

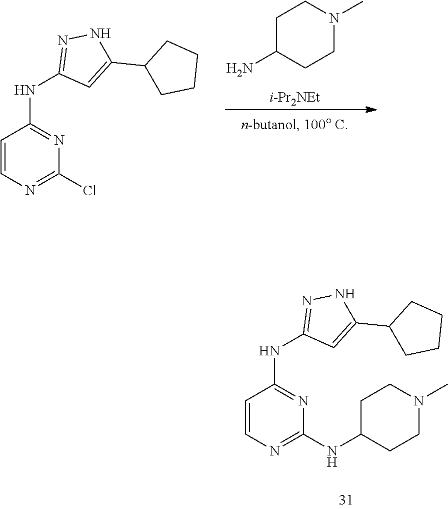

### tert-Butyl (2-(4-((5-Cyclopentyl-1H-pyrazol-3-yl)amino)pyrimidin-2-yl)-2-azaspiro[3.3]heptan-6-yl)carbamate

A solution of 2-chloro-N-(5-cyclopentyl-1H-pyrazol-3-yl)pyrimidin-4-amine (120 mg, 0.455 mmol), 1-methylpiperidin-4-amine (0.143 mL, 1.14 mmol), and N,N-diisopropylethylamine (0.277 mL, 1.59 mmol) in n-butanol (5 mL) was heated to 100° C. and stirred for 116 hours. The reaction mixture was cooled to ambient temperature, then concentrated to dryness under reduced pressure. The residue was then loaded on to an SCX cartridge, the cartridge was washed with methanol. The compound was eluted from the SCX cartridge with 2 M NH₃ in methanol, then the eluent was concentrated to dryness under reduced pressure. The crude residue was purified by reversed-phase acidic prepHPLC, using TFA as the additive, followed by reversed-phase basic prepHPLC, using ammonia as the additive, to afford the title compound as a solid (21 mg, 59 μmol, 13%).

¹H NMR (400 MHz, DMSO-d₆) δ 11.87 (s, 1H), 9.35 (s, 1H), 7.76 (d, J=5.7 Hz, 1H), 6.47 (s, 1H), 6.07 (s, 1H), 3.71-3.64 (m, 1H), 2.99 (s, 1H), 2.75 (d, J=11.1 Hz, 2H), 2.15 (s, 3H), 2.07-1.31 (m, 15H).

¹H NMR (400 MHz, methanol-d₄) δ 7.78 (s, 1H), 6.36 (s, 1H), 6.12 (s, 1H), 3.80 (s, 1H), 3.11-3.05 (m, 1H), 2.89 (d, J=11.7 Hz, 2H), 2.30 (s, 3H), 2.21 (t, J=11.5 Hz, 2H), 2.14-1.99 (m, 4H), 1.87-1.55 (m, 8H).

UPLC-MS (basic method, 4 min): Rt: 1.37 min, m/z: 342.1 [M+H]⁺.

### Example 9. Synthesis of Compound 32

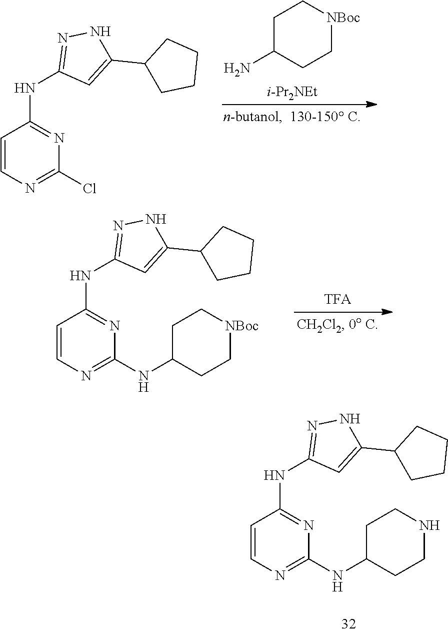 [SMILES: "i-Pr2NEt" ; Clc1nccc(Nc2cc(C3CCCC3)[nH]n2)n1 ; CC(C)(C)OC(=O)N1CCC(N)CC1 ; CC(C)(C)OC(=O)N1CCC(Nc2nccc(Nc3cc(C4CCCC4)[nH]n3)n2)CC1 ; c1cc(Nc2cc(C3CCCC3)[nH]n2)nc(NC2CCNCC2)n1]

### tert-Butyl (2-(4-((5-Cyclopentyl-1H-pyrazol-3-yl)amino)pyrimidin-2-yl)-2-azaspiro[3.3]heptan-6-yl)carbamate

A solution of 2-chloro-N-(5-cyclopentyl-1H-pyrazol-3-yl)pyrimidin-4-amine (120 mg, 0.455 mmol), 4-amino-1-Boc-piperidine (228 mg, 1.14 mmol), and N,N-diisopropylethylamine (0.277 mL, 1.59 mmol) in n-butanol (5 mL) was heated to 130° C. in a microwave reactor and stirred for 2 hours. The reaction mixture was then removed from the microwave reaction, heated to reflux and stirred for 16 hours. The reaction mixture was then heated to 150° C. in a microwave reactor and stirred for 4 hours. The reaction mixture was then cooled to ambient temperature, concentrated to dryness under reduced pressure. The crude was then diluted with water (30 mL) then extracted with CH₂Cl₂ (3×30 mL). The combined organic extracts were washed with a saturated aqueous brine solution (30 mL), dried over Na₂SO₄ then concentrated to dryness under reduced pressure. The residue was purified by normal-phase column chromatography, over silica gel, eluting with a gradient of CH₂Cl₂ to 1:9 2 M NH₃ in MeOH:CH₂Cl₂ to afford the title compound as an oil (125 mg, 0.292 mmol, 64%).

¹H NMR (400 MHz, DMSO-d₆) δ 11.87 (s, 1H), 9.27 (s, 1H), 7.77 (s, 1H), 6.31 (s, 1H), 6.16 (s, 1H) 4.04-3.69 (m, 3H), 3.08-2.72 (m, 4H), 2.05-1.81 (m, 3H), 1.75-1.56 (m, 5H), 1.46-1.28 (m, 11H), 1.11-0.99 (m, 1H).

UPLC-MS (basic method, 4 min): Rt: 1.11 min, m/z: 428.3 [M+H]⁺.

### N⁴-(5-cyclopentyl-1H-pyrazol-3-yl)-N²-(piperidin-4-yl)pyrimidine-2,4-diamine

To a solution of tert-butyl (2-(4-((5-cyclopentyl-1H-pyrazol-3-yl)amino)pyrimidin-2-yl)-2-azaspiro[3.3]heptan-6-yl)carbamate (107 mg, 0.250 mmol) in CH₂Cl₂ (1.5 mL) was added trifluoroacetic acid (1.5 mL, 20 mmol) at 0° C. and the reaction mixture was stirred for 30 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The crude residue was purified by reversed-phase acidic prepHPLC, using TFA as the additive, followed by reversed-phase basic prepHPLC, using ammonia as the additive, to afford the title compound as a solid (48 mg, 0.15 mmol, 59%).

¹H NMR (400 MHz, DMSO-d₆) δ 11.86 (s, 1H), 9.35 (s, 1H), 7.76 (d, J=5.7 Hz, 1H), 6.42 (m, 2H), 6.08 (s, 1H), 3.71 (m, 1H), 3.06-2.89 (m, 3H), 1.99 (s, 2H), 1.81 (dd, J=12.7, 3.6 Hz, 2H), 1.74-1.54 (m, 7H), 1.38-1.22 (m, 3H).

¹H NMR (400 MHz, methanol-d₄) δ 7.79 (d, J=6.0 Hz, 1H), 6.31 (s, 1H), 6.12 (s, 1H), 3.89 (s, 1H), 3.22-3.01 (m, 3H), 2.85-2.66 (m, 2H), 2.20-1.97 (m, 4H), 1.93-1.62 (m, 7H), 1.48 (qd, J=11.5, 4.0 Hz, 2H).

UPLC-MS (basic method, 4 min): Rt: 1.20 min, m/z: 328.1 [M+H]⁺.

### Example 10. Synthesis of Compound 33

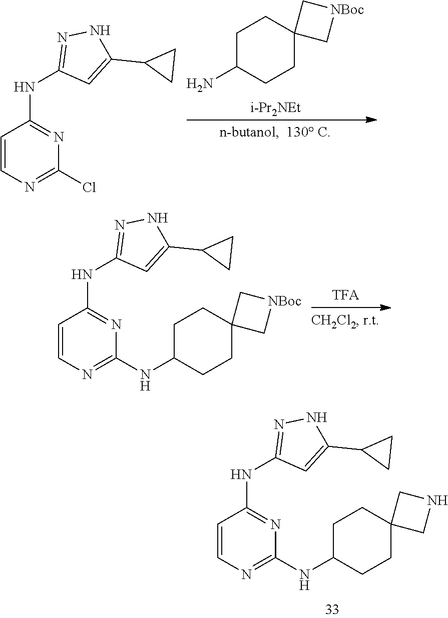

### tert-Butyl 7-((4-((5-Cyclopropyl-1H-pyrazol-3-yl)amino)pyrimidin-2-yl)amino)-2-azaspiro[3.5]nonane-2-carboxylate

A solution of 2-chloro-N-(5-cyclopropyl-1H-pyrazol-3-yl)pyrimidin-4-amine (150 mg, 0.636 mmol), tert-butyl N-[(4-amino-1-methyl-cyclohexyl)methyl]-N-methyl-carbamate (245 mg, 0.996 mmol) and N,N-diisopropylethylamine (0.388 mL, 2.23 mmol) in n-butanol (4 mL) was heated to 130° C. in a sealed tube and stirred for 72 hours. The reaction mixture was then cooled to ambient temperature, and the reaction mixture was concentrated to dryness under reduced pressure. The crude was then diluted with water (50 mL), then extracted with EtOAc (3×50 mL). The combined organic extracts were dried over Na₂SO₄ then concentrated to dryness under reduced pressure. The residue was purified by normal-phase column chromatography, over silica gel, eluting with a gradient of CH₂Cl₂ to 1:9 2 M NH₃ in MeOH:CH₂Cl₂ to afford the title compound as a solid (245 mg, 0.557 mmol, 88%)

¹H NMR (400 MHz, DMSO-d₆) δ 11.69 (s, 1H), 8.82 (s, 1H), 7.79 (d, J=4.4 Hz, 1H), 6.16 (s, 1H), 5.90 (s, 1H), 3.73-3.64 (m, 1H), 3.57 (s, 2H), 3.51 (s, 2H), 1.92-1.80 (m, 5H), 1.59-1.47 (m, 2H), 1.40 (s, 9H), 1.37-1.28 (m, 2H), 0.94-0.82 (m, 2H), 0.71-0.63 (m, 2H)

UPLC-MS (basic method, 2 min): Rt: 1.08 min, m/z: 440.6 [M+H]⁺.

### N⁴-(5-Cyclopropyl-1H-pyrazol-3-yl)-N²-(2-azaspiro[3.5]nonan-7-yl)pyrimidine-2,4-diamine

To a solution of tert-butyl 7-((4-((5-cyclopropyl-1H-pyrazol-3-yl)amino)pyrimidin-2-yl)amino)-2-azaspiro[3.5]nonane-2-carboxylate (80 mg, 0.18 mmol) in CH₂Cl₂ (3 mL) was added trifluoroacetic acid (3.0 mL, 39 mmol) at 0° C. The reaction mixture was warmed to ambient temperature and stirred for 30 minutes, after which the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure. The residue was purified by reversed-phase basic prepHPLC to afford the title compound as a solid (39 mg, 0.11 mmol, 63%).

¹H NMR (400 MHz, DMSO-d₆) δ 11.77 (s, 1H), 9.36 (s, 1H), 7.76 (s, 1H), 6.58-5.83 (m, 3H), 3.72-3.58 (m, 1H), 3.53 (s, 2H), 3.47 (s, 2H), 2.04-1.72 (m, 4H), 1.56-1.36 (m, 2H), 1.31-1.17 (m, 3H), 0.92-0.86 (m, 2H), 0.68-0.61 (m, 2H).

¹H NMR (400 MHz, methanol-d₄) δ 7.78 (d, J=6.1 Hz, 1H), 6.26-5.93 (m, 2H), 3.76-3.67 (m, 1H), 3.65 (s, 2H), 3.57 (s, 2H), 2.13-2.04 (m, 2H), 2.02-1.93 (m, 2H), 1.93-1.85 (m, 1H), 1.70-1.59 (m, 2H), 1.39-1.23 (m, 2H), 1.01-0.89 (m, 2H), 0.76-0.65 (m, 2H)

UPLC-MS (basic method, 4 min): Rt: 0.99 min, m/z: 340.2 [M+H]⁺.

### Example 11. Synthesis of Compound 34

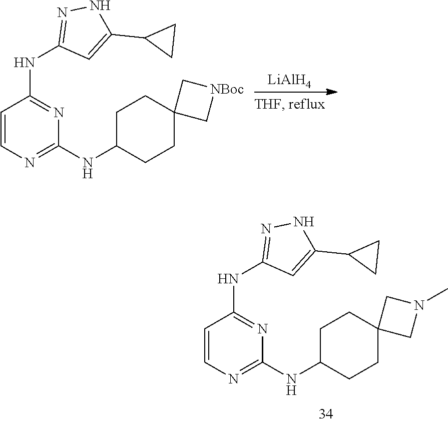 [SMILES: CC(C)(C)OC(=O)N1CC2(CCC(Nc3nccc(Nc4cc(C5CC5)[nH]n4)n3)CC2)C1 ; "LiAlH4" ; CN1CC2(CCC(Nc3nccc(Nc4cc(C5CC5)[nH]n4)n3)CC2)C1]

### N⁴-(5-cyclopropyl-1H-pyrazol-3-yl)-N²-(2-methyl-2-azaspiro[3.5]nonan-7-yl)pyrimidine-2,4-diamine

To a solution of tert-butyl 7-((4-((5-cyclopropyl-1H-pyrazol-3-yl)amino)pyrimidin-2-yl)amino)-2-azaspiro[3.5]nonane-2-carboxylate (140 mg, 0.319 mmol) in anhydrous THF (20 mL) was added a 2.4 M solution of lithium aluminum hydride in THF (1.3 mL, 3.2 mmol) dropwise at 0° C. The reaction mixture was heated to reflux and stirred for 2 hours. The reaction mixture was then cooled to 0° C., then quenched with water (130 μL), then a 15% w/v sodium hydroxide solution (130 μL), followed by water (390 μL). The reaction mixture was warmed to ambient temperature and stirred for 15 minutes, after which magnesium sulfate (ca. 1 g) was added and the reaction mixture was stirred for a further 15 minutes. The reaction mixture was passed through a pad of celite, and the filter cake was washed with EtOAc (10 mL). The eluent was then concentrated to dryness under reduced pressure. The residue was purified by reversed-phase basic prepHPLC to afford the title compound as a solid (54 mg, 0.15 mmol, 48%).

¹H NMR (400 MHz, DMSO-d₆, 90° C.) δ 11.69 (s, 1H), 8.86 (s, 1H), 7.79 (d, J=5.7 Hz, 1H), 6.47-5.30 (m, 3H), 3.68 (dddd, J=14.2, 10.2, 7.7, 3.8 Hz, 1H), 2.94 (s, 2H), 2.87 (s, 2H), 2.24 (s, 3H), 1.93-1.77 (m, 5H), 1.47 (td, J=12.6, 3.3 Hz, 2H), 1.28 (tdd, J=12.8, 10.2, 3.3 Hz, 2H), 0.92-0.84 (m, 2H), 0.71-0.63 (m, 2H).

¹H NMR (400 MHz, methanol-d₄) δ 7.77 (d, J=5.9 Hz, 1H), 6.33-5.88 (m, 2H), 3.75-3.61 (m, 1H), 3.15 (s, 2H), 3.07 (s, 2H), 2.37 (s, 3H), 2.04-1.83 (m, 5H), 1.66-1.51 (m, 2H), 1.39-1.23 (m, 2H), 1.05-0.89 (m, 2H), 0.79-0.63 (m, 2H).

UPLC-MS (basic method, 4 min): Rt: 1.19 min, m/z: 354.1 [M+H]⁺.

### Example 12. Synthesis of Compound 35

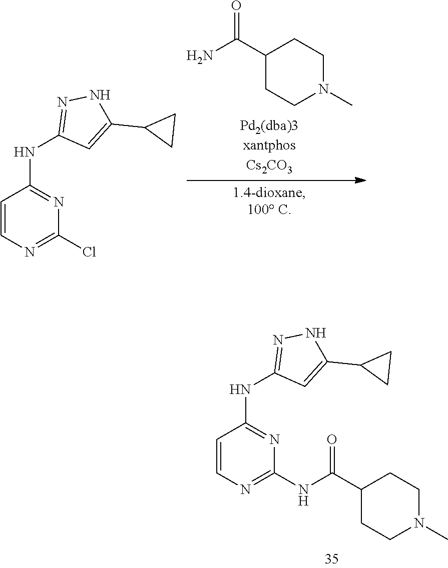

### N-(4-((5-cyclopropyl-1H-pyrazol-3-yl)amino)pyrimidin-2-yl)-1-methylpiperidine-4-carboxamide

A suspension of 2-chloro-N-(5-cyclopropyl-1H-pyrazol-3-yl)pyrimidin-4-amine (250 mg, 1.06 mmol), 1-methylpiperidine-4-carboxamide (186 mg, 1.30 mmol), cesium carbonate (1.04 g, 3.18 mmol), tris(dibenzylideneacetone)dipalladium(0) (48 mg, 53 μmol) and 4,5-bis(diphenylphosphino)-9,9-dimethylxanthene (92 mg, 0.16 mmol) in 1,4-dioxane (10 mL) was heated to 100° C. in a microwave reactor and stirred for 16 hours. The reaction mixture was then cooled to ambient temperature, and filtered through a pad of celite. The eluent was concentrated to dryness under reduced pressure. The residue was then loaded on to an SCX cartridge, the cartridge was washed with methanol. The compound was eluted from the SCX cartridge with 2 M NH₃ in methanol, then the eluent was concentrated to dryness under reduced pressure. The residue was purified by normal-phase column chromatography, over silica gel, eluting with a gradient of CH₂Cl₂ to 3:17 2 M NH₃ in MeOH:CH₂Cl₂The crude residue was purified by reversed-phase acidic prepHPLC, using TFA as the additive, followed by reversed-phase basic prepHPLC, using ammonia as the additive, to afford the title compound as a solid (7 mg, 2%).

¹H NMR (400 MHz, methanol-d₄) δ 8.11 (s, 1H), 6.41 (s, 1H), 5.49 (s, 1H), 3.00-2.92 (m, 2H), 2.56-2.39 (m, 1H), 2.29 (s, 3H), 2.17-2.07 (m, 2H), 1.98-1.78 (m, 5H), 1.00-0.84 (m, 2H), 0.81-0.61 (m, 2H).

UPLC-MS (basic method, 2 min): Rt: 1.09 min, m/z: 342.3 [M+H]⁺.

### Example 13. Synthesis of Compound 36

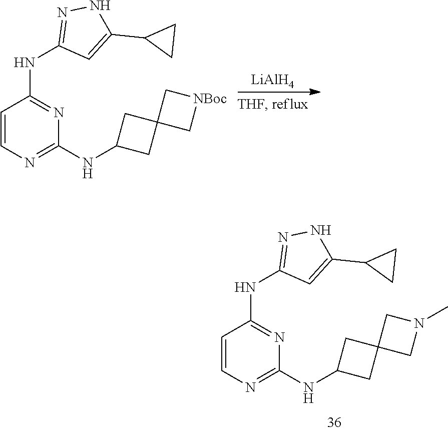 [SMILES: CC(C)(C)OC(=O)N1CC2(CC(Nc3nccc(Nc4cc(C5CC5)[nH]n4)n3)C2)C1 ; "LiAlH4" ; CN1CC2(CC(Nc3nccc(Nc4cc(C5CC5)[nH]n4)n3)C2)C1]

### N⁴-(5-cyclopropyl-1H-pyrazol-3-yl)-N²-(2-methyl-2-azaspiro[3.3]heptan-6-yl)pyrimidine-2,4-diamine

To a solution of tert-butyl 6-((4-((5-cyclopropyl-1H-pyrazol-3-yl)amino)pyrimidin-2-yl)amino)-2-azaspiro[3.3]heptane-2-carboxylate (2.30 g, 5.59 mmol) in anhydrous THF (100 mL) was added a 2.4 M solution of lithium aluminum hydride in THF (23.3 mL, 55.9 mmol) dropwise at 0° C. The reaction mixture was heated to reflux and stirred for 1.5 hours. The reaction mixture was then cooled to 0° C., then quenched with water (2.1 mL), then a 15% w/v NaOH solution (2.1 mL), followed by water (6.3 mL). The reaction mixture was warmed to ambient temperature and stirred for 15 minutes, after which magnesium sulfate (ca. 20 g) was added and the reaction mixture was stirred for a further 15 minutes. The reaction mixture was passed through a pad of celite, and the filter cake was washed with EtOAc (10 mL). The eluent was then concentrated to dryness under reduced pressure. 100 mg of the crude residue was purified by reversed-phase basic prepHPLC to afford the title compound as a solid (66 mg, 0.20 mmol, 3.6%).

¹H NMR (400 MHz, DMSO-d₆) δ 11.91 (s, 1H), 9.30 (s, 1H), 7.75 (d, J=5.7 Hz, 1H), 6.83 (s, 1H), 6.41-5.91 (m, 2H), 4.17 (h, J=8.0 Hz, 1H), 3.15 (s, 2H), 3.02 (s, 2H), 2.43-2.33 (m, 2H), 2.15 (s, 3H), 2.04-1.95 (m, 2H), 1.84 (tt, J=8.9, 5.2 Hz, 1H), 0.98-0.82 (m, 2H), 0.71-0.59 (m, 2H).

¹H NMR (400 MHz, methanol-d₄) δ 7.77 (d, J=5.9 Hz, 1H), 6.34-5.88 (m, 2H), 4.29-4.13 (m, 1H), 3.38 (s, 2H), 3.24 (s, 2H), 2.65-2.53 (m, 2H), 2.31 (s, 3H), 2.13-2.00 (m, 2H), 1.94-1.84 (m, 1H), 1.00-0.94 (m, 2H), 0.75-0.71 (m, 2H).

UPLC-MS (basic method, 4 min): Rt: 1.09 min, m/z: 326.3 [M+H]⁺.

### Example 14. Synthesis of Compound 37

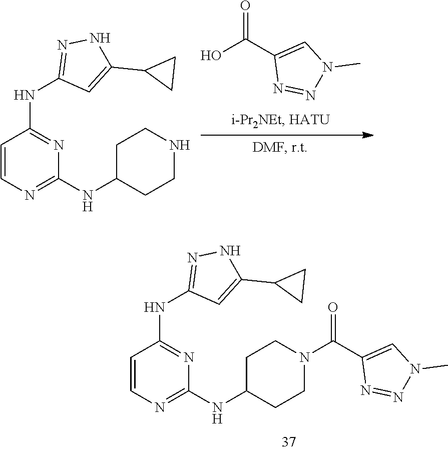

### (4-((4-((5-Cyclopropyl-1H-pyrazol-3-yl)amino)pyrimidin-2-yl)amino)piperidin-1-yl)(1-methyl-1H-1,2,3-triazol-4-yl)methanone

To a solution of 1-methyl-1H-1,2,3-triazole-4-carboxylic acid (44 mg, 0.34 mmol), and N,N-diisopropylethylamine (65 μL, 0.37 mmol) in DMF (1 mL) was added HATU (130 mg, 0.343 mmol) and the reaction mixture was stirred at ambient temperature for one hour. A solution of N⁴-(5-cyclopentyl-1H-pyrazol-3-yl)-N²-(2-azaspiro[3.3]heptan-6-yl)pyrimidine-2,4-diamine (28 mg, 93 μmol) in DMF (2 mL) was added and the reaction mixture was stirred at ambient temperature for a further 2 hours. The reaction mixture was loaded onto an SCX cartridge, the cartridge was then washed with methanol, then the compound was eluted with 2 M NH₃ in methanol, and the eluent was concentrated to dryness under reduced pressure. The residue was purified successively by reversed-phase basic prepHPLC, to afford the title compound as a solid (28 mg, 64 mol, 68%).

¹H NMR (400 MHz, DMSO-d₆) δ 11.89 (s, 1H), 9.27 (s, 1H), 8.46 (s, 1H), 7.79 (s, 1H), 6.61 (s, 1H), 6.36-6.06 (m, 2H), 4.85-4.66 (m, 1H), 4.53-4.29 (m, 1H), 4.08 (s, 3H), 4.04-3.99 (m, 2H), 3.03-2.70 (m, 1H), 2.05-1.90 (m, 2H), 1.88-1.76 (m, 1H), 1.54-1.37 (m, 2H), 1.00-0.80 (m, 2H), 0.75-0.53 (m, 2H).

UPLC-MS (basic method, 4 min): Rt: 1.41 min, m/z: 409.3 [M+H]⁺.

### Example 15. Synthesis of Compound 38

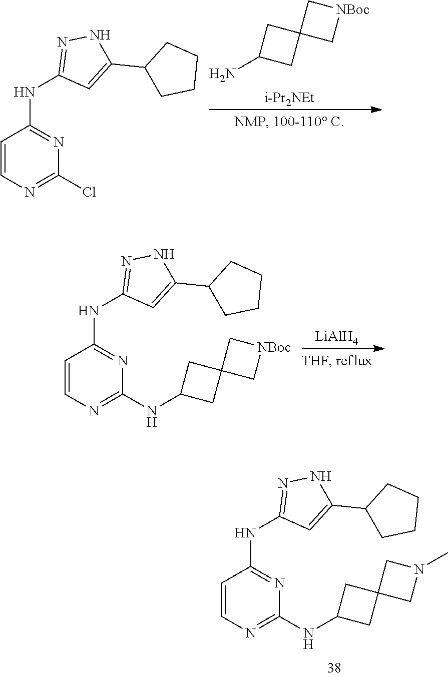

### N⁴-(5-cyclopentyl-1H-pyrazol-3-yl)-N²-(2-methyl-2-azaspiro[3.3]heptan-6-yl)pyrimidine-2,4-diamine

To a solution of tert-butyl 6-((4-((5-cyclopentyl-1H-pyrazol-3-yl)amino)pyrimidin-2-yl)amino)-2-azaspiro[3.3]heptane-2-carboxylate (83 mg, 0.19 mmol) in anhydrous THF (10 mL) was added a 2.4 M solution of lithium aluminum hydride in THF (0.79 mL, 1.89 mmol) dropwise at 0° C. The reaction mixture was heated to reflux and stirred for 2 hours. The reaction mixture was then cooled to 0° C., then quenched with water (72 μL), then a 15% w/v sodium hydroxide solution (72 μL), followed by water (215 μL). The reaction mixture was warmed to ambient temperature and stirred for 15 minutes, after which magnesium sulfate (ca. 1 g) was added and the reaction mixture was stirred for a further 15 minutes. The reaction mixture was passed through a pad of celite, and the filter cake was washed with EtOAc (20 mL). The eluent was then concentrated to dryness under reduced pressure. The residue was purified by reversed-phase basic prepHPLC to afford the title compound as a solid (13 mg, 33 mol, 18%).

¹H NMR (400 MHz, methanol-d₄) δ 7.77 (s, 1H), 6.39 (s, 1H), 6.13 (s, 1H), 4.25 (d, J=12.4 Hz, 1H), 3.39 (s, 2H), 3.27 (s, 2H), 3.18-2.96 (m, 1H), 2.68-2.53 (m, 2H), 2.32 (s, 3H), 2.09 (m, 4H), 1.92-1.59 (m, 6H).

UPLC-MS (basic method, 4 min): Rt: 1.13 min, m/z: 352.2 [M+H]⁺.

### Example 16. Synthesis of Compound 39

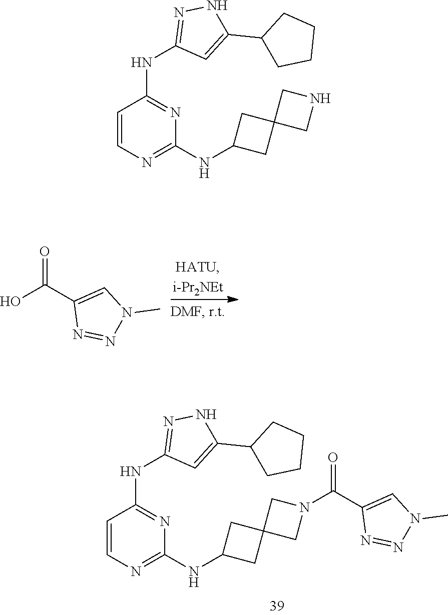

### (6-((4-((5-Cyclopentyl-1H-pyrazol-3-yl)amino)pyrimidin-2-yl)amino)-2-azaspiro[3.3]heptan-2-yl)(1-methyl-1H-1,2,3-triazol-4-yl)methanone

To a solution of 1-methyl-1H-1,2,3-triazole-4-carboxylic acid (44 mg, 0.34 mmol), and N,N-diisopropylethylamine (65 μL, 0.37 mmol) DMF (2 mL) was added HATU (130 mg, 0.343 mmol) and the reaction mixture was stirred at ambient temperature for one hour. A solution of N⁴-(5-cyclopentyl-1H-pyrazol-3-yl)-N²-(2-azaspiro[3.3]heptan-6-yl)pyrimidine-2,4-diamine (97 mg, 0.29 mmol) in DMF (3 mL) was added and the reaction mixture was stirred at ambient temperature for a further 20 hours. The reaction mixture was loaded onto an SCX cartridge, the cartridge was then washed with methanol, then the compound was eluted with 2 M NH₃ in methanol, and the eluent was concentrated to dryness under reduced pressure. The residue was purified successively by reversed-phase acidic prepHPLC, using TFA as a modified, then by reversed-phase basic prepHPLC, using ammonia as the modifier, to afford the title compound as a solid (29 mg, 62 mol, 22%).

¹H NMR (400 MHz, DMSO-d₆) δ 11.88 (s, 1H), 9.30 (s, 1H), 8.50 (s, 1H), 6.87 (s, 1H), 6.41 (s, 1H), 6.18 (s, 1H), 4.61 (s, 1H), 4.47 (s, 1H), 4.25 (d, J=7.9 Hz, 1H), 4.10 (s, 1H), 4.07 (d, J=2.4 Hz, 3H), 3.99 (s, 1H), 2.99 (q, J=8.2 Hz, 1H), 2.57 (s, 2H), 2.19 (s, 2H), 2.01 (s, 2H), 1.78-1.47 (m, 6H).

UPLC-MS (basic method, 4 min): Rt: 1.30 min, m/z: 449.4 [M+H]⁺.

### Example 17: Synthesis of Compound 20

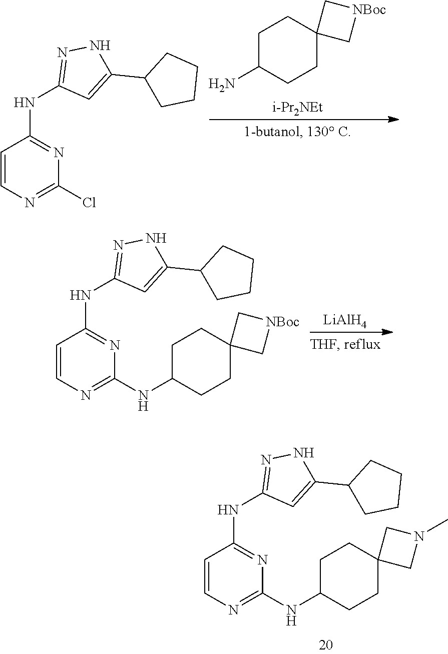

### tert-Butyl 7-[[4-[(5-Cyclopentyl-1H-pyrazol-3-yl)amino]pyrimidin-2-yl]amino]-2-azaspiro[3.5]nonane-2-carboxylate

A solution of 2-chloro-N-(5-cyclopentyl-1H-pyrazol-3-yl)pyrimidin-4-amine (350 mg, 1.33 mmol), tert-butyl 7-amino-2-azaspiro[3.5]nonane-2-carboxylate (478 mg, 1.99 mmol) and N,N-diisopropylethylamine (0.81 mL, 4.6 mmol) in 1-butanol (8 mL) was heated to 130° C. and stirred for 96 hours. The reaction mixture was then cooled to ambient temperature, diluted with water (20 mL) and extracted with CH₂Cl₂ (3×15 mL). The organics were combined and dried over Na₂SO₄ and concentrated to dryness under reduced pressure. The residue was purified by normal-phase column chromatography, over silica gel, eluting with a gradient of CH₂Cl₂ to 9:1 CH₂Cl₂:0.1% v/v triethylamine in MeOH to afford the title compound as a solid (410 mg, 0.877 mmol, 66%).

¹H NMR (400 MHz, chloroform-d) δ 7.89 (d, J=5.8 Hz, 1H), 6.23-6.01 (m, 2H), 3.84-3.70 (m, 1H), 3.60 (s, 2H), 3.56 (s, 2H), 3.01 (p, J=8.1 Hz, 1H), 2.10-2.01 (m, 2H), 2.00-1.91 (m, 2H), 1.90-1.81 (m, 2H), 1.79-1.70 (m, 2H), 1.67-1.50 (m, 6H), 1.43 (s, 9H), 1.28-1.11 (m, 2H).

UPLC-MS (basic method, 2 min): Rt: 1.15 min, m/z=468.3 [M+H]⁺.

### N⁴-(5-Cyclopentyl-1H-pyrazol-3-yl)-N²-(2-methyl-2-azaspiro[3.5]nonan-7-yl)pyrimidine-2,4-diamine

To a solution of tert-butyl 7-[[4-[(5-cyclopentyl-1H-pyrazol-3-yl)amino]pyrimidin-2-yl]amino]-2-azaspiro[3.5]nonane-2-carboxylate (200 mg, 0.428 mmol) in anhydrous THF (8 mL) was added a 2.4 M solution of lithium aluminum hydride in THF (1.78 mL, 4.277 mmol) and the reaction mixture heated to reflux and stirred for 3 hours. The reaction mixture was allowed to cool to room temperature, then quenched with water (1 mL), then a 15% w/v sodium hydroxide solution (1 mL), followed by water (1 mL). The reaction mixture was stirred for 15 minutes, after which magnesium sulfate (ca. 1 g) was added, and the reaction mixture was stirred for a further 15 minutes. The reaction mixture was passed through a pad of celite, and the filter cake was washed with EtOAc (20 mL). The eluent was concentrated to dryness under reduced pressure. The crude compound was loaded onto an SCX-2 cartridge, the cartridge was then washed with methanol, then the compound was eluted with 2 M NH₃ in methanol, and the eluent was concentrated to dryness under reduced pressure to afford the title compound a solid (107 mg, 0.275 mmol, 64%).

¹H NMR (400 MHz, DMSO-d₆) δ 11.86 (s, 1H), 9.27 (s, 1H), 7.76-7.72 (m, 1H), 6.39 (s, 1H), 6.09 (s, 1H), 3.68-3.61 (m, 1H), 3.02-2.94 (m, 1H), 2.91 (s, 2H), 2.83 (s, 2H), 2.21 (s, 3H), 2.01-1.97 (m, 2H), 1.91-1.83 (m, 2H), 1.82-1.75 (m, 2H), 1.62-1.57 (m, 6H), 1.47-1.36 (m, 2H), 1.30-1.16 (m, 2H).

¹H NMR (400 MHz, methanol-d₄) δ 7.76 (s, 1H), 6.35 (s, 1H), 6.09 (s, 1H), 3.71 (s, 1H), 3.15 (s, 1H), 3.07 (s, 2H), 2.38 (s, 1H), 2.10-2.05 (m, 2H), 2.01-1.90 (m, 4H), 1.82-1.76 (m, 2H), 1.70-1.66 (m, 6H), 1.63-1.53 (m, 2H), 1.37-1.27 (m, 2H).

UPLC-MS (basic method, 4 min): Rt: 1.39 min, m/z=382.2 [M+H]⁺.

### Example 18. Synthesis of Compound 40

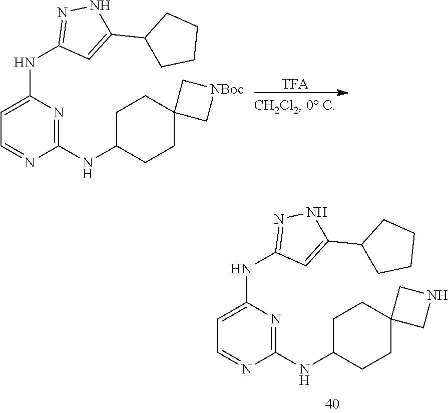 [SMILES: CC(C)(C)OC(=O)N1CC2(CCC(Nc3nccc(Nc4cc(C5CCCC5)[nH]n4)n3)CC2)C1 ; c1cc(Nc2cc(C3CCCC3)[nH]n2)nc(NC2CCC3(CC2)CNC3)n1 ; "TFA"]

### N²-(2-Azaspiro[3.5]nonan-7-yl)-N⁴-(5-cyclopentyl-1H-pyrazol-3-yl)pyrimidine-2,4-diamine

To a solution of tert-butyl 7-[[4-[(5-Cyclopentyl-1H-pyrazol-3-yl)amino]pyrimidin-2-yl]amino]-2-azaspiro[3.5]nonane-2-carboxylate (170 mg, 0.364 mmol) in CH₂Cl₂ (4 mL) was added trifluoroacetic acid (1.0 mL, 13 mmol) at 0° C. for 30 minutes, then the reaction mixture was concentrated to dryness under reduced pressure. The crude residue was loaded onto an SCX cartridge, the cartridge was then washed with methanol, then the compound was eluted with 2 M NH₃ in methanol, and the eluent was concentrated to dryness under reduced pressure to afford the title compound as a solid (62 mg, 0.17 mmol, 46%).

¹H NMR (400 MHz, DMSO-d₆) δ 11.84 (s, 1H), 9.38 (s, 1H), 7.77-7.72 (m, 1H), 6.43 (s, 2H), 6.06 (s, 1H), 3.66-3.61 (m, 1H), 3.42-3.34 (m, 1H), 3.24 (s, 2H), 3.16 (s, 2H), 3.02-2.93 (m, 1H), 2.00-1.92 (m, 4H), 1.82-1.75 (m, 2H), 1.71-1.65 (m, 2H), 1.64-1.57 (m, 4H), 1.41-1.34 (m, 2H), 1.25-1.18 (m, 2H).

¹H NMR (400 MHz, methanol-d₄) δ 7.76 (d, J=6.0 Hz, 1H), 6.22 (s, 1H), 6.08 (s, 1H), 3.73-3.69 (m, 1H), 3.48 (s, 2H), 3.39 (s, 2H), 3.13-2.99 (m, 1H), 2.10-2.01 (m, 4H), 1.98-1.90 (m, 2H), 1.81-1.75 (m, 2H), 1.73-1.56 (m, 4H), 1.60-1.48 (m, 2H), 1.36-1.23 (m, 2H).

UPLC-MS (basic method, 4 min): Rt: 1.23 min, m/z=368.1 [M+H]⁺.

### Example 19. Synthesis of Compound 41

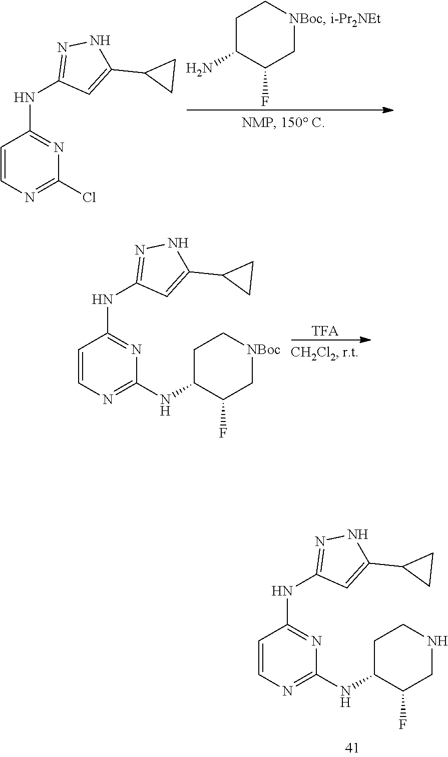

### tert-Butyl (3S,4R)-4-[[4-[(5-Cyclopropyl-1H-pyrazol-3-yl)amino]pyrimidin-2-yl]amino]-3-fluoro-piperidine-1-carboxylate

A solution of 2-chloro-N-(5-cyclopropyl-1H-pyrazol-3-yl)pyrimidin-4-amine (1.747 g, 7.413 mmol), (3S,4R)-tert-butyl 4-amino-3-fluoropiperidine-1-carboxylate (1.78 g, 8.16 mmol) and N,N-diisopropylethylamine (4.3 mL, 24 mmol) in NMP (31 mL) was heated to 150° C. and stirred for 40 hours. The reaction mixture was then cooled to room temperature, then added dropwise to ice water (ca. 450 mL) resulting in the formation of a precipitate. The precipitate was collected by filtration, then dissolved in EtOAc, dried over Na₂SO₄ then concentrated to dryness under reduced pressure. The crude material was purified by normal-phase column chromatography, over silica gel, eluting with a gradient of CH₂Cl₂ to 1:4 MeOH:CH₂Cl₂ to afford the title compound as a solid (560 mg, 1.34 mmol, 16%).

¹H NMR (400 MHz, DMSO-d₆, 110° C.) δ 11.69 (s, 1H), 8.90 (s, 1H), 7.84 (d, J=5.7 Hz, 1H), 6.23 (s, 1H), 5.96 (s, 1H), 4.85 (d, J=49.2 Hz, 1H), 4.26 (t, J=13.0 Hz, 1H), 4.18-4.00 (m, 2H), 3.09 (dd, J=38.4, 14.7 Hz, 1H), 2.91-2.82 (m, 1H), 1.92-1.81 (m, 1H), 1.79-1.70 (m, 2H), 1.43 (s, 9H), 0.94-0.82 (m, 2H), 0.74-0.63 (m, 2H).

¹⁹F NMR (376 MHz, DMSO-d₆) δ −202.3-−203.7 (m).

UPLC-MS (basic method, 4 min): Rt: 1.64 min, m/z=418.3 [M+H]⁺.

### N₄-(5-Cyclopropyl-1H-pyrazol-3-yl)-N₂-[(3S,4R)-3-fluoro-4-piperidyl]pyrimidine-2,4-diamine

To a solution of tert-butyl (3S,4R)-4-[[4-[(5-Cyclopropyl-1H-pyrazol-3-yl)amino]pyrimidin-2-yl]amino]-3-fluoro-pperidine-1-carboxylate (125 mg, 0.299 mmol) in CH₂Cl₂ (1.9 mL) was added trifluoroacetic acid (0.50 mL, 6.5 mmol) then the reaction mixture was stirred at room temperature for 16 hours. The reaction mixture was then concentrated to dryness under reduced pressure. The residue was then loaded onto a SCX-2 cartridge. The cartridge was washed with MeOH (25 mL) then the compound was eluted with 2 M NH₃ in MeOH (25 mL). The eluent was concentrated to dryness under reduced pressure to afford the title compound as a solid (79 mg, 0.24 mmol, 80%).

¹H NMR (400 MHz, DMSO-d₆) δ 11.91 (s, 1H), 9.35 (s, 1H), 7.81 (s, 1H), 6.45 (s, 1H), 6.14 (s, 1H), 4.76 (d, J=50.6 Hz, 1H), 4.09-3.82 (m, 1H), 3.13 (t, J=12.6 Hz, 1H), 2.96 (d, J=13.0 Hz, 1H), 2.78-2.52 (m, 2H), 1.88-1.78 (m, 1H), 1.71-1.56 (m, 2H), 1.01-0.76 (m, 2H), 0.73-0.57 (m, 2H).

¹⁹F NMR (376 MHz, DMSO-d₆) (376 MHz, DMSO-d₆) δ −201.2-−204.0 (m)

UPLC-MS (basic method, 4 min): Rt: 1.03 min, m/z=318.3 [M+H]⁺

### Example 20. Synthesis of Compound 42

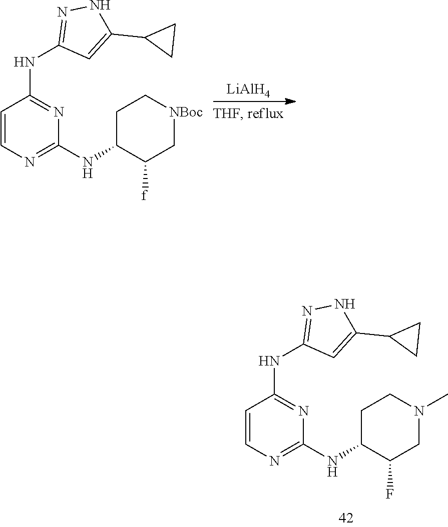

### N₄-(5-Cyclopropyl-1H-pyrazol-3-yl)-N₂-[(3S,4R)-3-fluoro-1-methyl-4-piperidyl]pyrimidine-2,4-diamine

To a solution of tert-butyl (3S,4R)-4-[[4-[(5-cyclopropyl-1H-pyrazol-3-yl)amino]pyrimidin-2-yl]amino]-3-fluoro-piperidine-1-carboxylate (125 mg, 0.299 mmol) in anhydrous THF (6 mL) was added a 2.4M solution of lithium aluminum hydride in THF (1.2 mL, 3.0 mmol) at 0° C. The reaction mixture was heated to reflux and stirred for 4 hours. The reaction mixture was cooled to 0° C., then quenched with water (0.75 mL), then a 10% w/v aqueous NaOH solution (0.75 mL) then water (1.5 mL). MgSO₄ (Ca. 1 g) was added then the reaction mixture was warmed to room temperature and stirred for a further 10 minutes. The reaction mixture was filtered through celite, and the filter cake was washed with EtOAc (20 mL). The filtrate was concentrated to dryness under reduced pressure. The residue was purified by normal-phase column chromatography eluting with a gradient of CH₂Cl₂ to 1:9 MeOH:CH₂Cl₂ to afford the title compound as a solid (15 mg, 44 μmol, 15%).

¹H NMR (400 MHz, DMSO-d₆) δ 11.78 (s, 1H), 9.01 (s, 1H), 7.83 (d, J=5.7 Hz, 1H), 6.20 (s, 1H), 5.97 (s, 1H), 4.80 (d, J=49.5 Hz, 1H), 4.10-3.80 (m, 1H), 3.16-3.02 (m, 2H), 2.85-2.78 (m, 1H), 2.34-2.08 (m, 5H), 1.94-1.80 (m, 2H), 1.77-1.65 (m, 1H), 0.93-0.80 (m, 2H), 0.73-0.61 (m, 2H).

¹⁹F NMR (376 MHz, DMSO-d₆) δ −197.76-−200.33 (m).

UPLC-MS (basic method, 4 min): Rt: 1.14 min, m/z=323.3 [M+H]⁺.

### Example 21. Synthesis of Compound 43

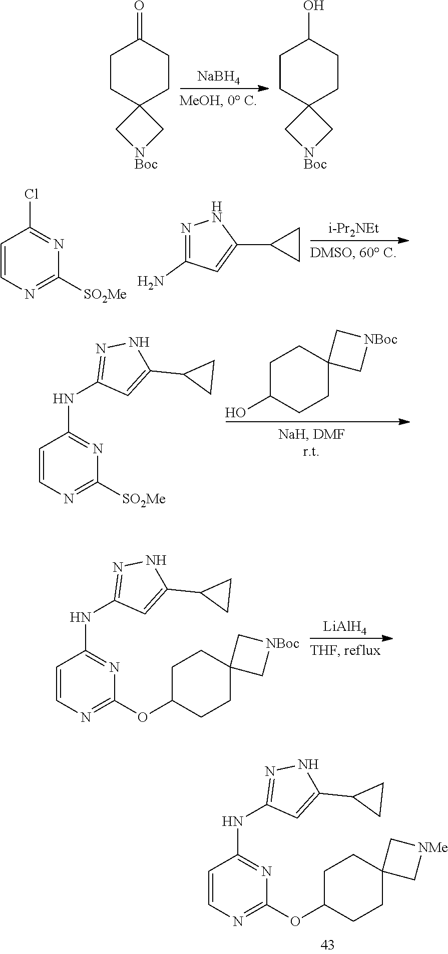

### tert-Butyl 7-hydroxy-2-azaspiro[3.5]nonane-2-carboxylate

To a solution of tert-butyl 7-oxo-2-azaspiro[3.5]nonane-2-carboxylate (1.40 g, 5.85 mmol) in methanol (4 mL) was added sodium borohydride (266 mg, 7.02 mmol) in a portion wise manner at 0° C. The reaction mixture was warmed to room temperature and stirred for 30 minutes. The reaction mixture was concentrated under reduced pressure, diluted with ethyl acetate (10 mL) and water (5 mL). After stirring for 30 min, the organic layer was separated, washed with a saturated aqueous brine solution (10 mL), dried over Na₂SO₄, and concentrated under reduced pressure to afford the title compound as a solid (1.28 g, 5.29 mmol, 90%).

UPLC-MS (basic method, 2 min, ELSD): Rt: 0.98 min, m/z=242.2 [M+H]⁺.

¹H NMR (400 MHz, Chloroform-d) δ 3.63 (dt, J=9.2, 5.1 Hz, 1H), 3.58 (s, 2H), 3.55 (s, 2H), 1.90-1.85 (m, 2H), 1.83-1.75 (m, 2H), 1.52-1.44 (m, 2H), 1.42 (s, 9H), 1.38-1.23 (m, 2H).

### N-(5-Cyclopropyl-1H-pyrazol-3-yl)-2-methylsulfonyl-pyrimidin-4-amine

To a solution of 4-chloro-2-(methylsulfonyl)pyrimidine (400 mg, 2.08 mmol) in DMSO (4 mL) was added 3-amino-5-cyclopropyl-1H-pyrazole (256 mg, 2.08 mmol) and N,N-diisopropylethylamine (0.54 mL, 3.1 mmol), The reaction mixture was heated to 60° C. and stirred for 2 hours. The reaction mixture was cooled to room temperature and poured into ice water resulting in precipitation. The precipitate was isolated by filtration and the filter cake was washed with CH₂Cl₂ (10 mL). The material was purified by trituration in hexanes to afford the title compound as a solid (210 mg, 0.729 mmol, 35%).

UPLC-MS (basic method, 2 min): Rt: 0.77 min, m/z=280.1 [M+H]⁺.

¹H NMR (400 MHz, DMSO-d₆) δ 12.22 (s, 1H), 10.56 (s, 1H), 8.40 (s, 1H), 7.56 (d, J=5.9 Hz, 1H), 5.75 (s, 1H), 3.31 (s, 3H), 1.96-1.83 (m, 1H), 0.97-0.86 (m, 2H), 0.75-0.60 (m, 2H).

### tert-Butyl 7-[4-[(5-cyclopropyl-1H-pyrazol-3-yl)amino]pyrimidin-2-yl]oxy-2-azaspiro[3.5]nonane-2-carboxylate

To a stirred solution of N-(5-cyclopropyl-1H-pyrazol-3-yl)-2-methylsulfonyl-pyrimidin-4-amine (170 mg, 0.609 mmol) and tert-butyl 7-hydroxy-2-azaspiro[3.5]nonane-2-carboxylate (441 mg, 1.83 mmol) in DMF (1 mL) was added a 60% suspension of sodium hydride in mineral oil (73 mg, 1.8 mmol) under a nitrogen atmosphere. The reaction mixture was stirred overnight at room temperature. The reaction was then quenched with water (5 mL) and extracted with CH₂Cl₂ (2×20 mL). The combined organic extracts were washed with a saturated aqueous brine solution (2×10 mL), dried over Na₂SO₄ and concentrated to dryness under reduced pressure. The residue was purified by normal-phase column chromatography, over silica gel, eluting with 98:2 CH₂Cl₂:MeOH. The residue was further purified by trituration in dichloromethane to afford the title compound as a solid (95 mg, 0.22 mmol, 35%).

UPLC-MS (basic method, 2 min): Rt: 1.09 min, m/z=441.4 [M+H]⁺.

¹H NMR (400 MHz, Chloroform-d) δ 8.32-7.64 (m, 1H), 6.42 (s, 1H), 5.94 (s, 1H), 4.88 (s, 1H), 3.57 (dd, J=9.3, 6.4 Hz, 4H), 2.12-1.78 (m, 4H), 1.79 (d, J=5.7 Hz, 1H), 1.74-1.46 (m, 4H), 1.41-1.32 (m, 9H), 0.96-0.86 (m, 2H), 0.70-0.60 (m, 2H).

### N-(5-Cyclopropyl-1H-pyrazol-3-yl)-2-[(2-methyl-2-azaspiro[3.5]nonan-7-yl)oxy]pyrimidin-4-amine

To a stirred solution of tert-butyl 7-[4-[(5-cyclopropyl-1H-pyrazol-3-yl)amino]pyrimidin-2-yl]oxy-2-azaspiro[3.5]nonane-2-carboxylate (75 mg, 0.17 mmol) in THF (1 mL) was added a 2.4 M solution of lithium aluminum hydride in THF (0.80 mL, 1.7 mmol) at 0° C. The reaction was heated to reflux and stirred for 4 hours. Afterwards, reaction was cooled to 0° C. and quenched with water (0.15 mL) followed by a 5% w/v NaOH solution (0.5 mL). After stirring for 30 minutes, MgSO₄ (0.5 g) was added to it and the reaction mixture was left for stirring for 16 hours. The reaction mixture was filtered through celite, and the filter cake was washed with CH₂Cl₂ (20 mL) followed by MeOH (10 mL). The filtrate was concentrated to dryness under reduced pressure. The residue was loaded onto an SCX-2 cartridge. The cartridge was washed with MeOH (20 mL) then the compound was eluted with 2 M NH₃ in MeOH. The eluent was concentrated to dryness under reduced pressure The residue was triturated in 1:1 CH₂Cl₂:iso-hexane to afford the title compound as a solid (20 mg, 54 μmol, 31%).

UPLC-MS (basic method, 4 min): Rt: 1.21 min, m/z=355.3 [M+H]⁺.

¹H NMR (400 MHz, Chloroform-d) δ 7.96 (dd, J=5.9, 0.8 Hz, 1H), 6.44 (s, 1H), 5.93 (s, 1H), 4.85 (s, 1H), 3.04 (d, J=14.7 Hz, 4H), 2.32 (s, 3H), 2.03-1.86 (m, 4H), 1.85-1.75 (m, 1H), 1.63-1.48 (m, 4H), 1.11-0.84 (m, 2H), 0.78-0.57 (m, 2H).

### Example 22: Synthesis of Compound 44

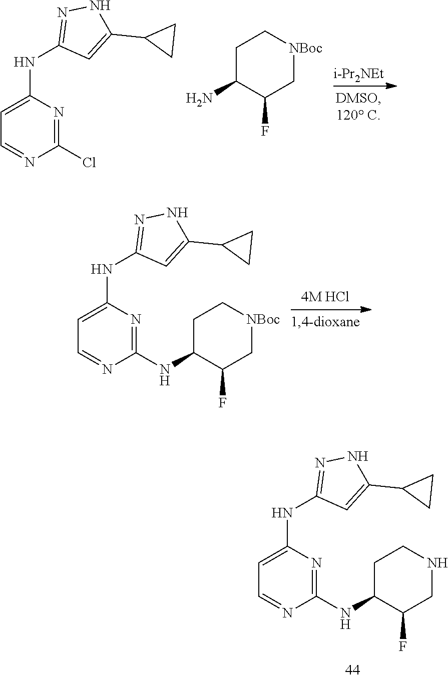 [SMILES: "i-Pr2NEt" ; Clc1nccc(Nc2cc(C3CC3)[nH]n2)n1 ; CC(C)(C)OC(=O)N1CC[C@H](N)[C@H](F)C1 ; CC(C)(C)OC(=O)N1CC[C@H](Nc2nccc(Nc3cc(C4CC4)[nH]n3)n2)[C@H](F)C1 ; F[C@@H]1CNCC[C@@H]1Nc1nccc(Nc2cc(C3CC3)[nH]n2)n1]

### 2-[[3-[4-[(5-Cyclopentyl-1H-pyrazol-3-yl)amino]pyrimidin-2-yl]-3-azabicyclo[3.1.1]heptan-1-yl]methyl]isoindoline-1,3-dione

To a solution of 2-chloro-N-(5-cyclopropyl-1H-pyrazol-3-yl)pyrimidin-4-amine (1.55 g, 6.59 mmol) and tert-butyl (3R,4S)-4-amino-3-fluoropiperidine-1-carboxylate (1.73 g, 7.91 mmol) in DMSO (65 mL) was added N,N-diisopropylethylamine (3.4 mL, 20 mmol). The resulting solution was heated to 120° C. and stirred for 6 days. The reaction mixture was poured into ice water (400 mL), and the solid that precipitated was filtered and washed with water (200 mL) and dried under reduced pressure. The precipitate was purified by basic reversed-phase prepHPLC to afford the title compound as a solid (79 mg, 0.19 mmol, 2.8%).

¹H NMR (400 MHz, DMSO-d₆) δ 11.90 (s, 1H), 9.31 (s, 1H), 7.79 (d, J=5.7 Hz, 1H), 6.60-5.91 (m, 2H), 4.83 (m, 1H), 4.23 (s, 1H), 4.04 (s, 2H), 3.17 (d, J=5.2 Hz, 1H), 3.13-2.73 (m, 2H), 1.94-1.60 (m, 3H), 1.40 (s, 9H), 0.99-0.54 (m, 4H).

¹⁹F NMR (376 MHz, DMSO-d₆) δ −202.0-−204.2 (m).

UPLC-MS (basic method, 4 min): rt=1.65 min, m z: 418.3 [M+H]⁺.

### N⁴-(5-Cyclopropyl-1H-pyrazol-3-yl)-N²-[(3R,4S)-3-fluoro-4-piperidyl]pyrimidine-2,4-diamine

To tert-butyl (3R,4S)-4-[[4-[(5-cyclopropyl-1H-pyrazol-3-yl)amino]pyrimidin-2-yl]amino]-3-fluoro-piperidine-1-carboxylate (34 mg, 82 μmol) was added 4M HCl in 1,4-Dioxane (0.20 mL, 0.81 mmol). The resulting mixture was stirred at room temperature for 2 hours. The volatiles were removed under reduced pressure to afford an oil, which was dissolved in methanol (0.5 mL) and loaded onto an SCX-2 cartridge. The cartridge was washed with MeOH (10 mL) and the compound was eluted with 2M NH₃ in MeOH (10 mL). The volatiles were removed under reduced pressure to afford the title compound as a powder (21 mg, 64 μmol, 78%).

¹H NMR (400 MHz, DMSO-d₆) δ 11.87 (s, 1H), 9.30 (s, 1H), 7.76 (s, 1H), 6.41 (s, 1H), 6.11 (s, 2H), 4.67 (d, J=50.5 Hz, 1H), 3.94 (d, J=31.1 Hz, 1H), 3.12 (t, J=12.6 Hz, 1H), 2.94 (d, J=12.9 Hz, 1H), 2.78-2.58 (m, 1H), 2.53 (t, J=13.7 Hz, 1H), 1.80 (s, 1H), 1.58 (s, 2H), 0.86 (s, 2H), 0.63 (s, 2H).

UPLC-MS (basic method, 4 min): rt=1.02 min, m z: 318.2 [M+H]⁺.

### Example 23: Synthesis of Compound 49

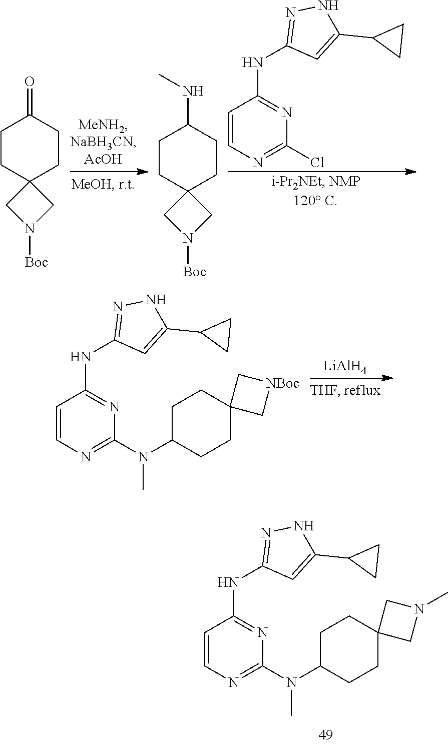

### tert-Butyl 7-(Methylamino)-2-azaspiro[3.5]nonane-2-carboxylate

To a stirred solution of tert-butyl 7-oxo-2-azaspiro[3.5]nonane-2-carboxylate (500 mg, 2.09 mmol) in methanol (4 mL) was added acetic acid (0.1 mL) and 2.0 M methylamine in methanol (1.6 mL). The reaction mixture was stirred at room temperature for 2 hours under nitrogen atmosphere, after which, sodium cyanoborohydride (263 mg, 4.18 mmol) was added portion wise. The reaction mixture was stirred for 16 hours at room temperature. The reaction mixture was quenched with a saturated aqueous NaHCO₃ solution, then concentrated to dryness under reduced pressure. The residue was suspended in water (5 mL), extracted with ethyl acetate (20 mL). The organic layer was dried over Na₂SO₄ and concentrated to dryness under reduced pressure to afford the title compound as an oil (500 mg, 1.97 mmol, 94%).

¹H NMR (400 MHz, Chloroform-d) δ 4.07-3.07 (m, 5H), 2.44 (s, 3H), 2.07-1.77 (m, 4H), 1.52-1.42 (m, 2H), 1.41 (d, J=0.7 Hz, 9H), 1.25-1.12 (m, 2H).

UPLC-MS (basic method, 2 min, ELSD): Rt: 0.84 min, m/z=255.5 [M+H]⁺.

### tert-Butyl 7-[[4-[(5-Cyclopropyl-1H-pyrazol-3-yl)amino]pyrimidin-2-yl]-methyl-amino]-2-azaspiro[3.5]nonane-2-carboxylate

To a stirred solution of tert-butyl 7-(methylamino)-2-azaspiro[3.5]nonane-2-carboxylate (463 mg, 1.82 mmol) and 2-chloro-N-(5-cyclopropyl-1H-pyrazol-3-yl)pyrimidin-4-amine (330 mg, 1.40 mmol) in NMP (3 mL) was added N,N-diisopropylethylamine (0.73 mL, 4.2 mmol). The reaction mixture was heated to 120° C. and stirred for 48 hours. The reaction was cooled down to room temperature and poured onto ice water. The precipitate was collected by filtration, then washed with water (10 mL) and iso-hexane (20 mL). The crude residue was purified by normal-phase column chromatography, over silica gel, eluting 19:1 CH₂Cl₂:2 M NH₃ in MeOH to afford the title compound as a solid (170 mg, 0.375 mmol, 27%).

¹H NMR (400 MHz, Chloroform-d) δ 7.87 (d, J=5.7 Hz, 1H), 6.18-5.89 (m, 2H), 4.46 (s, 1H), 3.63 (s, 2H), 3.54 (s, 2H), 2.90 (s, 3H), 2.03-1.91 (m, 2H), 1.86-1.74 (m, 1H), 1.71-1.55 (m, 4H), 1.52-1.40 (m, 2H), 1.39 (s, 9H), 0.99-0.84 (m, 2H), 0.77-0.62 (m, 2H).

UPLC-MS (basic method, 2 min): Rt: 1.14 min, m/z=454.4 [M+H]⁺.

### N₄-(5-Cyclopropyl-1H-pyrazol-3-yl)-N₂-methyl-N₂-(2-methyl-2-azaspiro[3.5]nonan-7-yl)pyrimidine-2,4-diamine

To a solution of tert-butyl 7-[[4-[(5-cyclopropyl-1H-pyrazol-3-yl)amino]pyrimidin-2-yl]-methyl-amino]-2-azaspiro[3.5]nonane-2-carboxylate (150 mg, 0.331 mmol) in THF (6 mL) was added lithium aluminum hydride (126 mg, 3.31 mmol) at 0° C. The reaction mixture was then heated to reflux and stirred for 3 hours. The reaction was then cooled to 0° C., then quenched by dropwise addition of water (140 μL), followed by a 15% w/v aqueous solution of NaOH (140 μL), followed by water (420 μL). The reaction mixture was warmed to room temperature and stirred for 15 minutes. MgSO₄ (ca. 1 g) was then added, and the reaction mixture was stirred for 16 hours. The reaction mixture was filtered through a pad of celite, then the filter cake was washed with DCM (20 mL), followed by EtOH (20 mL), then the filtrate was concentrated to dryness under reduced pressure. The crude residue was dissolved in MeOH (2 mL), then loaded onto an SCX-2 cartridge. The cartridge was washed with MeOH (20 mL), then the compound was eluted with a 2M solution of NH₃ in MeOH (20 mL). The eluent was concentrated to dryness under reduced pressure. The residue was purified by trituration with iso-hexane:CH₂Cl₂ (1:1) to afford the title compound as a solid (71 mg, 0.19 mmol, 57%).

¹H NMR (400 MHz, Methanol-d₄) δ 7.82 (d, J=5.8 Hz, 1H), 6.18 (s, 1H), 6.07 (d, J=5.7 Hz, 1H), 4.51 (s, 1H), 3.23 (s, 2H), 3.09 (d, J=1.1 Hz, 2H), 2.92 (s, 3H), 2.40 (d, J=0.8 Hz, 3H), 2.06 (d, J=9.5 Hz, 2H), 1.98-1.80 (m, 1H), 1.76-1.50 (m, 6H), 1.06-0.84 (m, 2H), 0.81-0.64 (m, 2H).

UPLC-MS (basic method, 4 min): Rt: 1.21 min, m/z=368.4 [M+H]⁺.

### Example 24: Synthesis of Compound 65

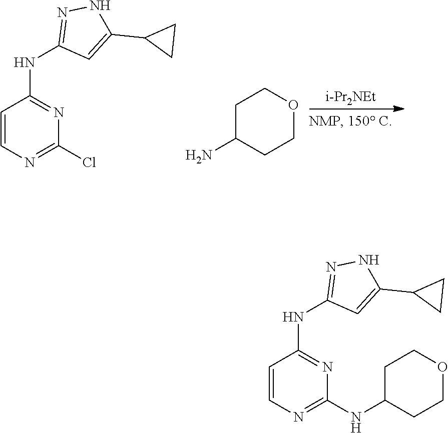 [SMILES: "i-Pr2NEt" ; Clc1nccc(Nc2cc(C3CC3)[nH]n2)n1 ; NC1CCOCC1 ; c1cc(Nc2cc(C3CC3)[nH]n2)nc(NC2CCOCC2)n1]

### N₄-(5-Cyclopropyl-1H-pyrazol-3-yl)-N₂-tetrahydropyran-4-yl-pyrimidine-2,4-diamine

To 2-chloro-N-(5-cyclopropyl-1H-pyrazol-3-yl)pyrimidin-4-amine (185 mg, 0.785 mmol) in NMP (3 mL) was added in N,N-diisopropylethylamine (0.45 mL, 2.6 mmol) and 4-aminotetrahydropyran (87 mg, 1.1 mmol) and the reaction was heated to 150° C. and stirred for 72 hours. The reaction mixture was poured into cold water (45 mL) then filtered. The filtrate was extracted with EtOAc (2×20 mL) and the solid was dissolved in EtOAc (20 mL). The organics were combined, dried over Na₂SO₄ and evaporated to dryness under reduced pressure. The residue was purified by normal phase automated flash column chromatography on silica, eluting with MeOH (0:100 to 1:4) in CH₂Cl₂ to give the title compound as a solid (16 mg, 42 μmol, 81%).

¹H NMR (400 MHz, DMSO-d₆) δ 11.90 (s, 1H), 9.29 (s, 1H), 7.78 (s, 1H), 6.57 (s, 1H), 6.13 (s, 1H), 5.75 (s, 1H), 3.91-3.84 (m, 3H), 3.42-3.32 (m, 1H), 1.88-1.80 (m, 4H), 1.52-1.42 (m, 2H), 0.91 (s, 2H), 0.68-0.63 (m, 2H).

UPLC-MS (basic method, 4 min): Rt: 1.20, m/z=301.2 [M+H]⁺.

### Example 25: Degradation of MycC/MycN Protein

For L363 cells (suspension), 1E6 cells were plated into each well of a 6 well plate. For SK—N-BE(2) cells (adherent), 5E5 cells were plated into each well of a 6 well dish. Cells were cultured for 24 hrs then treated with exemplary compounds at 0.1, 0.5, 1.0, 3.0 and 6.0 mM final assay concentrations plus DMSO control. All compounds were diluted to 10 mM in DMSO. Cells were treated with the compound for 24 hrs, then for both cell types, media containing cells were removed from wells into a 15 ml centrifuge tube, and remaining adherent SK—N-BE(2) cells were also scraped from the wells into the appropriate tubes. Tubes were centrifuged, cells were washed with PBS then re-centrifuged to pellet cells. A RIPA buffer cocktail was added to the cells on ice for 5 mins. Cell lysates were clarified by centrifugation and stored at −80 degrees until required. BCA assay was performed on each lysate to determine the protein concentration.

For validation studies of antibodies, lysates and antibodies were used at a number of concentrations (Lysates: 2, 1, 0.2 mg/ml; Antibodies: 1 in 50, 1 in 200 dilution). Once an appropriate lysate concentration and antibody concentration was determined, samples were screened in the JESS technology (Protein Simple, San Jose, CA—https://www.proteinsimple.com/jess.html).

Target protein antibodies (n-myc and c-myc) were detected in the chemiluminescence channel and loading controls (tubulin and GAPDH) were detected using a Near Infra Red (NIR) labeled secondary antibody.

### Cell Proliferation/Viability Measures

For SK—N-EB2 adherent cells, 5E5 cells in 1.9 mls media were plated into 6 well dishes and incubated for 24 hrs. Exemplary compounds were added (100 ul, 1 in 20 dilution in media) to each well (final assay concentration 6, 3, 1 and 0.5 mM) together with DMSO control wells and incubated for 24 hrs. Cells were scrapped off the plate, centrifuged, washed with PBS, centrifuged, then a RIPA buffer cocktail added to the cells (100 ul), centrifuged and stored at −80 for later analysis. For L363 suspension cells, 1E6 cells were plated into 24 well dishes (950 ul media) and incubated for 24 hrs. Exemplary compounds were added (50 ul, 1 in 20 dilution in media) to each well (final assay concentration 6, 3, 1 and 0.5 mM) together with DMSO control wells and incubated for 24 hrs. Cells were aspirated into tubes, centrifuged, washed with PBS, centrifuged, then a RIPA buffer cocktail added to the cells (100 ul), centrifuged and stored at −80 for later analysis.

For Western blot, a BCA (total protein) assay was run on all samples, these were then run in batches of 5 compounds plus control (DMSO) on each Western blot run—n-myc and c-myc were run in separate experiments in the chemiluminescence channel. Both tubulin and GAPDH were run as loading controls in all lanes in the near infra red channel.

For cytotox, both SK—N-EB2 or L363 cells were run in 384 well format. For SK—N-EB2 cells, 5000 cells per well (in 30 ul media) were incubated for 24 hrs prior to the addition of compounds (10 ul, 1 in 4 dilution, 10 mM top final concentration, 1 in 2 dilutions) for 24 hrs. For L363 cells, 2000 cells per well (30 ul media) were incubated for 24 hrs prior to the addition of compounds (10 ul, 10 mM 1 in 2 dilutions) for 24 hrs. In both cases Promega Cell Titer GLO was added according to the manufacturer's instructions and the plates read immediately in the luminometer.

TABLE 1
Percent Degradation of MycC/MycN Protein by Various Compounds

| | % | % | % | % | % | % | % | % |
|---|---|---|---|---|---|---|---|---|
| | Degradation | Degradation | Degradation | Degradation | Degradation | Degradation | Degradation | Degradation |
| | of MycN | of MycC | of MycN | of MycC | of MycN | of MycC | of MycN | of MycC |
| Cmpd | (SKNBE2) | (L-363) | (SKNBE2) | (L-363) | (SKNBE2) | (L-363) | (SKNBE2) | (L-363) |
| No. | 24 h, 6 μM | 24 h, 6 μM | 24 h, 3 μM | 24 h, 3 μM | 24 h, 1 μM | 24 h, 1 μM | 24 h, 0.5 μM | 24 h, 0.5 μM |
| 1 | **** | * | ** | * | * | ND | ** | ND |
| 2 | *** | ND | ** | ND | * | ND | ** | ND |
| 3 | ** | ND | * | ND | * | ND | * | ND |
| 4 | ** | * | * | ** | ND | * | ND | ND |
| 20 | **** | **** | **** | **** | **** | **** | **** | **** |
| 28 | * | ** | ND | * | ND | * | ND | * |
| 29 | **** | **** | **** | **** | * | **** | ND | *** |
| 30 | *** | **** | ND | **** | * | **** | * | **** |
| 31 | **** | **** | **** | **** | ND | *** | * | *** |
| 32 | **** | **** | **** | **** | *** | *** | ND | *** |
| 33 | **** | **** | **** | ** | ** | * | ** | ND |
| 34 | **** | **** | **** | **** | **** | *** | **** | * |
| 35 | ** | * | ** | * | * | ND | ** | ND |
| 36 | **** | *** | ** | ** | * | *** | * | ** |
| 37 | ** | * | ** | ** | * | ND | ND | No Data |
| 38 | **** | *** | **** | ** | *** | *** | ** | ** |
| 39 | **** | **** | **** | ** | * | ** | ** | ** |
| 40 | **** | **** | **** | **** | **** | * | *** | ND |
| 41 | **** | **** | ** | ND | * | ND | * | ND |
| 42 | *** | ND | ** | ND | ** | ND | ND | ND |
| 43 | **** | ** | *** | ** | ** | ** | * | * |
| 44 | *** | * | ** | ** | ** | ** | * | * |
| 49 | **** | **** | **** | **** | **** | **** | *** | *** |
| 65 | **** | **** | **** | **** | *** | **** | ** | ** |

Key: ****: degradation 80-100%
***: degradation 50-79%
**: degradation 20-49%
*: degradation <20%
ND—No Degradation

It will be appreciated that compounds reported as a salt form (e.g., a TFA salt) may or may not have a 1:1 stoichiometry, and/or for example, reported potency concentrations or other assay results may be, e.g., slightly higher or lower.

The practice of the present disclosure will employ, unless otherwise indicated, conventional methods of organic chemistry, protein chemistry, biochemistry, recombinant DNA techniques and pharmacology, within the skill of the art. While the disclosure has been particularly shown and described with reference to a preferred embodiment and various alternate embodiments, it will be understood by persons skilled in the relevant art that various changes in form and details can be made therein without departing from the spirit and scope of the disclosure.

All references, issued patents and patent applications cited within the body of the instant specification are hereby incorporated by reference in their entirety, for all purposes.

What is claimed is:

1. A compound of Formula I: 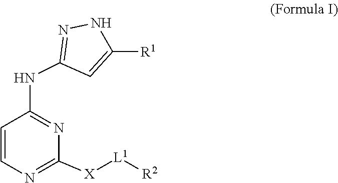
or a pharmaceutically acceptable salt, stereoisomer, and/or N-oxide thereof, wherein:
L¹ is a bond or C₁-C₆ alkylene;
X is NR^{A} or O;
R¹ is C₃-C₆ cycloalkyl;
R² is 5-10 membered monocyclic, bicyclic or spirocyclic heterocyclyl having at least one nitrogen, wherein the heterocyclyl is optionally substituted by one or two halo, oxo, hydroxyl, or C₁-C₄ alkyl; and
R^{A} is H and C₁-C₆ alkyl;
wherein when R² is a 6-membered monocyclic heterocyclyl, R¹ is cyclopentyl.

2. The compound of claim 1, wherein R¹ is cyclopropyl.

3. The compound of claim 1, wherein R¹ is cyclopenyl.

4. The compound of any one of claims 1-3, wherein R² is 5-6 membered heterocyclyl having at least one nitrogen.

5. The compound of any one of claims 1-3, wherein R² is 6-10 membered spiroheterocycle having at least one nitrogen, 6-10 membered fused bicyclic heterocycle having at least one nitrogen, or 6-10 membered bridged heterocycle having at least one nitrogen, each of which is optionally substituted by one or two halo, oxo, hydroxyl, or C₁-C₄ alkyl.

6. The compound of any one of claims 1-3, wherein R² is 6-10 membered bridged heterocycle having at least one nitrogen, optionally substituted by one or two halo, oxo, hydroxyl, or C₁-C₄ alkyl.

7. The compound of any one of claims 1-6, wherein R^{A} is H.

8. The compound of any one of claims 1-6, wherein R^{A} is C₁-C₆ alkyl.

9. The compound of claim 8, wherein R^{A} is methyl.

10. The compound of claim 1, selected from the group consisting of: 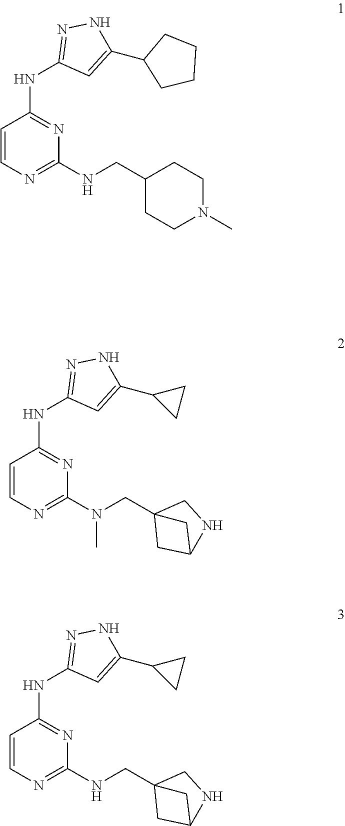 [SMILES: CN1CCC(CNc2nccc(Nc3cc(C4CCCC4)[nH]n3)n2)CC1 ; CN(CC12CNC(C1)C2)c1nccc(Nc2cc(C3CC3)[nH]n2)n1 ; c1cc(Nc2cc(C3CC3)[nH]n2)nc(NCC23CNC(C2)C3)n1] 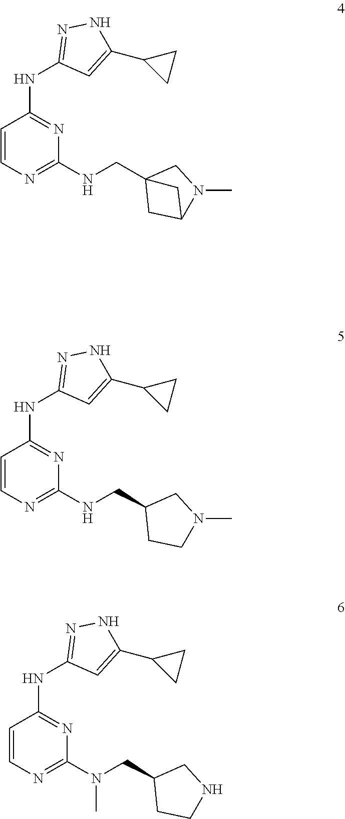 [SMILES: CN1CC2(CNc3nccc(Nc4cc(C5CC5)[nH]n4)n3)CC1C2 ; CN1CC[C@@H](CNc2nccc(Nc3cc(C4CC4)[nH]n3)n2)C1 ; CN(C[C@@H]1CCNC1)c1nccc(Nc2cc(C3CC3)[nH]n2)n1] 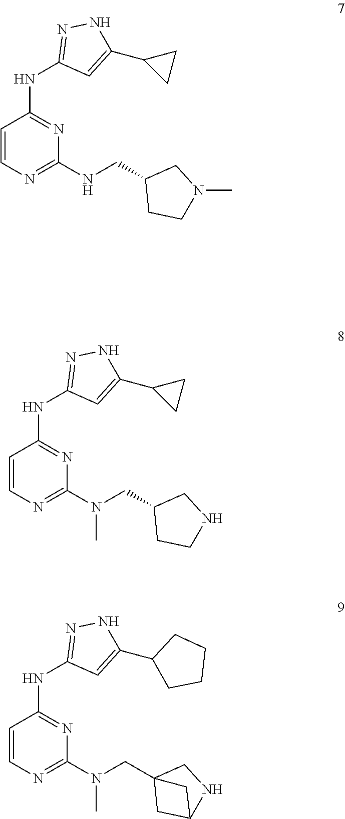 [SMILES: CN1CC[C@H](CNc2nccc(Nc3cc(C4CC4)[nH]n3)n2)C1 ; CN(C[C@H]1CCNC1)c1nccc(Nc2cc(C3CC3)[nH]n2)n1 ; CN(CC12CNC(C1)C2)c1nccc(Nc2cc(C3CCCC3)[nH]n2)n1] 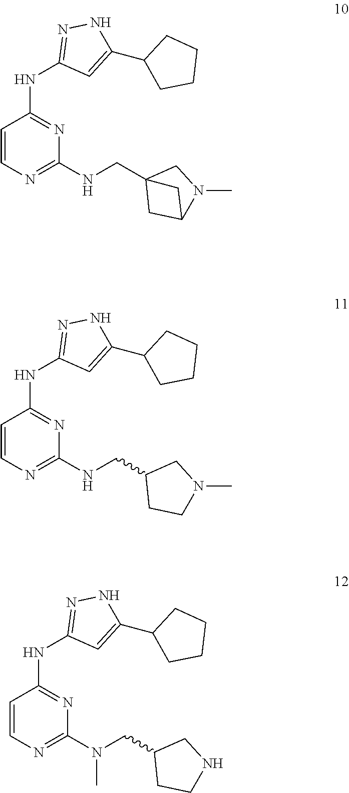 [SMILES: CN1CC2(CNc3nccc(Nc4cc(C5CCCC5)[nH]n4)n3)CC1C2 ; CN1CCC(CNc2nccc(Nc3cc(C4CCCC4)[nH]n3)n2)C1 ; CN(CC1CCNC1)c1nccc(Nc2cc(C3CCCC3)[nH]n2)n1] 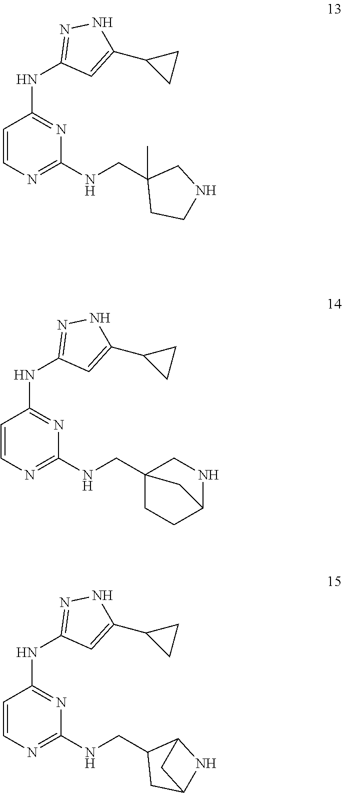 [SMILES: CC1(CNc2nccc(Nc3cc(C4CC4)[nH]n3)n2)CCNC1 ; c1cc(Nc2cc(C3CC3)[nH]n2)nc(NCC23CCC(C2)NC3)n1 ; c1cc(Nc2cc(C3CC3)[nH]n2)nc(NCC2CC3CC2N3)n1] 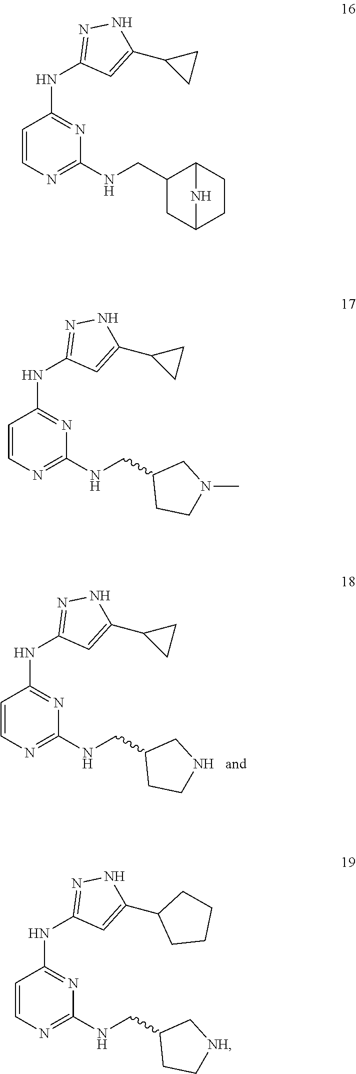 [SMILES: c1cc(Nc2cc(C3CC3)[nH]n2)nc(NCC2CC3CCC2N3)n1 ; CN1CCC(CNc2nccc(Nc3cc(C4CC4)[nH]n3)n2)C1 ; c1cc(Nc2cc(C3CC3)[nH]n2)nc(NCC2CCNC2)n1 ; c1cc(Nc2cc(C3CCCC3)[nH]n2)nc(NCC2CCNC2)n1]
or a pharmaceutically acceptable salt, stereoisomer, and/or N-oxide thereof.

11. A compound selected from the group consisting of 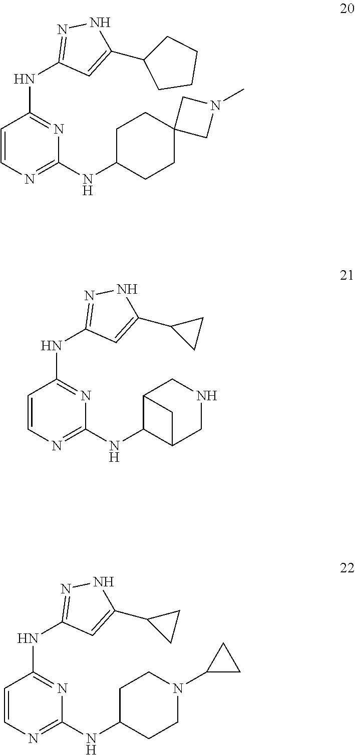 [SMILES: CN1CC2(CCC(Nc3nccc(Nc4cc(C5CCCC5)[nH]n4)n3)CC2)C1 ; c1cc(Nc2cc(C3CC3)[nH]n2)nc(NC2C3CNCC2C3)n1 ; c1cc(Nc2cc(C3CC3)[nH]n2)nc(NC2CCN(C3CC3)CC2)n1] 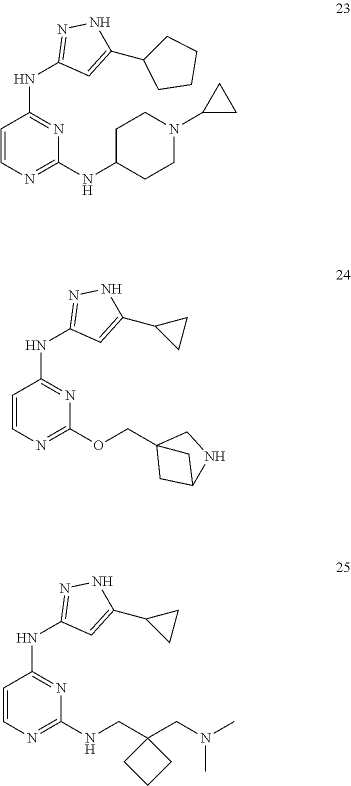 [SMILES: c1cc(Nc2cc(C3CCCC3)[nH]n2)nc(NC2CCN(C3CC3)CC2)n1 ; c1cc(Nc2cc(C3CC3)[nH]n2)nc(OCC23CNC(C2)C3)n1 ; CN(C)CC1(CNc2nccc(Nc3cc(C4CC4)[nH]n3)n2)CCC1] 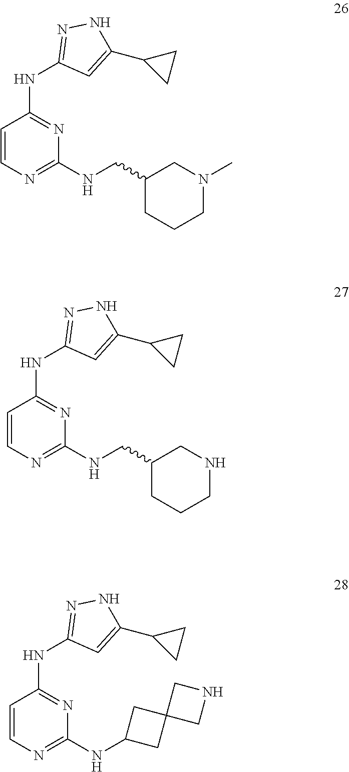 [SMILES: CN1CCCC(CNc2nccc(Nc3cc(C4CC4)[nH]n3)n2)C1 ; c1cc(Nc2cc(C3CC3)[nH]n2)nc(NCC2CCCNC2)n1 ; c1cc(Nc2cc(C3CC3)[nH]n2)nc(NC2CC3(CNC3)C2)n1] 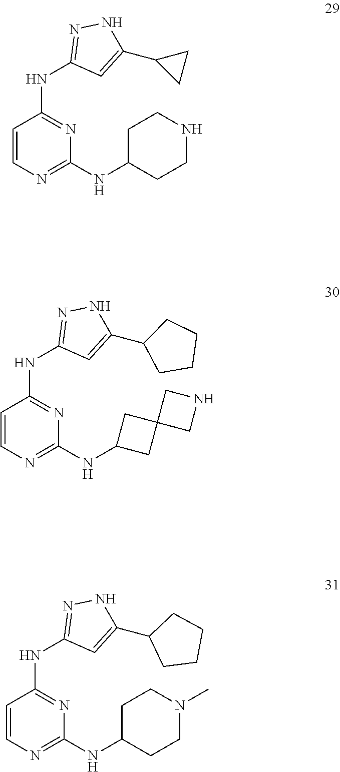 [SMILES: c1cc(Nc2cc(C3CC3)[nH]n2)nc(NC2CCNCC2)n1 ; c1cc(Nc2cc(C3CCCC3)[nH]n2)nc(NC2CC3(CNC3)C2)n1 ; CN1CCC(Nc2nccc(Nc3cc(C4CCCC4)[nH]n3)n2)CC1] 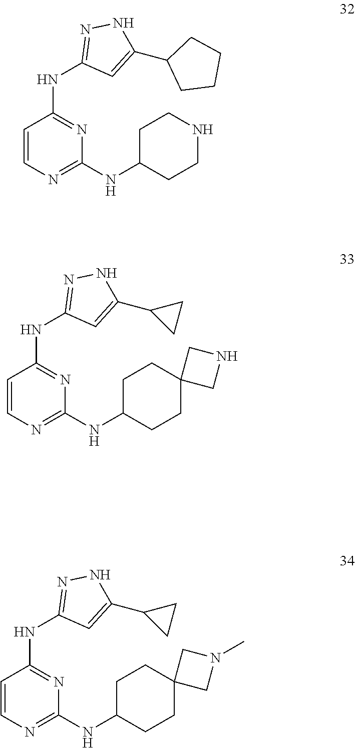 [SMILES: c1cc(Nc2cc(C3CCCC3)[nH]n2)nc(NC2CCNCC2)n1 ; c1cc(Nc2cc(C3CC3)[nH]n2)nc(NC2CCC3(CC2)CNC3)n1 ; CN1CC2(CCC(Nc3nccc(Nc4cc(C5CC5)[nH]n4)n3)CC2)C1] 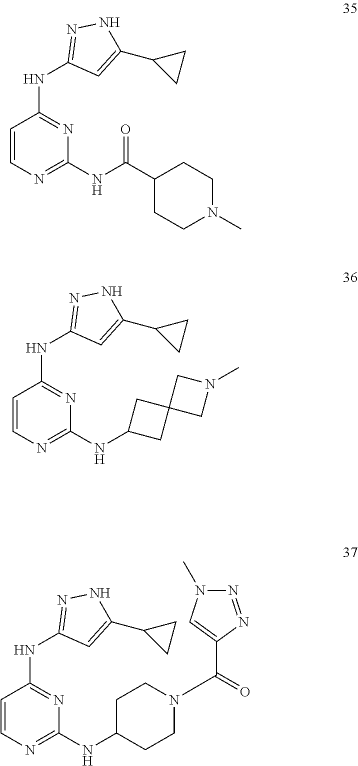 [SMILES: CN1CCC(C(=O)Nc2nccc(Nc3cc(C4CC4)[nH]n3)n2)CC1 ; CN1CC2(CC(Nc3nccc(Nc4cc(C5CC5)[nH]n4)n3)C2)C1 ; Cn1cc(C(=O)N2CCC(Nc3nccc(Nc4cc(C5CC5)[nH]n4)n3)CC2)nn1] 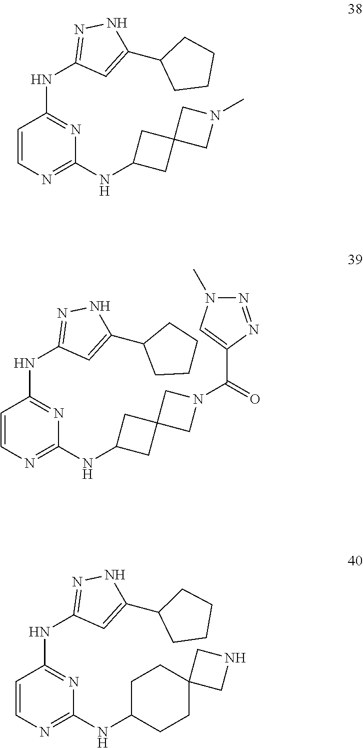 [SMILES: CN1CC2(CC(Nc3nccc(Nc4cc(C5CCCC5)[nH]n4)n3)C2)C1 ; Cn1cc(C(=O)N2CC3(CC(Nc4nccc(Nc5cc(C6CCCC6)[nH]n5)n4)C3)C2)nn1 ; c1cc(Nc2cc(C3CCCC3)[nH]n2)nc(NC2CCC3(CC2)CNC3)n1] 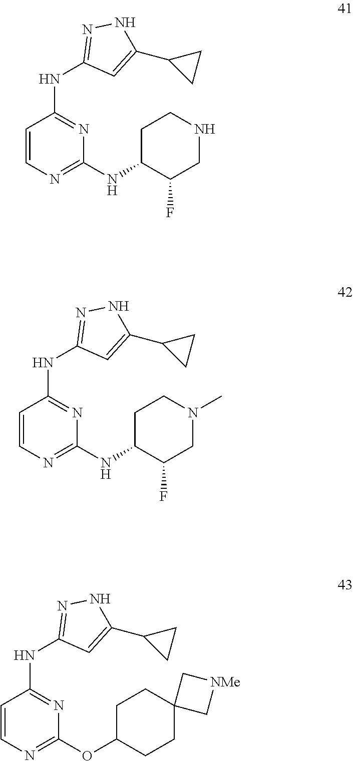 [SMILES: F[C@H]1CNCC[C@H]1Nc1nccc(Nc2cc(C3CC3)[nH]n2)n1 ; CN1CC[C@@H](Nc2nccc(Nc3cc(C4CC4)[nH]n3)n2)[C@@H](F)C1 ; CN1CC2(CCC(Oc3nccc(Nc4cc(C5CC5)[nH]n4)n3)CC2)C1] 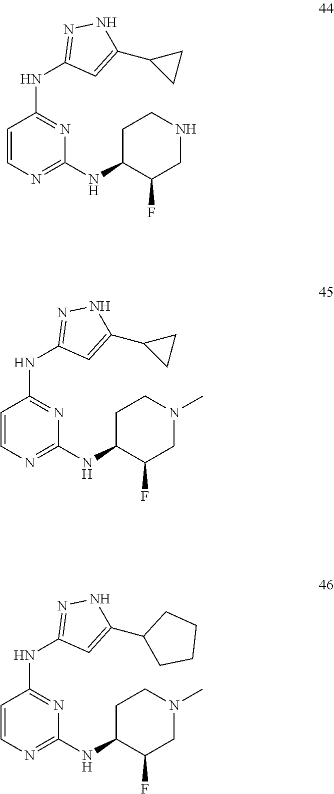 [SMILES: F[C@@H]1CNCC[C@@H]1Nc1nccc(Nc2cc(C3CC3)[nH]n2)n1 ; CN1CC[C@H](Nc2nccc(Nc3cc(C4CC4)[nH]n3)n2)[C@H](F)C1 ; CN1CC[C@H](Nc2nccc(Nc3cc(C4CCCC4)[nH]n3)n2)[C@H](F)C1] 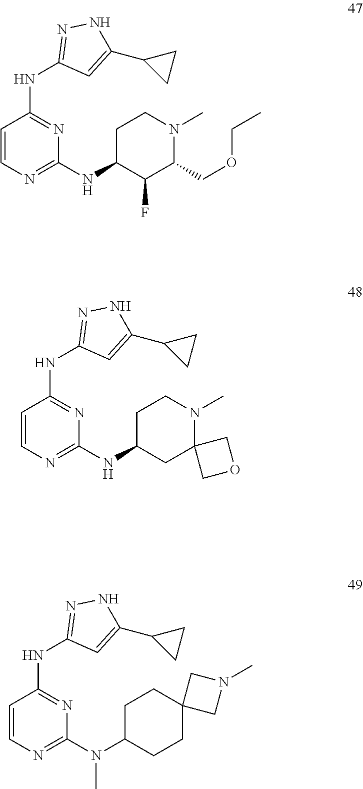 [SMILES: CCOC[C@@H]1[C@@H](F)[C@@H](Nc2nccc(Nc3cc(C4CC4)[nH]n3)n2)CCN1C ; CN1CC[C@H](Nc2nccc(Nc3cc(C4CC4)[nH]n3)n2)CC12COC2 ; CN1CC2(CCC(N(C)c3nccc(Nc4cc(C5CC5)[nH]n4)n3)CC2)C1] 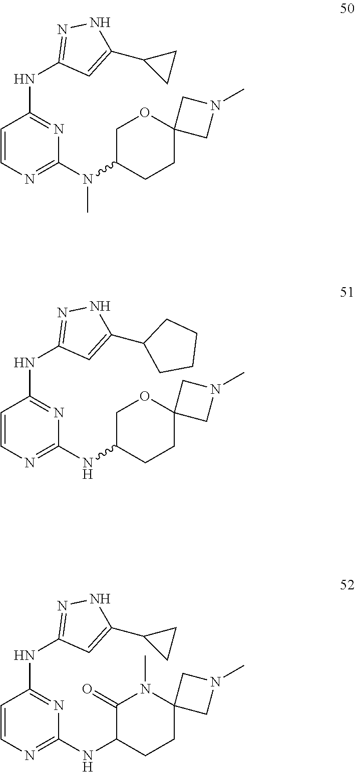 [SMILES: CN1CC2(CCC(N(C)c3nccc(Nc4cc(C5CC5)[nH]n4)n3)CO2)C1 ; CN1CC2(CCC(Nc3nccc(Nc4cc(C5CCCC5)[nH]n4)n3)CO2)C1 ; CN1CC2(CCC(Nc3nccc(Nc4cc(C5CC5)[nH]n4)n3)C(=O)N2C)C1] 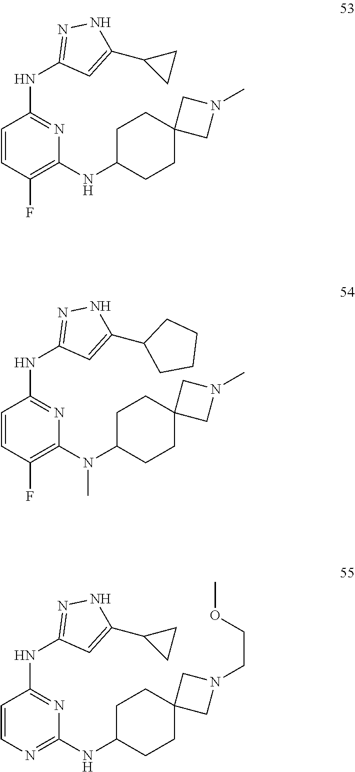 [SMILES: CN1CC2(CCC(Nc3nc(Nc4cc(C5CC5)[nH]n4)ccc3F)CC2)C1 ; CN1CC2(CCC(N(C)c3nc(Nc4cc(C5CCCC5)[nH]n4)ccc3F)CC2)C1 ; COCCN1CC2(CCC(Nc3nccc(Nc4cc(C5CC5)[nH]n4)n3)CC2)C1] 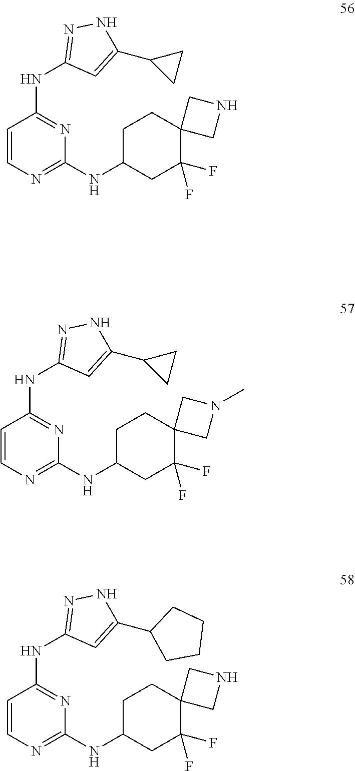 [SMILES: FC1(F)CC(Nc2nccc(Nc3cc(C4CC4)[nH]n3)n2)CCC12CNC2 ; CN1CC2(CCC(Nc3nccc(Nc4cc(C5CC5)[nH]n4)n3)CC2(F)F)C1 ; FC1(F)CC(Nc2nccc(Nc3cc(C4CCCC4)[nH]n3)n2)CCC12CNC2] 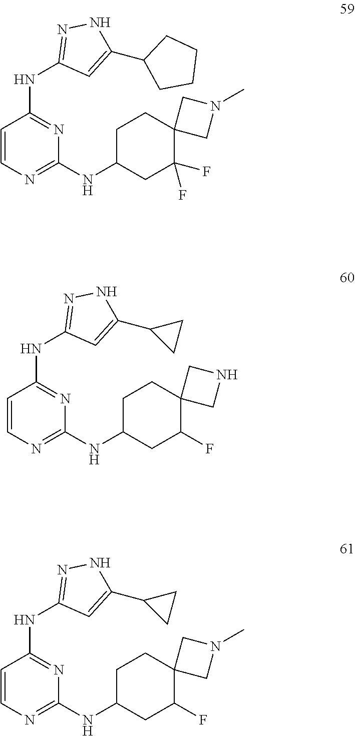 [SMILES: CN1CC2(CCC(Nc3nccc(Nc4cc(C5CCCC5)[nH]n4)n3)CC2(F)F)C1 ; FC1CC(Nc2nccc(Nc3cc(C4CC4)[nH]n3)n2)CCC12CNC2 ; CN1CC2(CCC(Nc3nccc(Nc4cc(C5CC5)[nH]n4)n3)CC2F)C1] 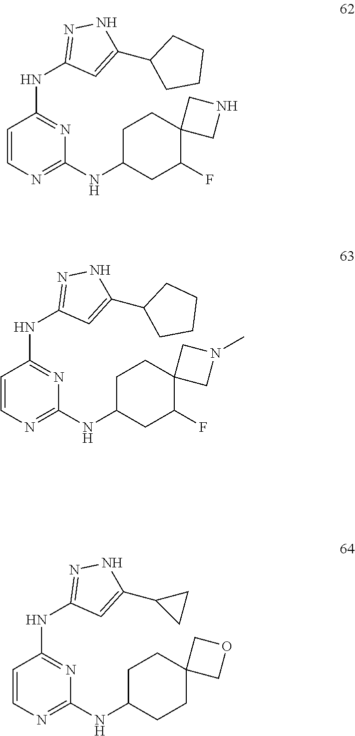 [SMILES: FC1CC(Nc2nccc(Nc3cc(C4CCCC4)[nH]n3)n2)CCC12CNC2 ; CN1CC2(CCC(Nc3nccc(Nc4cc(C5CCCC5)[nH]n4)n3)CC2F)C1 ; c1cc(Nc2cc(C3CC3)[nH]n2)nc(NC2CCC3(CC2)COC3)n1] 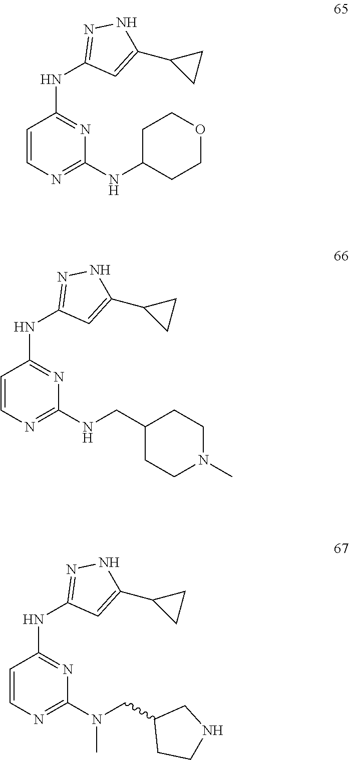 [SMILES: c1cc(Nc2cc(C3CC3)[nH]n2)nc(NC2CCOCC2)n1 ; CN1CCC(CNc2nccc(Nc3cc(C4CC4)[nH]n3)n2)CC1 ; CN(CC1CCNC1)c1nccc(Nc2cc(C3CC3)[nH]n2)n1] 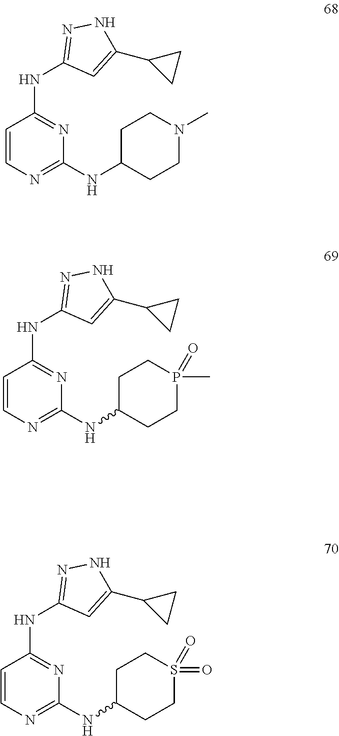 [SMILES: CN1CCC(Nc2nccc(Nc3cc(C4CC4)[nH]n3)n2)CC1 ; CP1(=O)CCC(Nc2nccc(Nc3cc(C4CC4)[nH]n3)n2)CC1 ; O=S1(=O)CCC(Nc2nccc(Nc3cc(C4CC4)[nH]n3)n2)CC1] 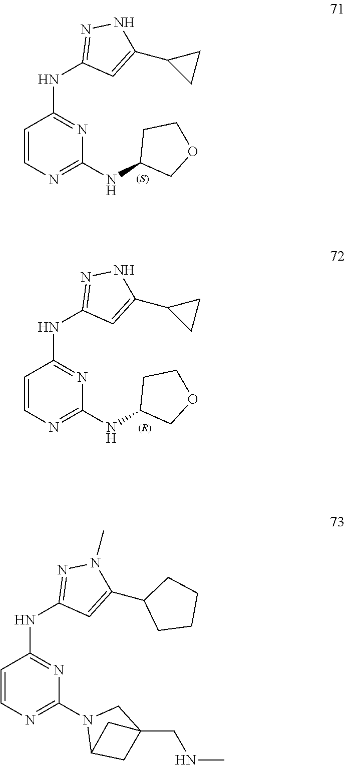
or a pharmaceutically acceptable salt, stereoisomer, and/or N-oxide thereof.

12. A pharmaceutical composition comprising a compound according to any one of claims 1-11, or a pharmaceutically acceptable salt, stereoisomer, and/or N-oxide thereof, and at least one pharmaceutically acceptable carrier or diluent.

13. The pharmaceutical composition of claim 12, wherein the composition is formulated for parenteral administration.

14. The pharmaceutical composition of claim 12, wherein the composition is formulated for intravenous administration.

15. The pharmaceutical composition of claim 12, wherein the composition is formulated for subcutaneous administration.

16. A method of treating a proliferative disease, comprising: administering to a subject with a proliferative disease a therapeutically effective amount of a compound according to any one of claims 1-11, or a pharmaceutically acceptable salt, stereoisomer and/or N-oxide thereof, or a therapeutically effective amount of the pharmaceutical composition of any one of claims 12-15.

17. The method of claim 16, wherein the proliferative disease is cancer.

18. The method of claim 17, wherein the cancer is selected from the group consisting of head and neck cancer, nervous system cancer, brain cancer, neuroblastoma, lung/mediastinum cancer, breast cancer, esophageal cancer, stomach cancer, liver cancer, biliary tract cancer, pancreatic cancer, small bowel cancer, large bowel cancer, colorectal cancer, gynecological cancer, genito-urinary cancer, ovarian cancer, thyroid gland cancer, adrenal gland cancer, skin cancer, melanoma, bone sarcoma, soft tissue sarcoma, pediatric malignancy, Hodgkin's disease, non-Hodgkin's lymphoma, myeloma, leukemia, and metastasis from an unknown primary site.

19. A method of modulating MycN in cells of a subject in need thereof, comprising: administering to a subject in need thereof an amount of a compound according to any one of claims 1-11, or a pharmaceutically acceptable salt, stereoisomer and/or N-oxide thereof, or a pharmaceutical composition according to any one of claims 12-15, that is effective to cause MycN modulation in cells of the subject.

20. The method of any one of claims 16-19, further comprising administering to the subject a second therapy.

21. The method of claim 20, wherein the second therapy is an antineoplastic therapy.

22. The method of claim 21, wherein the antineoplastic therapy is administration of one or more agents selected from a DNA topoisomerase I or II inhibitor, a DNA damaging agent, an immunotherapeutic agent, an antimetabolite or a thymidylate synthase (TS) inhibitor, a microtubule targeted agent, ionising radiation, an inhibitor of a mitosis regulator or a mitotic checkpoint regulator, an inhibitor of a DNA damage signal transducer, and an inhibitor of a DNA damage repair enzyme.

23. The method of claim 21, wherein the antineoplastic therapy is selected from the group consisting of immunotherapy, radiation therapy, photodynamic therapy, gene-directed enzyme prodrug therapy (GDEPT), antibody-directed enzyme prodrug therapy (ADEPT), gene therapy, and controlled diets.
